SEQUENCE LISTING (249 sequences)

SEQ ID NO: 1 - protein, 202 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTGHHHHHH

SEQ ID NO: 2 - protein, 248 aa, Mus musculus
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGS

SEQ ID NO: 3 - protein, 197 aa, Homo Sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 4 - protein, 306 aa, Homo Sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 193 aa, Mus musculus
RVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 6 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - protein, 449 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 8 - protein, 585 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 9 - protein, 459 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 10 - protein, 579 aa, Artificial Sequence
  Synthetic Polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 11 - protein, 786 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 12 - protein, 790 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSQLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGGGSGGGSGGGSGGGSGGGSGGGSMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 13 - protein, 10 aa, Artificial Sequence
  Synthetic Polypeptide
GLLSGRSDNH

SEQ ID NO: 14 - protein, 6 aa, Artificial Sequence
  Synthetic Polypeptide
LVPRGS

SEQ ID NO: 15 - protein, 469 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 16 - protein, 463 aa, Artificial Sequence
  Synthetic Polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 17 - protein, 451 aa, Artificial Sequence
  Synthetic Polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 18 - protein, 481 aa, Artificial Sequence
  Synthetic Polypeptide
CCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQALVPRGSLVPRGSLVPRGSGLLSGRSDNHGLLSGRSDNHGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 19 - protein, 639 aa, Homo Sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPENPPQPQVRFSVEQLGQDGRRRLTLKEQPTQLELPEGCQGLAPGTEVTYRLQLHMLSCPCKAKATRTLHLGKMPYLSGAAYNVAVISSNQFGPGLNQTWHIPADTHTEPVALNISVGTNGTTMYWPARAQSMTYCIEWQPVGQDGGLATCSLTAPQDPDPAGMATYSWSRESGAMGQEKCYYITIFASAHPEKLTLWSTVLSTYHFGGNASAAGTPHHVSVKNHSLDSVSVDWAPSLLSTCPGVLKEYVVRCRDEDSKQVSEHPVQPTETQVTLSGLRAGVAYTVQVRADTAWLRGVWSQPQRFSIEVQVSDWLIFFASLGSFLSILLVGVLGYLGLNRAARHLCPPLPTPCASSAIEFPGGKETWQWINPVDFQEEASLQEALVVEMSWDKGERTEPLEKTELPEGAPELALDTELSLEDGDRCKAKM

SEQ ID NO: 20 - protein, 719 aa, Mus musculus
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAKIKFLVEPLNQGGRRRLTMQGQSPQLAVPEGCRGRPGAQVKKHLVLVRMLSCRCQAQTSKTVPLGKKLNLSGATYDLNVLAKTRFGRSTIQKWHLPAQELTETRALNVSVGGNMTSMQWAAQAPGTTYCLEWQPWFQHRNHTHCTLIVPEEEDPAKMVTHSWSSKPTLEQEECYRITVFASKNPKNPMLWATVLSSYYFGGNASRAGTPRHVSVRNQTGDSVSVEWTASQLSTCPGVLTQYVVRCEAEDGAWESEWLVPPTKTQVTLDGLRSRVMYKVQVRADTARLPGAWSHPQRFSFEVQISRLSIIFASLGSFASVLLVGSLGYIGLNRAAWHLCPPLPTPCGSTAVEFPGSQGKQAWQWCNPEDFPEVLYPRDALVVEMPGDRGDGTESPQAAPECALDTRRPLETQRQRQVQALSEARRLGLAREDCPRGDLAHVTLPLLLGGVTQGASVLDDLWRTHKTAEPGPPTLGQEA

SEQ ID NO: 21 - protein, 839 aa, Homo Sapiens
KIDACKRGDVTVKPSHVILLGSTVNITCSLKPRQGCFHYSRRNKLILYKFDRRINFHHGHSLNSQVTGLPLGTTLFVCKLACINSDEIQICGAEIFVGVAPEQPQNLSCIQKGEQGTVACTWERGRDTHLYTEYTLQLSGPKNLTWQKQCKDIYCDYLDFGINLTPESPESNFTAKVTAVNSLGSSSSLPSTFTFLDIVRPLPPWDIRIKFQKASVSRCTLYWRDEGLVLLNRLRYRPSNSRLWNMVNVTKAKGRHDLLDLKPFTEYEFQISSKLHLYKGSWSDWSESLRAQTPEEEPTGMLDVWYMKRHIDYSRQQISLFWKNLSVSEARGKILHYQVTLQELTGGKAMTQNITGHTSWTTVIPRTGNWAVAVSAANSKGSSLPTRINIMNLCEAGLLAPRQVSANSEGMDNILVTWQPPRKDPSAVQEYVVEWRELHPGGDTQVPLNWLRSRPYNVSALISENIKSYICYEIRVYALSGDQGGCSSILGNSKHKAPLSGPHINAITEEKGSILISWNSIPVQEQMGCLLHYRIYWKERDSNSQPQLCEIPYRVSQNSHPINSLQPRVTYVLWMTALTAAGESSHGNEREFCLQGKANWMAFVAPSICIAIIMVGIFSTHYFQQKVFVLLAALRPQWCSREIPDPANSTCAKKYPIAEEKTQLPLDRLLIDWPTPEDPEPLVISEVLHQVTPVFRHPPCSNWPQREKGIQGHQASEKDMMHSASSPPPPRALQAESRQLVDLYKVLESRGSDPKPENPACPWTVLPAGDLPTHDGYLPSNIDDLPSHEAPLADSLEELEPQHISLSVFPSSSLHPLTFSCGDKLTLDQLKMRCDSLML

SEQ ID NO: 22 - protein, 851 aa, Mus musculus
NIDVCKLGTVTVQPAPVIPLGSAANISCSLNPKQGCSHYPSSNELILLKFVNDVLVENLHGKKVHDHTGHSSTFQVTNLSLGMTLFVCKLNCSNSQKKPPVPVCGVEISVGVAPEPPQNISCVQEGENGTVACSWNSGKVTYLKTNYTLQLSGPNNLTCQKQCFSDNRQNCNRLDLGINLSPDLAESRFIVRVTAINDLGNSSSLPHTFTFLDIVIPLPPWDIRINFLNASGSRGTLQWEDEGQVVLNQLRYQPLNSTSWNMVNATNAKGKYDLRDLRPFTEYEFQISSKLHLSGGSWSNWSESLRTRTPEEEPVGILDIWYMKQDIDYDRQQISLFWKSLNPSEARGKILHYQVTLQEVTKKTTLQNTTRHTSWTRVIPRTGAWTASVSAANSKGASAPTHINIVDLCGTGLLAPHQVSAKSENMDNILVTWQPPKKADSAVREYIVEWRALQPGSITKFPPHWLRIPPDNMSALISENIKPYICYEIRVHALSESQGGCSSIRGDSKHKAPVSGPHITAITEKKERLFISWTHIPFPEQRGCILHYRIYWKERDSTAQPELCEIQYRRSQNSHPISSLQPRVTYVLWMTAVTAAGESPQGNEREFCPQGKANWKAFVISSICIAIITVGTFSIRYFRQKAFTLLSTLKPQWYSRTIPDPANSTWVKKYPILEEKIQLPTDNLLMAWPTPEEPEPLIIHEVLYHMIPVVRQPYYFKRGQGFQGYSTSKQDAMYIANPQATGTLTAETRQLVNLYKVLESRDPDSKLANLTSPLTVTPVNYLPSHEGYLPSNIEDLSPHEADPTDSFDLEHQHISLSIFASSSLRPLIFGGERLTLDRLKMGYDSLMSNEA

SEQ ID NO: 23 - protein, 134 aa, Homo Sapiens
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 24 - protein, 148 aa, Mus musculus
PTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 25 - protein, 133 aa, Mus musculus
HGTVIESLESLNNYFNSSGIDVEEKSLFLDIWRNWQKDGDMKILQSQIISFYLRLFEVLKDNQAISNNISVIESHLITTFFSNSKAKKDAFMSIAKFEVNNPQVQRQAFNELIRVVHQLLPESSLRKRKRSRC

SEQ ID NO: 26 - protein, 143 aa, Homo Sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 27 - protein, 214 aa, Homo Sapiens
AVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDT

SEQ ID NO: 28 - protein, 214 aa, Mus musculus
AVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKE

SEQ ID NO: 29 - protein, 219 aa, Homo Sapiens
ELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQ

SEQ ID NO: 30 - protein, 215 aa, Mus musculus
ELCLYDPPEVPNATFKALSYKNGTILNCECKRGFRRLKELVYMRCLGNSWSSNCQCTSNSHDKSRKQVTAQLEHQKEQQTTTDMQKPTQSMHQENLTGHCREPPPWKHEDSKRIYHFVEGQSVHYECIPGYKALQRGPAISICKMKCGKTGWTQPQLTCVDEREHHRFLASEESQGSRNSSPESETSCPITTTDFPQPTETTAMTETFVLTMEYK

SEQ ID NO: 31 - protein, 240 aa, Homo Sapiens
LNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEA

SEQ ID NO: 32 - protein, 241 aa, Mus musculus
WSSKVLMSSANEDIKADLILTSTAPEHLSAPTLPLPEVQCFVFNIEYMNCTWNSSSEPQATNLTLHYRYKVSDNNTFQECSHYLFSKEITSGCQIQKEDIQLYQTFVVQLQDPQKPQRRAVQKLNLQNLVIPRAPENLTLSNLSESQLELRWKSRHIKERCLQYLVQYRSNRDRSWTELIVNHEPRFSLPSVDELKRYTFRVRSRYNPICGSSQQWSKWSQPVHWGSHTVEENPSLFALEA

SEQ ID NO: 33 - protein, 228 aa, Homo Sapiens [sequence omitted]

SEQ ID NO: 34 - protein, 229 aa, Mus musculus
ALTSTEDPEPPSVPVPTNVLIKSYNLNPVVCWEYQNMSQTPIFTVQVKVYSGSWTDSCTNISDHCCNIYEQIMYPDVSAWARVKAKVGQKESDYARSKEFLMCLKGKVGPPGLEIRRKKEEQLSVLVFHPEVVVNGESQGTMFGDGSTCYTFDYTVYVEHNRSGEILHTKHTVEKEECNETLCELNISVSTLDSRYCISVDGISSFWQVRTEKSKDVCIPPFHDDRKDS

SEQ ID NO: 35 - protein, 220 aa, Homo Sapiens
SQLPAPQHPKIRLYNAEQVLSWEPVALSNSTRPVVYQVQFKYTDSKWFTADIMSIGVNCTQITATECDFTAASPSAGFPMDFNVTLRLRAELGALHSAWVTMPWFQHYRNVTVGPPENIEVTPGEGSLIIRFSSPFDIADTSTAFFCYYVHYWEKGGIQQVKGPFRSNSISLDNLKPSRVYCLQVQAQLLWNKSNIFRVGHLSNISCYETMADASTELQQ

SEQ ID NO: 36 - protein, 313 aa, Mus musculus
ASSPDSFSQLAAPLNPRLHLYNDEQILTWEPSPSSNDPRPVVYQVEYSFIDGSWHRLLEPNCTDITETKCDLTGGGRLKLFPHPFTVFLRVRAKRGNLTSKWVGLEPFQHYENVTVGPPKNISVTPGKGSLVIHFSPPFDVFHGATFQYLVHYWEKSETQQEQVEGPFKSNSIVLGNLKPYRVYCLQTEAQLILKNKKIRPHGLLSNVSCHETTANASARLQQVILIPLGIFALLLGLTGACFTLFLKYQSRVKYWFQAPPNIPEQIEEYLKDPDQFILEVLDKDGSPKEDSWDSVSIISSPEKERDDVLQTP

SEQ ID NO: 37 - protein, 196 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICTG

SEQ ID NO: 38 - protein, 2813 aa, Artificial Sequence
  Synthetic Polypeptide
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDVQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLCDLAPEAPPPTLPPDMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 39 - protein, 222 aa, Artificial Sequence
  Synthetic Polypeptide
CQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRITLLLMASQEPQRMSRNFVRYVQGLKKKKVIVIPVGIGPHANLKQIRLIEKQAPENKAFVLSSVDELEQQRDEIVSYLC

SEQ ID NO: 40 - protein, 344 aa, Artificial Sequence
  Synthetic Polypeptide
CGPFQQRGLFDFMLEDEASGIGPEVPDDRDFEPSLGPVCPFRCQCHLRVVQCSDLGLDKVPKDLPPDTTLLDLQNNKITEIKDGDFKNLKNLHALILVNNKISKVSPGAFTPLVKLERLYLSKNQLKELPEKMPKTLQELRAHENEITKVRKVTFNGLNQMIVIELGTNPLKSSGIENGAFQGMKKLSYIRIADTNITSIPQGLPPSLTELHLDGNKISRVDAASLKGLNNLAKLGLSFNSISAVDNGSLANTPHLRELHLDNNKLTRVPGGLAEHKYIQVVYLHNNNISVVGSSDFCPPGHNTKKASYSGVSLFSNPVQYWEIQPSTFRCVYVRSAIQLGNYK

SEQ ID NO: 41 - protein, 10 aa, Artificial Sequence
  Synthetic Polypeptide
LRELHLDNNC

SEQ ID NO: 42 - protein, 584 aa, Mus musculus
EAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 43 - protein, 608 aa, Homo Sapiens
KWVTFISLLFLFSSAYSRGVFRRDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 44 - protein, 10 aa, Artificial Sequence
  Synthetic Polypeptide
LRELHLNNNC

SEQ ID NO: 45 - protein, 193 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRI

SEQ ID NO: 46 - protein, 205 aa, Artificial Sequence
  Synthetic Polypeptide
CSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSG

SEQ ID NO: 47 - protein, 8 aa, Artificial Sequence
  Synthetic Polypeptide
GGGSGGGS

SEQ ID NO: 48 - protein, 44 aa, Artificial Sequence
  Synthetic Polypeptide
GGGSGGGSGGGSGGGSGGGSGGGSGGGSGGGSGGGSGGGSGGGS

SEQ ID NO: 49 - protein, 8 aa, Artificial Sequence
  Synthetic Polypeptide
LSGRSDNH

SEQ ID NO: 50 - protein, 7 aa, Artificial Sequence
  Synthetic Polypeptide
VPLSLYS

SEQ ID NO: 51 - protein, 6 aa, Artificial Sequence
  Synthetic Polypeptide
PLGLAG

SEQ ID NO: 52 - protein, 10 aa, Artificial Sequence
  Synthetic Polypeptide
VLVPMAMMAS

SEQ ID NO: 53 - protein, 7 aa, Artificial Sequence
  Synthetic Polypeptide
  MISC_FEATURE (1)..(1): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
  MISC_FEATURE (2)..(2): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
  MISC_FEATURE (7)..(7): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
XXQARAX

SEQ ID NO: 54 - protein, 4 aa, Artificial Sequence
  Synthetic Polypeptide
AGPR

SEQ ID NO: 55 - protein, 4 aa, Artificial Sequence
  Synthetic Polypeptide
AANL

SEQ ID NO: 56 - protein, 4 aa, Artificial Sequence
  Synthetic Polypeptide
PTNL

SEQ ID NO: 57 - protein, 8 aa, Artificial Sequence
  Synthetic Polypeptide
TSGRSANP

SEQ ID NO: 58 - protein, 39 aa, Artificial Sequence
  Synthetic Polypeptide
GASTSVDELQAEVDQLQDENYALKTKVAQLRKKVEKLSE

SEQ ID NO: 59 - protein, 38 aa, Artificial Sequence
  Synthetic Polypeptide
GASTTVAQLRERVKTLRAQNYELESEVQRLREQVAQLA

SEQ ID NO: 60 - protein, 42 aa, Artificial Sequence
  Synthetic Polypeptide
EACGASTSVDELQAEVDQLQDENYALKTKVAQLRKKVEKLSE

SEQ ID NO: 61 - protein, 41 aa, Artificial Sequence
  Synthetic Polypeptide
EACGASTTVAQLRERVKTLRAQNYELESEVQRLREQVAQLA

SEQ ID NO: 62 - protein, 40 aa, Artificial Sequence
  Synthetic Polypeptide
LEIEAAFLERENTALETRVAELRQRVQRARNRVSQYRTRY

SEQ ID NO: 63 - protein, 40 aa, Artificial Sequence
  Synthetic Polypeptide
LEIRAAFLRQRNTALRTEVAELEQEVQRLENEVSQYETRY

SEQ ID NO: 64 - protein, 45 aa, Artificial Sequence
  Synthetic Polypeptide
EACGALEIEAAFLERENTALETRVAELRQRVQRARNRVSQYRTRY

SEQ ID NO: 65 - protein, 45 aa, Artificial Sequence
  Synthetic Polypeptide
EACGALEIRAAFLRQRNTALRTEVAELEQEVQRLENEVSQYETRY

SEQ ID NO: 66 - protein, 40 aa, Artificial Sequence
  Synthetic Polypeptide
LEIRAAFLRRRNTALRTRVAELRQRVQRLRNIVSQYETRY

SEQ ID NO: 67 - protein, 40 aa, Artificial Sequence
  Synthetic Polypeptide
LEIEAAFLEQENTALETEVAELEQEVQRLENIVSQYETRY

SEQ ID NO: 68 - protein, 45 aa, Artificial Sequence
  Synthetic Polypeptide
EACGALEIRAAFLRRRNTALRTRVAELRQRVQRLRNIVSQYETRY

SEQ ID NO: 69 - protein, 45 aa, Artificial Sequence
  Synthetic Polypeptide
EACGALEIEAAFLEQENTALETEVAELEQEVQRLENIVSQYETRY

SEQ ID NO: 70 - protein, 38 aa, Artificial Sequence
  Synthetic Polypeptide
QGASTSVDELQAEVDQLEDENYALKTKVAQLRKKVEKL

SEQ ID NO: 71 - protein, 38 aa, Artificial Sequence
  Synthetic Polypeptide
QGASTTVAQLEEKVKTLRAENYELKSEVQRLEEQVAQL

SEQ ID NO: 72 - protein, 40 aa, Artificial Sequence
  Synthetic Polypeptide
EACGASTSVDELQAEVDQLEDENYALKTKVAQLRKKVEKL

SEQ ID NO: 73 - protein, 40 aa, Artificial Sequence
  Synthetic Polypeptide
EACGASTTVAQLEEKVKTLRAENYELKSEVQRLEEQVAQL

SEQ ID NO: 74 - protein, 42 aa, Artificial Sequence
  Synthetic Polypeptide
AGLTDTLQAETDQLEDKKSALQTEIANLLKEKEKLEFILAAH

SEQ ID NO: 75 - protein, 42 aa, Artificial Sequence
  Synthetic Polypeptide
AGRIARLEEKVKTLKAQNSELASTANMLREQVAQLKQKVMNY

SEQ ID NO: 76 - protein, 46 aa, Artificial Sequence
  Synthetic Polypeptide
EACGAGLTDTLQAETDQLEDKKSALQTEIANLLKEKEKLEFILAAH

SEQ ID NO: 77 - protein, 46 aa, Artificial Sequence
  Synthetic Polypeptide
EACGAGRIARLEEKVKTLKAQNSELASTANMLREQVAQLKQKVMNY

SEQ ID NO: 78 - protein, 29 aa, Artificial Sequence
  Synthetic Polypeptide
GKIAALKQKIAALKYKNAALKKKIAALKQ

SEQ ID NO: 79 - protein, 29 aa, Artificial Sequence
  Synthetic Polypeptide
GEIAALEQEIAALEKENAALEWEIAALEQ

SEQ ID NO: 80 - protein, 34 aa, Artificial Sequence
  Synthetic Polypeptide
EACGAGKIAALKQKIAALKYKNAALKKKIAALKQ

SEQ ID NO: 81 - protein, 34 aa, Artificial Sequence
  Synthetic Polypeptide
EACGAGEIAALEQEIAALEKENAALEWEIAALEQ

SEQ ID NO: 82 - protein, 15 aa, Artificial Sequence
  Synthetic Polypeptide
GLNDIFEAQKIEWHE

SEQ ID NO: 83 - protein, 26 aa, Artificial Sequence
  Synthetic Polypeptide
KRRWKKNFIAVSAANRFKKISSSGAL

SEQ ID NO: 84 - protein, 6 aa, Artificial Sequence
  Synthetic Polypeptide
EEEEEE

SEQ ID NO: 85 - protein, 8 aa, Artificial Sequence
  Synthetic Polypeptide
DYKDDDDK

SEQ ID NO: 86 - protein, 9 aa, Artificial Sequence
  Synthetic Polypeptide
YPYDVPDYA

SEQ ID NO: 87 - protein, 6 aa, Artificial Sequence
  Synthetic Polypeptide
HHHHHH

SEQ ID NO: 88 - protein, 10 aa, Artificial Sequence
  Synthetic Polypeptide
EQKLISEEDL

SEQ ID NO: 89 - protein, 18 aa, Artificial Sequence
  Synthetic Polypeptide
TKENPRSNQEESYDDNES

SEQ ID NO: 90 - protein, 15 aa, Artificial Sequence
  Synthetic Polypeptide
KETAAAKFERQHMDS

SEQ ID NO: 91 - protein, 38 aa, Artificial Sequence
  Synthetic Polypeptide
MDEKTTGWRGGHVVEGLAGELEQLRARLEHHPQGQREP

SEQ ID NO: 92 - protein, 13 aa, Artificial Sequence
  Synthetic Polypeptide
SLAELLNAGLGGS

SEQ ID NO: 93 - protein, 8 aa, Artificial Sequence
  Synthetic Polypeptide
TQDPSRVG

SEQ ID NO: 94 - protein, 8 aa, Artificial Sequence
  Synthetic Polypeptide
WSHPQFEK

SEQ ID NO: 95 - protein, 6 aa, Artificial Sequence
  Synthetic Polypeptide
CCPGCC

SEQ ID NO: 96 - protein, 14 aa, Artificial Sequence
  Synthetic Polypeptide
GKPIPNPLLGLDST

SEQ ID NO: 97 - protein, 11 aa, Artificial Sequence
  Synthetic Polypeptide
YTDIEMNRLGK

SEQ ID NO: 98 - protein, 8 aa, Artificial Sequence
  Synthetic Polypeptide
DLYDDDDK

SEQ ID NO: 99 - protein, 16 aa, Artificial Sequence
  Synthetic Polypeptide
TDKDMTITFTNKKDAE

SEQ ID NO: 100 - protein, 13 aa, Artificial Sequence
  Synthetic Polypeptide
AHIVMVDAYKPTK

SEQ ID NO: 101 - protein, 12 aa, Artificial Sequence
  Synthetic Polypeptide
KLGDIEFIKVNK

SEQ ID NO: 102 - protein, 13 aa, Artificial Sequence
  Synthetic Polypeptide
GAPVPYPDPLEPR

SEQ ID NO: 103 - protein, 41 aa, Artificial Sequence
  Synthetic Polypeptide
HPVGLLARVPLSYSGHPVGLLARVPLSYSGSGLLSGRSDNH

SEQ ID NO: 104 - protein, 21 aa, Artificial Sequence
  Synthetic Polypeptide
PLGVRGKPLGVRGKPLGVRGK

SEQ ID NO: 105 - protein, 42 aa, Artificial Sequence
  Synthetic Polypeptide
GERGPPGPQGAARGFPGTPGLGERGPPGPQGAARGFPGTPGL

SEQ ID NO: 106 - protein, 63 aa, Artificial Sequence
  Synthetic Polypeptide
PLGVRGKPLGVRGKPLGVRGKGERGPPGPQGAARGFPGTPGLGERGPPGPQGAARGFPGTPGL

SEQ ID NO: 107 - protein, 14 aa, Artificial Sequence
  Synthetic Polypeptide
PLGVRGKPLGVRGK

SEQ ID NO: 108 - protein, 97 aa, Artificial Sequence
  Synthetic Polypeptide
HPVGLLARVPLSYSGHPVGLLARVPLSYSGSGLLSGRSDNHPLGVRGKPLGVRGKGERGPPGPQGAARGFPGTPGLGERGPPGPQGAARGFPGTPGL

SEQ ID NO: 109 - protein, 8 aa, Artificial Sequence
  Synthetic Polypeptide
YLGRSYKV

SEQ ID NO: 110 - protein, 8 aa, Artificial Sequence
  Synthetic Polypeptide
TSQVNGLN

SEQ ID NO: 111 - protein, 8 aa, Artificial Sequence
  Synthetic Polypeptide
APPPVVLL

SEQ ID NO: 112 - protein, 5 aa, Artificial Sequence
  Synthetic Polypeptide
GGGGF

SEQ ID NO: 113 - protein, 8 aa, Artificial Sequence
  Synthetic Polypeptide
TPEHVVPY

SEQ ID NO: 114 - protein, 9 aa, Artificial Sequence
  Synthetic Polypeptide
PRSFFRLGK

SEQ ID NO: 115 - protein, 8 aa, Artificial Sequence
  Synthetic Polypeptide
PQGRIVGG

SEQ ID NO: 116 - protein, 8 aa, Artificial Sequence
  Synthetic Polypeptide
EVLLSWAV

SEQ ID NO: 117 - protein, 8 aa, Artificial Sequence
  Synthetic Polypeptide
PVSLSYRC

SEQ ID NO: 118 - protein, 8 aa, Artificial Sequence
  Synthetic Polypeptide
LRELHLDN

SEQ ID NO: 119 - protein, 4 aa, Artificial Sequence
  Synthetic Polypeptide
AAPV

SEQ ID NO: 120 - protein, 8 aa, Artificial Sequence
  Synthetic Polypeptide
IPENFFGV

SEQ ID NO: 121 - protein, 8 aa, Artificial Sequence
  Synthetic Polypeptide
PRFKIIGG

SEQ ID NO: 122 - protein, 8 aa, Artificial Sequence
  Synthetic Polypeptide
MLEDEASG

SEQ ID NO: 123 - protein, 8 aa, Artificial Sequence
  Synthetic Polypeptide
KQSRKFVP

SEQ ID NO: 124 - protein, 8 aa, Artificial Sequence
  Synthetic Polypeptide
RQARVVGG

SEQ ID NO: 125 - protein, 7 aa, Artificial Sequence
  Synthetic Polypeptide
GKAFRRL

SEQ ID NO: 126 - protein, 8 aa, Artificial Sequence
  Synthetic Polypeptide
GPLGLWAQ

SEQ ID NO: 127 - protein, 8 aa, Artificial Sequence
  Synthetic Polypeptide
SLGRKIQI

SEQ ID NO: 128 - protein, 5 aa, Artificial Sequence
  Synthetic Polypeptide
PVSLR

SEQ ID NO: 129 - protein, 8 aa, Artificial Sequence
  Synthetic Polypeptide
AAATSIAM

SEQ ID NO: 130 - protein, 8 aa, Artificial Sequence
  Synthetic Polypeptide
AAGAMFLE

SEQ ID NO: 131 - protein, 8 aa, Artificial Sequence
  Synthetic Polypeptide
EAAAATSI

SEQ ID NO: 132 - protein, 5 aa, Artificial Sequence
  Synthetic Polypeptide
PRHLR

SEQ ID NO: 133 - protein, 6 aa, Artificial Sequence
  Synthetic Polypeptide
PRGLRK

SEQ ID NO: 134 - protein, 8 aa, Artificial Sequence
  Synthetic Polypeptide
VPLSLYSG

SEQ ID NO: 135 - protein, 6 aa, Artificial Sequence
  Synthetic Polypeptide
PRGLRP

SEQ ID NO: 136 - protein, 6 aa, Artificial Sequence
  Synthetic Polypeptide
PRHLRN

SEQ ID NO: 137 - protein, 6 aa, Artificial Sequence
  Synthetic Polypeptide
PRWLRS

SEQ ID NO: 138 - protein, 8 aa, Artificial Sequence
  Synthetic Polypeptide
HPVGLLAR

SEQ ID NO: 139 - protein, 8 aa, Artificial Sequence
  Synthetic Polypeptide
KGPLGVRG

SEQ ID NO: 140 - protein, 5 aa, Artificial Sequence
  Synthetic Polypeptide
CGLDD

SEQ ID NO: 141 - protein, 8 aa, Artificial Sequence
  Synthetic Polypeptide
STAVIVSA

SEQ ID NO: 142 - protein, 8 aa, Artificial Sequence
  Synthetic Polypeptide
GPLGIAGQ

SEQ ID NO: 143 - protein, 6 aa, Artificial Sequence
  Synthetic Polypeptide
PLGLWA

SEQ ID NO: 144 - protein, 8 aa, Artificial Sequence
  Synthetic Polypeptide
GPLGMLSQ

SEQ ID NO: 145 - protein, 7 aa, Artificial Sequence
  Synthetic Polypeptide
PLGVRGK

SEQ ID NO: 146 - protein, 8 aa, Artificial Sequence
  Synthetic Polypeptide
GPQGIWGQ

SEQ ID NO: 147 - protein, 7 aa, Artificial Sequence
  Synthetic Polypeptide
AIPVSLR

SEQ ID NO: 148 - protein, 8 aa, Artificial Sequence
  Synthetic Polypeptide
GPLGLARK

SEQ ID NO: 149 - protein, 7 aa, Artificial Sequence
  Synthetic Polypeptide
PQGIAMG

SEQ ID NO: 150 - protein, 8 aa, Artificial Sequence
  Synthetic Polypeptide
VASSSTAV

SEQ ID NO: 151 - protein, 4 aa, Artificial Sequence
  Synthetic Polypeptide
PLGL

SEQ ID NO: 152 - protein, 8 aa, Artificial Sequence
  Synthetic Polypeptide
RPLALWRS

SEQ ID NO: 153 - protein, 8 aa, Artificial Sequence
  Synthetic Polypeptide
NKSRLGLG

SEQ ID NO: 154 - protein, 9 aa, Artificial Sequence
  Synthetic Polypeptide
GPQGIAGQR

SEQ ID NO: 155 - protein, 6 aa, Artificial Sequence
  Synthetic Polypeptide
PVGLIG

SEQ ID NO: 156 - protein, 8 aa, Artificial Sequence
  Synthetic Polypeptide
KPVSLSYR

SEQ ID NO: 157 - protein, 8 aa, Artificial Sequence
  Synthetic Polypeptide
QPVGINTS

SEQ ID NO: 158 - protein, 6 aa, Artificial Sequence
  Synthetic Polypeptide
PLGMTS

SEQ ID NO: 159 - protein, 5 aa, Artificial Sequence
  Synthetic Polypeptide
PRALM

SEQ ID NO: 160 - protein, 6 aa, Artificial Sequence
  Synthetic Polypeptide
GPLPLR

SEQ ID NO: 161 - protein, 8 aa, Artificial Sequence
  Synthetic Polypeptide
KQLRVVNG

SEQ ID NO: 162 - protein, 6 aa, Artificial Sequence
  Synthetic Polypeptide
PLGLYA

SEQ ID NO: 163 - protein, 7 aa, Artificial Sequence
  Synthetic Polypeptide
HSSKLQL

SEQ ID NO: 164 - protein, 5 aa, Artificial Sequence
  Synthetic Polypeptide
SSKYQ

SEQ ID NO: 165 - protein, 8 aa, Artificial Sequence
  Synthetic Polypeptide
GPLGVRGK

SEQ ID NO: 166 - protein, 6 aa, Artificial Sequence
  Synthetic Polypeptide
PRGMAS

SEQ ID NO: 167 - protein, 9 aa, Artificial Sequence
  Synthetic Polypeptide
AAALGNVAP

SEQ ID NO: 168 - protein, 9 aa, Artificial Sequence
  Synthetic Polypeptide
GGSGRSANA

SEQ ID NO: 169 - protein, 6 aa, Artificial Sequence
  Synthetic Polypeptide
PLGVRG

SEQ ID NO: 170 - protein, 8 aa, Artificial Sequence
  Synthetic Polypeptide
SGRSANAK

SEQ ID NO: 171 - protein, 9 aa, Artificial Sequence
  Synthetic Polypeptide
GPLGLWAGG

SEQ ID NO: 172 - protein, 5 aa, Artificial Sequence
  Synthetic Polypeptide
SGRSA

SEQ ID NO: 173 - protein, 6 aa, Artificial Sequence
  Synthetic Polypeptide
PLGLYL

SEQ ID NO: 174 - protein, 8 aa, Artificial Sequence
  Synthetic Polypeptide
SRRRVNSL

SEQ ID NO: 175 - protein, 5 aa, Artificial Sequence
  Synthetic Polypeptide
GSTFF

SEQ ID NO: 176 - protein, 8 aa, Artificial Sequence
  Synthetic Polypeptide
CPGRVVGG

SEQ ID NO: 177 - protein, 5 aa, Artificial Sequence
  Synthetic Polypeptide
GTQFF

SEQ ID NO: 178 - protein, 6 aa, Artificial Sequence
  Synthetic Polypeptide
PQGLAG

SEQ ID NO: 179 - protein, 5 aa, Artificial Sequence
  Synthetic Polypeptide
QVVAG

SEQ ID NO: 180 - protein, 8 aa, Artificial Sequence
  Synthetic Polypeptide
TYSRSRYL

SEQ ID NO: 181 - protein, 8 aa, Artificial Sequence
  Synthetic Polypeptide
NSGRAVTY

SEQ ID NO: 182 - protein, 8 aa, Artificial Sequence
  Synthetic Polypeptide
GPQGARGQ

SEQ ID NO: 183 - protein, 8 aa, Artificial Sequence
  Synthetic Polypeptide
PSSRRRVN

SEQ ID NO: 184 - protein, 7 aa, Artificial Sequence
  Synthetic Polypeptide
PLGLYAL

SEQ ID NO: 185 - protein, 8 aa, Artificial Sequence
  Synthetic Polypeptide
PMKRLTLG

SEQ ID NO: 186 - protein, 8 aa, Artificial Sequence
  Synthetic Polypeptide
DDDKIVGG

SEQ ID NO: 187 - protein, 8 aa, Artificial Sequence
  Synthetic Polypeptide
HLVEALYL

SEQ ID NO: 188 - protein, 8 aa, Artificial Sequence
  Synthetic Polypeptide
EVDLLIGS

SEQ ID NO: 189 - protein, 8 aa, Artificial Sequence
  Synthetic Polypeptide
AVRWLLTA

SEQ ID NO: 190 - protein, 5 aa, Artificial Sequence
  Synthetic Polypeptide
GGGRR

SEQ ID NO: 191 - protein, 4 aa, Artificial Sequence
  Synthetic Polypeptide
GGGS

SEQ ID NO: 192 - protein, 241 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQA

SEQ ID NO: 193 - protein, 5 aa, Artificial Sequence
  Synthetic Polypeptide
HHHHH

SEQ ID NO: 194 - protein, 21 aa, Artificial Sequence
  Synthetic Polypeptide
GERGPPGPQGAARGFPGTPGL

SEQ ID NO: 195 - protein, 211 aa, Homo Sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPE

SEQ ID NO: 196 - protein, 453 aa, Homo Sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGSGGGSGGGSGGGSGGGSGGGSGGGSGGGSGGGSGGGSGGGSGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 197 - protein, 459 aa, Homo Sapiens
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEHPVGLLARVPLSLYSGHPVGLLARVPLSLYSGSGLLSGRSDNHGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 198 - protein, 483 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGHPVGLLARVPLSYSGSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 199 - protein, 463 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAPLGVRGKPLGVRGKPLGVRGKGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 200 - protein, 484 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGERGPPGPQGAARGFPGTPGLGERGPPGPQGAARGFPGTPGLGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 201 - protein, 505 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAPLGVRGKPLGVRGKPLGVRGKGERGPPGPQGAARGFPGTPGLGERGPPGPQGAARGFPGTPGLGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 202 - protein, 497 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGHPVGLLARVPLSYSGSGLLSGRSDNHPLGVRGKPLGVRGKGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 203 - protein, 539 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGHPVGLLARVPLSYSGSGLLSGRSDNHPLGVRGKPLGVRGKGERGPPGPQGAARGFPGTPGLGERGPPGPQGAARGFPGTPGLGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 204 - protein, 660 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 205 - protein, 675 aa, Artificial Sequence
  Synthetic Polypeptide
CSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRIGGGSGGGSCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGHPVGLLARVPLSYSGSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 206 - protein, 509 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSYSGHPVGLLARVPLSYSGSGLLSGRSDNHISSGLLSGRSDNHISSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 207 - protein, 482 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSGGGSGGGSPLGVRGKGGGSGGGSGGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 208 - protein, 475 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSHPVGLLARHPVGLLARHPVGLLARGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 209 - protein, 484 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSSGLLSGRSDNHSGLLSGRSDNHSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 210 - protein, 478 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSHPVGLLARVPLSLYSGSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 211 - protein, 486 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAHPVGLLARVPLSLYSGHPVGLLARVPLSLYSGSGLLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 212 - protein, 479 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSVPLSLYSGVPLSLYSGVPLSLYSGGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 213 - protein, 483 aa, Artificial Sequence
  Synthetic Polypeptide
QLGASGPGDGCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSVPLSLYSGGGGSGGGSLSGRSDNHLSGRSDNHGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 214 - protein, 457 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGSGGGSVPLSLYSGGGGSGGGSLSGRSDNHLSGRSDNHGGGSGGGSGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 215 - protein, 452 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGSGGGSVPLSLYSGVPLSLYSGVPLSLYSGGGGSGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 216 - protein, 451 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGSGGGSHPVGLLARVPLSLYSGSGLLSGRSDNHGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 217 - protein, 472 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEPLGVRGKPLGVRGKGERGPPGPQGAARGFPGTPGLGERGPPGPQGAARGFPGTPGLGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 218 - protein, 471 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEHPVGLLARVPLSYSGHPVGLLARVPLSYSGSGLLSGRSDNHPLGVRGKPLGVRGKGGGSGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 219 - protein, 43 aa, Artificial Sequence
  Synthetic Polypeptide
HPVGLLARVPLSLYSGHPVGLLARVPLSLYSGSGLLSGRSDNH

SEQ ID NO: 220 - protein, 51 aa, Artificial Sequence
  Synthetic Polypeptide
HPVGLLARVPLSLYSGHPVGLLARVPLSLYSGSGLLSGRSDNHGGGSGGGS

SEQ ID NO: 221 - protein, 26 aa, Artificial Sequence
  Synthetic Polypeptide
HPVGLLARVPLSYSGSGLLSGRSDNH

SEQ ID NO: 222 - protein, 67 aa, Artificial Sequence
  Synthetic Polypeptide
HPVGLLARVPLSYSGHPVGLLARVPLSYSGSGLLSGRSDNHISSGLLSGRSDNHISSGLLSGRSDNH

SEQ ID NO: 223 - protein, 47 aa, Artificial Sequence
  Synthetic Polypeptide
GGGSGGGSGGGSGGGSPLGVRGKGGGSGGGSGGGSGGGSGGGSGGGS

SEQ ID NO: 224 - protein, 24 aa, Artificial Sequence
  Synthetic Polypeptide
HPVGLLARHPVGLLARHPVGLLAR

SEQ ID NO: 225 - protein, 49 aa, Artificial Sequence
  Synthetic Polypeptide
HPVGLLARVPLSYSGHPVGLLARVPLSYSGSGLLSGRSDNHGGGSGGGS

SEQ ID NO: 226 - protein, 29 aa, Artificial Sequence
  Synthetic Polypeptide
PLGVRGKPLGVRGKPLGVRGKGGGSGGGS

SEQ ID NO: 227 - protein, 50 aa, Artificial Sequence
  Synthetic Polypeptide
GERGPPGPQGAARGFPGTPGLGERGPPGPQGAARGFPGTPGLGGGSGGGS

SEQ ID NO: 228 - protein, 71 aa, Artificial Sequence
  Synthetic Polypeptide
PLGVRGKPLGVRGKPLGVRGKGERGPPGPQGAARGFPGTPGLGERGPPGPQGAARGFPGTPGLGGGSGGGS

SEQ ID NO: 229 - protein, 22 aa, Artificial Sequence
  Synthetic Polypeptide
PLGVRGKPLGVRGKGGGSGGGS

SEQ ID NO: 230 - protein, 105 aa, Artificial Sequence
  Synthetic Polypeptide
HPVGLLARVPLSYSGHPVGLLARVPLSYSGSGLLSGRSDNHPLGVRGKPLGVRGKGERGPPGPQGAARGFPGTPGLGERGPPGPQGAARGFPGTPGLGGGSGGGS

SEQ ID NO: 231 - protein, 34 aa, Artificial Sequence
  Synthetic Polypeptide
HPVGLLARVPLSYSGSGLLSGRSDNHGGGSGGGS

SEQ ID NO: 232 - protein, 75 aa, Artificial Sequence
  Synthetic Polypeptide
HPVGLLARVPLSYSGHPVGLLARVPLSYSGSGLLSGRSDNHISSGLLSGRSDNHISSGLLSGRSDNHGGGSGGGS

SEQ ID NO: 233 - protein, 40 aa, Artificial Sequence
  Synthetic Polypeptide
GGGSGGGSHPVGLLARHPVGLLARHPVGLLARGGGSGGGS

SEQ ID NO: 234 - protein, 33 aa, Artificial Sequence
  Synthetic Polypeptide
SGLLSGRSDNHSGLLSGRSDNHSGLLSGRSDNH

SEQ ID NO: 235 - protein, 49 aa, Artificial Sequence
  Synthetic Polypeptide
GGGSGGGSSGLLSGRSDNHSGLLSGRSDNHSGLLSGRSDNHGGGSGGGS

SEQ ID NO: 236 - protein, 27 aa, Artificial Sequence
  Synthetic Polypeptide
HPVGLLARVPLSLYSGSGLLSGRSDNH

SEQ ID NO: 237 - protein, 43 aa, Artificial Sequence
  Synthetic Polypeptide
GGGSGGGSHPVGLLARVPLSLYSGSGLLSGRSDNHGGGSGGGS

SEQ ID NO: 238 - protein, 23 aa, Artificial Sequence
  Synthetic Polypeptide
VPLSLYSGVPLSLYSGVPLSLYS

SEQ ID NO: 239 - protein, 44 aa, Artificial Sequence
  Synthetic Polypeptide
GGGSGGGSVPLSLYSGVPLSLYSGVPLSLYSGGGGSGGGSGGGS

SEQ ID NO: 240 - protein, 32 aa, Artificial Sequence
  Synthetic Polypeptide
VPLSLYSGGGGSGGGSLSGRSDNHLSGRSDNH

SEQ ID NO: 241 - protein, 48 aa, Artificial Sequence
  Synthetic Polypeptide
GGGSGGGSVPLSLYSGGGGSGGGSLSGRSDNHLSGRSDNHGGGSGGGS

SEQ ID NO: 242 - protein, 49 aa, Artificial Sequence
  Synthetic Polypeptide
GGGSGGGSVPLSLYSGGGGSGGGSLSGRSDNHLSGRSDNHGGGSGGGSG

SEQ ID NO: 243 - protein, 56 aa, Artificial Sequence
  Synthetic Polypeptide
PLGVRGKPLGVRGKGERGPPGPQGAARGFPGTPGLGERGPPGPQGAARGFPGTPGL

SEQ ID NO: 244 - protein, 64 aa, Artificial Sequence
  Synthetic Polypeptide
PLGVRGKPLGVRGKGERGPPGPQGAARGFPGTPGLGERGPPGPQGAARGFPGTPGLGGGSGGGS

SEQ ID NO: 245 - protein, 55 aa, Artificial Sequence
  Synthetic Polypeptide
HPVGLLARVPLSYSGHPVGLLARVPLSYSGSGLLSGRSDNHPLGVRGKPLGVRGK

SEQ ID NO: 246 - protein, 63 aa, Artificial Sequence
  Synthetic Polypeptide
HPVGLLARVPLSYSGHPVGLLARVPLSYSGSGLLSGRSDNHPLGVRGKPLGVRGKGGGSGGGS

SEQ ID NO: 247 - protein, 484 aa, Artificial Sequence
  Synthetic Polypeptide
QLGASGPGDCCVEKTSFPEGASGSPLGPRNLSCYRVSKTDYECSWQYDGPEDNVSHVLWCCFVPPNHTHTGQERCRYFSSGPDRTVQFWEQDGIPVLSKVNFWVESRLGNRTMKSQKISQYLYNWTKTTPPLGHIKVSQSHRQLRMDWNVSEEAGAEVQFRRRMPTTNWTLGDCGPQVNSGSGVLGDIRGSMSESCLCPSENMAQEIQIRRRRRLSSGAPGGPWSDWSMPVCVPPEVLPQAGGGSGGGSGGGSGGGSGGGSGGGSGGGSGGGSGGGSGGGSGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSAHHHHHH

SEQ ID NO: 248 - protein, 459 aa, Artificial Sequence
  Synthetic Polypeptide
CRTSECCFQDPPYPDADSGSASGPRDLRCYRISSDRYECSWQYEGPTAGVSHFLRCCLSSGRCCYFAAGSATRLQFSDQAGVSVLYTVTLWVESWARNQTEKSPEVTLQLYNSVKYEPPLGDIKVSKLAGQLRMEWETPDNQVGAEVQFRHRTPSSPWKLGDCGPQDDDTESCLCPLEMNVAQEFQLRRRQLGSQGSSWSKWSSPVCVPPEGGGSGGGSGGGSGGGSGGGSGGGSGGGSGGGSGGGSGGGSGGGSGRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASHHHHHH

SEQ ID NO: 249 - protein, 7 aa, Artificial Sequence
  Synthetic Polypeptide
  misc_feature (1)..(1): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
  misc_feature (2)..(2): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
  misc_feature (7)..(7): X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine
XXQARVX

(19) United States
(12) Patent Application Publication
(10) Pub. No.: US 2024/0228586 A1
(43) Pub. Date: Jul. 11, 2024

(54) COMPOSITIONS AND METHODS COMPRISING PROTEASE-ACTIVATED THERAPEUTIC AGENTS

(71) Applicant: THE UNIVERSITY OF CHICAGO, Chicago, IL (US)

(72) Inventors: Jeffrey HUBBELL, Chicago, IL (US); Jun ISHIHARA, Chicago, IL (US); Juan MENDOZA, Chicago, IL (US); Aslan MANSUROV, Chicago, IL (US)

(73) Assignee: THE UNIVERSITY OF CHICAGO, Chicago, IL (US)

(21) Appl. No.: 18/559,180

(22) PCT Filed: May 5, 2022
(86) PCT No.: PCT/US2022/072121

§ 371 Date: Nov. 6, 2023

Related U.S. Application Data
(60) Provisional application No. 63/313,087, filed on Feb. 23, 2022.
(60) Provisional application No. 63/185,941, filed on May 7, 2021.

(51) Int. Cl. C07K 14/715; A61K 38/17; A61K 38/20; A61K 38/21; A61K 39/395; A61K 47/64; A61K 47/68; A61P 35/00; C07K 14/47; C07K 14/54; C07K 14/55; C07K 14/57

(52) U.S. Cl. CPC C07K 14/7155; A61K 38/1709; A61K 38/1793; A61K 38/2013; A61K 38/208; A61K 38/217; A61K 39/395; A61K 47/643; A61K 47/6843; A61P 35/00; C07K 14/47; C07K 14/5434; C07K 14/55; C07K 14/57; C07K 14/7156; C07K 2319/50

(57) ABSTRACT

The disclosure relates to the engineering of collagen-binding modification of masked therapeutic agents comprising one or more tumor-associated protease cleavage sites. Upon exposure to tumor-associated proteases in the tumor microenvironment, the polypeptide is cleaved, which unmasks the therapeutic agent, reducing off-target side effects and toxicity associated with systemic administration. Accordingly, aspects of the disclosure relate to a polypeptide comprising a therapeutic agent linked to a masking agent through a linker, wherein the linker comprises one or more tumor-associated protease cleavage sites, and wherein the masking agent blocks the association of the therapeutic agent to its therapeutic target, and further wherein the polypeptide is operatively linked to a collagen binding domain or a tumor-targeting agent.

### CROSS-REFERENCE FOR RELATED APPLICATIONS

This application claims the benefit of priority of U.S. Provisional Patent Application No. 63/185,941 filed May 7, 2021, and U.S. Provisional Patent Application No. 63/313,087 filed Feb. 23, 2022, which are hereby incorporated by reference in their entirety.

### BACKGROUND OF THE INVENTION

### I. Field of the Invention

This invention relates to the field of treatment of disease and, in particular, to the treatment of cancer.

### II. Background

Cytokine cancer immunotherapy using interleukin (IL)-12 has shown strong antitumor efficacy in both mouse and in human. However, due to its severe toxicity, some IL12 clinical trials have been terminated or unsuccessful. IL12 has not been approved to use in the clinic to date. Immunotherapies serve to activate immune responses, and as such, side-effects typically result from drug action in healthy organs. There is a need in the art for strategies to reduce the toxicity of therapeutic treatments.

### SUMMARY OF INVENTION

The disclosure relates to the engineering of masked therapeutic agents comprising one or more tumor-associated protease cleavage sites. Upon exposure to tumor-associated proteases in the tumor microenvironment, the polypeptide is cleaved, which unmasks the therapeutic agent, reducing off-target side effects and toxicity associated with systemic administration. Accordingly, aspects of the disclosure relate to a polypeptide comprising a cytokine linked to a masking agent through a linker, wherein the linker comprises at least 2 protease cleavage sites of SEQ ID NO:138 and at least two protease cleavage sites of SEQ ID NO:134, and wherein the masking agent comprises a cytokine receptor polypeptide or fragment thereof that specifically binds to the cytokine. Further aspects relate to a polypeptide comprising a cytokine linked to a masking agent through a linker, wherein the linker comprises an amino acid sequence with at least 80% sequence identity to one of SEQ ID NOS:48, 103-108, or 219-246, and wherein the masking agent comprises a cytokine receptor polypeptide or fragment thereof that specifically binds to the cytokine. Also described is a polypeptide comprising a cytokine linked to a masking agent through a linker, wherein the linker comprises a glycine-serine linker; and wherein the masking agent comprises a cytokine receptor polypeptide or fragment thereof that specifically binds to the cytokine.

The linker may comprise an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS:48, 103-108, or 219-246. In some aspects, the linker comprises or consists of the amino acid sequence of one of SEQ ID NOS:48, 103-108, or 219-246. In some aspects, the linker comprises SEQ ID NO:48.

Yet further aspects relate to a polypeptide comprising the amino acid sequence of one of SEQ ID NOS: 197-218, 247, or 248 or an amino acid sequence with at least 80% sequence identity to one of SEQ ID NOS: 197-218, 247, or 248. In some aspects, the polypeptide comprises or consists of an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to one of SEQ ID NOS: 197-218, 247-248.

Further aspects of the disclosure relate to a polypeptide comprising a cytokine linked to a masking agent through a linker, wherein the linker comprises one or more tumor-associated protease cleavage sites, and wherein the masking agent comprises a cytokine receptor polypeptide or fragment thereof that specifically binds to the cytokine. A masking agent refers to a molecule that blocks the association of a therapeutic agent with at least one binding partner. In some aspects, the therapeutic agent comprises an antibody, and the binding partner comprises an antigen. In some aspects, the therapeutic agent comprises a cytokine and the binding partner comprises a receptor polypeptide.

Further aspects relate to a composition comprising the polypeptides of the disclosure. Yet further aspects relate to nucleic acids encoding polypeptides of the disclosure and host cells comprising nucleic acids and/or polypeptides of the disclosure. Also provided are methods for making a polypeptide comprising expressing a nucleic acid of the disclosure in a host cell and isolating the expressed polypeptide. Further aspects relate to a method for treating cancer comprising administering a polypeptide or composition of the disclosure to a subject in need thereof, such as one that has cancer.

The cytokine may comprise interleukin-12 (IL12) and the masking agent may comprise interleukin 12 receptor (IL12R) polypeptide or an IL12-binding fragment thereof. In some aspects, the IL12 comprises one or both of the p35 and p40 subunits. In some aspects, the IL12 comprises the p35 and p40 subunits linked through a disulfide bond. In some aspects, the IL12 comprises the p35 and p40 subunits linked through a peptide linker. In some aspects, the IL12R polypeptide or fragment comprises interleukin 12 receptor beta 1 (IL12Rβ1), or a fragment thereof. The IL12 may comprise a polypeptide of SEQ ID NO:3 or a polypeptide having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:3. The IL12R polypeptide or fragment may comprise interleukin 12 receptor beta 2 (IL12Rβ2), or a fragment thereof. The IL12Rβ1 polypeptide may comprise human FNI-II domain of IL-12Rβ1. In some aspects, the IL12Rβ1 polypeptide comprises a polypeptide of SEQ ID NO: 195 or a polypeptide having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 195. In some aspects, the polypeptide has or has at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO: 195 and wherein the polypeptide binds to IL12. In some aspects, the polypeptide comprises SEQ ID NO:197 or a polypeptide having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:197.

In some aspects, the masking agent is fused to the N-terminus of the p35 subunit of IL12, and wherein the linker comprising the tumor-associated protease cleavage site is between the masking agent and the p35 subunit of IL12. In some aspects, the masking agent is fused to the C-terminus of the p35 subunit of IL12, and wherein the linker comprising the tumor-associated protease cleavage site is between the masking agent and the p35 subunit of IL12. In some aspects, the masking agent is fused to the C-terminus of the p40 subunit of IL12, and wherein the linker is between the masking agent and the p40 subunit of IL12. In some aspects, the masking agent is fused to the N-terminus of the p40 subunit of IL12, and wherein the linker is between the masking agent and the p40 subunit of IL12. In some aspects, the cytokine comprises interleukin-2 (IL-2) and the masking agent comprises interleukin 2 receptor alpha subunit (IL-2Rα), interleukin 2 receptor beta subunit (IL-2Rβ), interleukin 2 receptor gamma subunit (IL-2Rγ), fragments, or combinations of fragments thereof. In some aspects, the cytokine comprises the amino acid sequence of SEQ ID NO:23 or an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:23. In some aspects, the masking agent comprises the amino acid sequence of SEQ ID NO:27, 29, 31 or a fragment or combination of SEQ ID NO:27, 29, or 31. In some aspects, the masking agent comprises an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:27, 29, and/or 31.

In some aspects, the cytokine comprises interferon-gamma (IFNγ) and the masking agent comprises interferon-gamma receptor 1 (IFNγR1), interferon-gamma receptor 2 (IFNγR2), fragments, or combinations of fragments thereof. In some aspects, the cytokine comprises the amino acid sequence of SEQ ID NO:26 or an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:26. In some aspects, the masking agent comprises the amino acid sequence of SEQ ID NO:33, 35 or a fragment or combination of SEQ ID NO:33 and/or 35. In some aspects, the masking agent comprises an amino acid sequence having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:33 and/or 35.

In some aspects, the polypeptide comprises at least four tumor-associated protease cleavage sites. In some aspects, the polypeptide comprises at least, at most, or exactly 4, 5, 6, 7, 8, 9, 10, 11, or 12 tumor-associated protease cleavage sites, or any range derivable therein. In some aspects, the linker further comprises at least one serine protease sensitive cleavage site. The serine protease sensitive cleavage site may comprise a cleavage site of SEQ ID NO:47 or SEQ ID NO:3. In some aspects, the linker comprises SEQ ID NO:219 or a polypeptide having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:219. In some aspects, the linker comprises SEQ ID NO:220 or a polypeptide having or having at least 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:220.

In some aspects, the linker comprises or further comprises (GGGS)n, wherein n is 1-4. In some aspects, n is equal to 2.

In some aspects, the tumor-associated protease cleavage site comprises at least one tumor associated protease cleavage site described herein. In some aspects, the tumor-associated cleavage site comprises a cleavage site with an amino acid of one of SEQ ID NOS:13, 14, 49, 51, 55, 109-190. In some aspects, the polypeptide comprises at least two different tumor-associated protease cleavage site. In some aspects, the polypeptide comprises at least 2, 3, or 4 different tumor-associated protease cleavage sites, or any range derivable therein. In some aspects, the polypeptide comprises at least 2 of the same tumor-associated protease cleavage sites. In some aspects, the polypeptide comprises at least 2, 3, 4, 5, 6, 7, or 8 of the same protease cleavage sites, or any range derivable therein. In aspects which comprise more than one protease cleavage site, the protease cleavage sites may be adjacent or may have intervening amino acids. In some aspects, at least, at most, or exactly 0, 1, 2, 3, 4, 5, 6, 7, 8, 9, 10, 11, 12, 13, 14, 15, 16, 17, 18, 19, or 20 amino acids, or any range derivable therein, separate one tumor-associated protease cleavage site from another tumor-associated protease cleavage site.

In some aspects, the cytokine comprises an anti-inflammatory cytokine. In some aspects, the cytokine comprises a pro-inflammatory cytokine.

In some aspects, the polypeptide is conjugated to a tumor targeting agent. In some aspects, the tumor targeting agent comprises an antibody or an antigen-binding fragment thereof. In some aspects, the antibody or antigen-binding fragment comprises a stroma targeting antibody or stroma-binding fragment thereof. In some aspects, the antibody or binding fragment specifically binds to fibronectin, alternatively spliced domains of fibronectin, collagens, tenascins, periostins, syndecans, proteoglycans, or a tumor stroma cell-specific antigen. In some aspects, the antibody or binding fragment specifically binds toe extra domain A (EDA) or extra domain B (EDB) of fibronectin. In some aspects, the tumor targeting agent comprises a Fab that specifically binds to an alternatively spliced domain of fibronectin comprising extra domain A (EDA). In some aspects, the tumor targeting agent comprises an antibody or antigen binding fragment thereof that specifically binds to a tumor-associated antigen. Other tumor targeting agents include those recited in US20140294723A1, WO2001062298A2, WO1997045544A1, WO2006119897A2, WO2006050834A2, WO2008120101A2, WO2010078916A1, which are herein incorporated by reference.

In some aspects, the tumor targeting agent comprises a collagen binding domain. In some aspects, the polypeptide comprises at least two collagen binding domains. In some aspects, the polypeptide comprises at least 2, 3, 4, 5, or 6 collagen binding domains. In some aspects, the polypeptide comprises a collagen binding domain from decorin or von Willebrand factor (VWF). In some aspects, the collagen binding domain comprises a polypeptide comprising SEQ ID NO:1 or an amino acid sequence having or having at least 78, 79, 80, 81, 82, 83, 84, 85, 86, 87, 88, 89, 90, 91, 92, 93, 94, 95, 96, 97, 98, 99, or 100% sequence identity to SEQ ID NO:1.

In some aspects, the polypeptide further comprises a serum protein conjugated to the polypeptide. In some aspects, the serum protein is conjugated to the polypeptide through a peptide bond. In some aspects, the serum protein comprises albumin or a fragment thereof. In some aspects, the serum protein is at least 40, 45, 50, 55, 60, 65, 70, or 75 kDa (or any range derivable therein).

In some aspects, the polypeptide comprises a second linker. In some aspects, the second linker comprises glycine and serine amino acid residues. In some aspects, the polypeptide comprises a third, fourth, or fifth linker. In some aspects, the third, fourth, or fifth linker comprises glycine and serine amino acid residues. In some aspects, the linker comprises (GGGS)n, (SEQ ID NO:191) wherein n=1, 2, 3, 4, 5, 6, 7, or 8, or any range derivable therein, or GGGSGGGS (SEQ ID NO:47). In some aspects, the second linker comprises (GGGS)n (SEQ ID NO:191), wherein n=6. In some aspects, the polypeptide comprises a protein tag. In some aspects, the protein tag comprises a 6H tag (SEQ ID NO:87). In some aspects, the protein tag comprises a protein tag described herein. In some aspects, the polypeptide is not operatively linked to a particle, nanovesicle, or liposome. In some aspects, the composition does not comprise a liposome, particle, or nanovescicle.

The compositions may comprise or further comprise an additional polypeptide. In aspects of the disclosure, the methods may comprise or further comprise administration of an additional polypeptide. The additional polypeptide may be a polypeptide described herein. In some aspects, the composition comprises an additional polypeptide, wherein the polypeptide is an IL-12 p35 or p40 subunit. In some aspects, the additional polypeptide comprises a polypeptide of SEQ ID NO:3 or 4 or an amino acid sequence with at least 70% sequence identity to SEQ ID NO:3 or 4. In some aspects, the additional polypeptide comprises an amino acid sequence having or having at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% sequence identity to SEQ ID NO:3 or 4, or any derivable range therein.

In some aspects, the methods or the disclosure relate to the treatment of skin cancer, such as for the treatment of melanoma. In some aspects, the cancer comprises melanoma, colon, or breast cancer. In some aspects, the cancer comprises a solid tumor.

In some aspects, methods of the disclosure further comprise administration of one or more additional cancer therapies. In some aspects, the additional therapy is one described herein. In some aspects, the subject has or will receive an immunotherapy. In some aspects, the method further comprises administration of an immunotherapy. In a particular aspect, the immunotherapy comprises an immune checkpoint inhibitor. The immune checkpoint inhibitor may be an anti-PD-1 monoclonal antibody or an anti-CTLA-4 monoclonal antibody. Further exemplary immune checkpoint proteins that may be inhibited in aspects of the disclosure are described herein. In some aspects, the immune checkpoint inhibitor comprises one or more of nivolumab, pembrolizumab, pidilizumab, ipilimumab or tremelimumab. In some aspects, the immune checkpoint therapy is monotherapy. The term monotherapy, in the context of immune checkpoint therapy, refers to administration of one immune checkpoint inhibitor during the course of therapy. The monotherapy may be a therapy comprising of only one of a PD-1, PDL1, PDL2, CTLA-4, B7-1, or B7-2 inhibitor. In some aspects, the immune checkpoint inhibitor therapy comprises combination therapy. For example, the combination therapy may be a combination of (i) a PD-1, PDL1, or PDL2 inhibitor and (ii) a CTLA-4, B7-1, or B7-2 inhibitor. Particular combination therapies include those that comprise an anti-PD-1 antibody and an anti-CTLA-4 antibody. Further immunotherapies useful in the methods and compositions of the disclosure are described herein. In some aspects, the immunotherapy or additional therapy is administered before, after, or concurrent with the polypeptide. In some aspects, the polypeptide or composition is administered systemically. In some aspects, the polypeptide or composition is administered intratumorally. In some aspects, the polypeptide or composition is administered by a route of administration described herein. In some aspects, the polypeptide or composition is administered by intravenous injection. In some aspects, the subject has been previously treated with a cancer therapy. In some aspects, the subject has been determined to be non-responsive to the previous treatment or wherein the wherein the subject experienced non-specific toxicity to the previous treatment.

In some aspects, the toxicity of the treatment comprising the masked cytokine polypeptides of the disclosure is reduced compared to the toxicity of administration of the same dose of polypeptide that is unmasked. The reduction may be or may be at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any derivable range therein.

In some aspects, the serum IFN-gamma level induced by the masked cytokine polypeptide of the disclosure is significantly less than the serum IFN-gamma level induced by the unmasked cytokine polypeptide. In some aspects, the serum IFN-gamma level induced by the masked cytokine polypeptide of the disclosure is or is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any derivable range therein, less than the serum IFN-gamma level induced by the unmasked cytokine polypeptide.

In some aspects, the tumor IFN-gamma level induced by the masked cytokine polypeptide of the disclosure is significantly less than the tumor IFN-gamma level induced by the unmasked cytokine polypeptide. In some aspects, the tumor IFN-gamma level induced by the masked cytokine polypeptide of the disclosure is or is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any derivable range therein, less than the tumor IFN-gamma level induced by the unmasked cytokine polypeptide.

In some aspects, the tumor volume is significantly decreased by the masked cytokine polypeptide of the disclosure. In some aspects, the tumor volume is decreased by or decreased by at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any derivable range therein, after administration of the masked cytokine polypeptide of the disclosure. In some aspects, administration of the masked cytokine polypeptide cause a tumor volume decrease of or of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100%, or any derivable range therein, more than the decrease in tumor volume after administration of the unmasked cytokine polypeptide.

The term “cytokine polypeptide” as used herein refers to a polypeptide, which is cytokine or a receptor binding domain thereof and retains at a portion of cytokine activity. In certain aspects, the cytokine polypeptide is an active fragment of the cytokine.

The terms “protein”, “polypeptide” and “peptide” are used interchangeably herein when referring to a gene product comprising a polymer of amino acids.

The terms “subject,” “mammal,” and “patient” are used interchangeably. In some aspects, the subject is a mammal. In some aspects, the subject is a human. In some aspects, the subject is a mouse, rat, rabbit, dog, donkey, or a laboratory test animal such as fruit fly, zebrafish, etc.

It is contemplated that the methods and compositions include exclusion of any of the embodiments described herein.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other embodiments are discussed throughout this application. Any embodiment discussed with respect to one aspect of the disclosure applies to other aspects of the disclosure as well and vice versa.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Proposed mechanism of action of masked IL-12.

FIG. 2A-D. In vitro characterization of proIL12 constructs. a, Schematics illustrating designs of engineered proIL12 variants. b, Dose-response relationship of phosphorylated STAT4 with IL-12 and proIL12 variants in preactivated primary CD8⁺ T cells. c, Activation of MMP-sensitive (left) or uPA-sensitive (right) proIL12 molecules with corresponding proteases. d, In vitro cleavage of IL12Rβ1-VP-IL12 and of IL12Rβ1-LS-IL12 by MMPs and uPA. IL-12, IL12Rβ1-VP-IL12 and IL12Rβ1-LS-IL12 were either treated with assay buffer only (Blank), MMP2, MMP9 or uPA for 30 min at 37° C. Decrease of molecular weight from ˜105 kDa to ˜60 kDa indicates proteolytic cleavage of IL12Rβ1-VP-IL12 and IL12Rβ1-LS-IL12.

FIG. 3. Cleavage of different linkers by MMP2. ProIL12 molecules (all at the same concentration) containing indicated linker sequences were incubated with varying concentrations of MMP2 and cleavage was visualized via SDS-PAGE.

FIG. 4. Comparison of MMP2 sensitivity of L₄ and L₆.

FIG. 5. Body weight of C3H/HeJ mice. 8-week-old female C3H/HeJ mice were dosed s.c. daily for 8 days with indicated amounts of IL-12 or proIL12 (all on IL-12 basis, n=8/group). Mice were weighed the day after the last injection.

FIG. 6A-F. Quantification of circulating and intratumoral proinflammatory cytokines/chemokines. C57BL/6 mice were inoculated with B16F10 tumors on their back skin on day 0. On day 7, mice were treated i.v. with PBS, 5 mg IL-12, 15 mg M-L₅-IL12-CBD (on IL-12 basis) or 15 mg M-L₆-IL12-CBD (on IL-12 basis). On day 9, mice were bled, and sera were isolated. On day 10, mice were euthanized, and their tumors were harvested and homogenized. Serum IFNg (a), serum CCL4 (c), serum CCL2 (e), intratumoral IFNg (b), intratumoral CXCL9 (d), and intratumoral CCL4 (f) were measured via LEGENDPlex. Intratumoral cytokine/chemokine values were normalized by total protein concentration.

FIG. 7A-B. Antitumor Efficacy of proIL12 in syngeneic tumor models. a, C57BL/6 mice were inoculated with MC38 tumors on their back skin on day 0. On day 7, 10 and 13 mice were treated i.v. with PBS (n=7), 5 mg IL-12 (n=7), or 15 mg M-L₆-IL12-CBD (n=7; on IL-12 basis). b, C57BL/6 mice were inoculated with B16F10 tumors on their back skin on day 0. On day 7, 10 and 13 mice were treated i.v. with PBS (n=5), 15 mg M-L₆-IL12-CBD (n=8; on IL-12 basis), i.p. with 100 mg aPD-1 (n=5) or combination of M-L₆-IL12-CBD and aPD-1 (n=11).

FIG. 8. Activation of proIL12 by human tumor homogenate. IL-12 and proIL12 were diluted to 50 mg/mL and incubated overnight at 37° C. with 2 mg/mL of either human tumor lysate or ANT lysate. As negative control, proIL12 was also incubated with buffer only. Reaction mixtures were further serially diluted in medium and applied to preactivated mouse CD8⁺ T cells (n=2 per well) at indicated concentration.

FIG. 9. Non-cleavable masked IL-12 minimizes systemic IFNγ production while maintaining intratumoral IFNγ. B16F10 melanoma-bearing mice were treated once intravenously with 5 μg IL-12 or 15 μg M-(G₃S)₁₁-IL12. Plasma and tumors were collected 2 and 3 days after cytokine treatment, respectively. Plasma IFNγ (left) and intratumoral IFNγ (right, normalized by total protein) were quantified using LEGENDPlex.

FIG. 10. Non-cleavable masked IL-12 exerts antitumor efficacy in MC38 colon cancer. On day 0, mice were inoculated with MC38 colon cancer cells subcutaneously and on days 7, 10 and 13 were treated intravenously either with saline or 15 μg M-(G₃S)₁₁-IL12. Average tumor sizes (top) and individual tumor curves (bottom) are shown.

### DETAILED DESCRIPTION

Cytokines are key factors for antitumor activities, but not many of them have been translated to the clinic to date. IL12 is one of the strongest antitumor cytokines, but due to its high toxicity, the clinical trial has been terminated or unsuccessful. Thus, decreasing its toxicity is an important strategy to translate it to the clinic. To improve CBD-IL12 therapy, a domain of the IL12 receptor IL12Rβ1 was fused to the IL12, to form IL12Rβ1-IL12. This fusion is inactive, but the inclusion of an MMP or thrombin cleavage site between the receptor making agent and the cytokine yields a pro-cytokine that can be activated in the tumor microenvironment. The inventors have demonstrated that the immunotoxicity of the IL12 is thus reduced, and that the IL12Rβ1-IL12 fusion with the protease-sensitive linker retains therapeutic utility. The inventors also found that introducing multi-cleavage sites in the linker (e.g. tandem MMP, tandem thrombin, and MMP-thrombin dyads and repeats) would increase the protease sensitivity and may increase the antitumor efficacy of IL12Rβ1-IL12 therapy. Furthermore, the use of a collagen binding domain fused to the masked therapeutic molecules of the disclosure is particularly useful, since the CBD increases the retention of the masked therapeutic agent in the tumor microenvironment, which prolongs the exposure of the masked therapeutic agent to the protease and increases the local concentration of the unmasked therapeutic agent. In conclusion, the inventors have developed a technology to reduce toxicity of therapeutic agents by fusing the cytokine receptor to the cytokine. Tumor specific proteases cleave the linker to activate the cytokine within the tumor.

### I. POLYPEPTIDE ASPECTS

### A. Therapeutic Agents and Masking Agents

Aspects of the disclosure relate to therapeutic agent and masking agents that bind to the therapeutic agent and prevent association of the therapeutic agent with its target to reduce toxicity associated with the therapeutic agent. The polypeptides of the disclosure comprise a tumor-associated protease cleavage site that unmasks the therapeutic agent when it encounters the relevant protease. Since the protease is one that is enriched in the tumor microenvironment, there is a reduction of the active therapeutic agent in normal tissues when administered systemically, compared to the systemic administration of the unmasked therapeutic agent.

### 1. Cytokines and Masking Polypeptides

In some aspects, the therapeutic agent comprises a cytokine or a therapeutic polypeptide from a cytokine. In certain aspects, the cytokine comprises a functionally active fragment of a cytokine. In some aspects, the functionally active cytokine fragment binds and activates the corresponding receptor. In some aspects, the cytokine comprises IL12. IL12 is a heterodimeric glycosylated cytokine comprised of disulfide-linked p35 (˜35 kDa) and p40 (˜40 kDa) subunits. The human IL12 p35 sequence is represented by the following: RNLPVATPDPGMFPCLHHSQNLLRAVSNMLQKARQTLEFYPCTSEEIDHEDITKDKTST VEACLPLELTKNESCLNSRETSFITNGSCLASRKTSFMMALCLSSIYEDLKMYQVEFKTM NAKLLMDPKRQIFLDQNMLAVIDELMQALNFNSETVPQKSSLEEPDFYKTKIKLCILLHA FRIRAVTIDRVMSYLNAS (SEQ ID NO:3). The human IL12 p40 sequence is represented by the following: IWELKKDVYVVELDWYPDAPGEMVVLTCDTPEEDGITWTLDQSSEVLGSGKTLTIQVK EFGDAGQYTCHKGGEVLSHSLLLLHKKEDGIWSTDILKDQKEPKNKTFLRCEAKNYSGR FTCWWLTTISTDLTFSVKSSRGSSDPQGVTCGAATLSAERVRGDNKEYEYSVECQEDSA CPAAEESLPIEVMVDAVHKLKYENYTSSFFIRDIIKPDPPKNLQLKPLKNSRQVEVSWEY PDTWSTPHSYFSLTFCVQVQGKSKREKKDRVFTDKTSATVICRKNASISVRAQDRYYSS SWSEWASVPCS (SEQ ID NO:4). The mouse IL12 p35 sequence is represented by the following: RVIPVSGPARCLSQSRNLLKTTDDMVKTAREKLKHYSCTAEDIDHEDITRDQTSTLKTCL PLELHKNESCLATRETSSTTRGSCLPPQKTSLMMTLCLGSIYEDLKMYQTEFQAINAALQ NHNHQQIILDKGMLVAIDELMQSLNHNGETLRQKPPVGEADPYRVKMKLCILLHAFSTR VVTINRVMGYLSSA (SEQ ID NO:5). The mouse IL12 p40 sequence is represented by the following:

(SEQ ID NO: 6)
MWELEKDVYVVEVDWTPDAPGETVNLTCDTPEEDDITWTSDQRHGVIGSG
KTLTITVKEFLDAGQYTCHKGGETLSHSHLLLHKKENGIWSTEILKNFKN
KTFLKCEAPNYSGRFTCSWLVQRNMDLKFNIKSSSSSPDSRAVTCGMASL
SAEKVTLDQRDYEKYSVSCQEDVTCPTAEETLPIELALEARQQNKYENYS
TSFFIRDIIKPDPPKNLQMKPLKNSQVEVSWEYPDSWSTPHSYFSLKFFV
RIQRKKEKMKETEEGCNQKGAFLVEKTSTEVQCKGGNVCVQAQDRYYNSS
CSKWACVPCRVRS.

Suitable IL12 masking agents include polypeptides that bind to IL12 and prevent binding of IL12 to other molecules, such as IL12R. Exemplary polypeptides include polypeptides from IL12R, such as IL12Rβ1 and IL12Rβ2.

The mouse IL12Rβ1 is represented by a polypeptide with either of the following amino acid sequences:

(SEQ ID NO: 2)
QLGASGPGDGCCVEKTSFPEGASGSPLGPRNLSCYRVSKTDYECSWQYDG
PEDNVSHVLWCCFVPPNHTHTGQERCRYFSSGPDRTVQFWEQDGIPVLSK
VNFWVESRLGNRTMKSQKISQYLYNWTKTTPPLGHIKVSQSHRQLRMDWN
VSEEAGAEVQFRRRMPTTNWTLGDCGPQVNSGSGVLGDIRGSMSESCLCP
SENMAQEIQIRRRRRLSSGAPGGPWSDWSMPVCVPPEVLPQALVPRGS
and
(SEQ ID NO: 20)
QLGASGPGDGCCVEKTSFPEGASGSPLGPRNLSCYRVSKTDYECSWQYDG
PEDNVSHVLWCCFVPPNHTHTGQERCRYFSSGPDRTVQFWEQDGIPVLSK
VNFWVESRLGNRTMKSQKISQYLYNWTKTTPPLGHIKVSQSHRQLRMDWN
VSEEAGAEVQFRRRMPTTNWTLGDCGPQVNSGSGVLGDIRGSMSESCLCP
SENMAQEIQIRRRRRLSSGAPGGPWSDWSMPVCVPPEVLPQAKIKFLVEP
LNQGGRRRLTMQGQSPQLAVPEGCRGRPGAQVKKHLVLVRMLSCRCQAQT
SKTVPLGKKLNLSGATYDLNVLAKTRFGRSTIQKWHLPAQELTETRALNV
SVGGNMTSMQWAAQAPGTTYCLEWQPWFQHRNHTHCTLIVPEEEDPAKMV
THSWSSKPTLEQEECYRITVFASKNPKNPMLWATVLSSYYFGGNASRAGT
PRHVSVRNQTGDSVSVEWTASQLSTCPGVLTQYVVRCEAEDGAWESEWLV
PPTKTQVTLDGLRSRVMYKVQVRADTARLPGAWSHPQRFSFEVQISRLSI
IFASLGSFASVLLVGSLGYIGLNRAAWHLCPPLPTPCGSTAVEFPGSQGK
QAWQWCNPEDFPEVLYPRDALVVEMPGDRGDGTESPQAAPECALDTRRPL
ETQRQRQVQALSEARRLGLAREDCPRGDLAHVTLPLLLGGVTQGASVLDD
LWRTHKTAEPGPPTLGQEA.

The human IL12Rβ1 is represented by a polypeptide with the following amino acid sequence:

(SEQ ID NO: 19)
CRTSECCFQDPPYPDADSGSASGPRDLRCYRISSDRYECSWQYEGPTAGV
SHFLRCCLSSGRCCYFAAGSATRLQFSDQAGVSVLYTVTLWVESWARNQT
EKSPEVTLQLYNSVKYEPPLGDIKVSKLAGQLRMEWETPDNQVGAEVQFR
HRTPSSPWKLGDCGPQDDDTESCLCPLEMNVAQEFQLRRRQLGSQGSSWS
KWSSPVCVPPENPPQPQVRFSVEQLGQDGRRRLTLKEQPTQLELPEGCQG
LAPGTEVTYRLQLHMLSCPCKAKATRTLHLGKMPYLSGAAYNVAVISSNQ
FGPGLNQTWHIPADTHTEPVALNISVGTNGTTMYWPARAQSMTYCIEWQP
VGQDGGLATCSLTAPQDPDPAGMATYSWSRESGAMGQEKCYYITIFASAH
PEKLTLWSTVLSTYHFGGNASAAGTPHHVSVKNHSLDSVSVDWAPSLLST
CPGVLKEYVVRCRDEDSKQVSEHPVQPTETQVTLSGLRAGVAYTVQVRAD
TAWLRGVWSQPQRFSIEVQVSDWLIFFASLGSFLSILLVGVLGYLGLNRA
ARHLCPPLPTPCASSAIEFPGGKETWQWINPVDFQEEASLQEALVVEMSW
DKGERTEPLEKTELPEGAPELALDTELSLEDGDRCKAKM.

The human IL12Rβ2 is represented by a polypeptide with the following amino acid sequence: KIDACKRGDVTVKPSHVILLGSTVNITCSLKPRQGCFHYSRENKLILYKFDRRINFHHGH SLNSQVTGLPLGTTLFVCKLACINSDEIQICGAEIFVGVAPEQPQNLSCIQKGEQGTVACT WERGRDTHLYTEYTLQLSGPKNLTWQKQCKDIYCDYLDFGINLTPESPESNFTAKVTAV NSLGSSSSLPSTFTFLDIVRPLPPWDIRIKFQKASVSRCTLYWRDEGL VLLNRLRYRPSNS RLWNMVNVTKAKGRHDLLDLKPFTEYEFQISSKLHLYKGSWSDWSESLRAQTPEEEPT GMLDVWYMKRHIDYSRQQISLFWKNLSVSEARGKILHYQVTLQELTGGKAMTQNITGH TSWTTVIPRTGNWAVAVSAANSKGSSLPTRINIMNLCEAGLLAPRQVSANSEGMDNILV TWQPPRKDPSAVQEYVVEWRELHPGGDTQVPLNWLRSRPYNVSALISENIKSYICYEIR VYALSGDQGGCSSILGNSKHKAPLSGPHINAITEEKGSILISWNSIPVQEQMGCLLHYRIY WKERDSNSQPQLCEIPYRVSQNSHPINSLQPRVTYVLWMTALTAAGESSHGNEREFCLQ GKANWMAFVAPSICIAIIMVGIFSTHYFQQKVFVLLAALRPQWCSREIPDPANSTCAKKY PIAEEKTQLPLDRLLIDWPTPEDPEPLVISEVLHQVTPVFRHPPCSNWPQREKGIQGHQAS EKDMMHSASSPPPPRALQAESRQLVDLYK VLESRGSDPKPENPACPWTVLPAGDLPTHD GYLPSNIDDLPSHEAPLADSLEELEPQHISLSVFPSSSLHPLTFSCGDKLTLDQLKMRCDS LML (SEQ ID NO:21). The mouse IL12Rβ2 is represented by a polypeptide with the following amino acid sequence:

(SEQ ID NO: 22)
NIDVCKLGTVTVQPAPVIPLGSAANISCSLNPKQGCSHYPSSNELILLKF
VNDVLVENLHGKKVHDHTGHSSTFQVTNLSLGMTLFVCKLNCSNSQKKPP
VPVCGVEISVGVAPEPPQNISCVQEGENGTVACSWNSGKVTYLKTNYTLQ
LSGPNNLTCQKQCFSDNRQNCNRLDLGINLSPDLAESRFIVRVTAINDLG
NSSSLPHTFTFLDIVIPLPPWDIRINFLNASGSRGTLQWEDEGQVVLNQL
RYQPLNSTSWNMVNATNAKGKYDLRDLRPFTEYEFQISSKLHLSGGSWSN
WSESLRTRTPEEEPVGILDIWYMKQDIDYDRQQISLFWKSLNPSEARGKI
LHYQVTLQEVTKKTTLQNTTRHTSWTRVIPRTGAWTASVSAANSKGASAP
THINIVDLCGTGLLAPHQVSAKSENMDNILVTWQPPKKADSAVREYIVEW
RALQPGSITKFPPHWLRIPPDNMSALISENIKPYICYEIRVHALSESQGG
CSSIRGDSKHKAPVSGPHITAITEKKERLFISWTHIPFPEQRGCILHYRI
YWKERDSTAQPELCEIQYRRSQNSHPISSLQPRVTYVLWMTAVTAAGESP
QGNEREFCPQGKANWKAFVISSICIAIITVGTFSIRYFRQKAFTLLSTLK
PQWYSRTIPDPANSTWVKKYPILEEKIQLPTDNLLMAWPTPEEPEPLIIH
EVLYHMIPVVRQPYYFKRGQGFQGYSTSKQDAMYIANPQATGTLTAETRQ
LVNLYKVLESRDPDSKLANLTSPLTVTPVNYLPSHEGYLPSNIEDLSPHE
ADPTDSFDLEHQHISLSIFASSSLRPLIFGGERLTLDRLKMGYDSLMSNE
A.

In some aspects, the cytokine comprises a polypeptide comprising an amino acid sequence of SEQ ID NO:3-6, or a polypeptide comprising an amino acid sequence of a fragment of the polypeptides represented by SEQ ID NO:3-6, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:3-6 or a fragment thereof.

In some aspects, the cytokine comprises an IL12 polypeptide and the masking agent comprises an amino acid sequence of SEQ ID NO:2, or 19-22, or a polypeptide comprising an amino acid sequence of a fragment of the polypeptides represented by SEQ ID NO:2, or 19-22, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:2, or 19-22 or a fragment thereof.

In some aspects, the cytokine comprises IL-2. The human IL-2 sequence comprises MAPTSSSTKKTQLQLEHLLLDLQMILNGINNYKNPKLTRMLTFKFYMPKKATELKHLQC LEEELKPLEEVLNLAQSKNFHLRPRDLISNINVIVLELKGSETTFMCEYADETATIVEFLN RWITFAQSIISTLT (SEQ ID NO:23). The mouse IL-2 sequence comprises PTSSSTSSSTAEAQQQQQQQQQQQQHLEQLLMDLQELLSRMENYRNLKLPRMLTFKFY LPKQATELKDLQCLEDELGPLRHVLDLTQSKSFQLEDAENFISNIRVTVVKLKGSDNTFE CQFDDESATVVDFLRRWIAFCQSIISTSPQ (SEQ ID NO:24).

In some aspects, the masking agent for IL-2 comprises an IL-2R polypeptide. In some aspects, the IL-2R polypeptide comprises a polypeptide from the IL-2Rbeta, IL-2Ralpha, or IL-2Rgamma subunit. Human Interleukin-2 receptor subunit beta comprises the following amino acid sequence: AVNGTSQFTCFYNSRANISCVWSQDGALQDTSCQVHAWPDRRRWNQTCELLPVSQAS WACNLILGAPDSQKLTTVDIVTLRVLCREGVRWRVMAIQDFKPFENLRLMAPISLQVVH VETHRCNISWEISQASHYFERHLEFEARTLSPGHTWEEAPLLTLKQKQEWICLETLTPDT QYEFQVRVKPLQGEFTTWSPWSQPLAFRTKPAALGKDT (SEQ ID NO:27). Mouse Interleukin-2 receptor subunit beta has the following amino acid sequence: AVKNCSHLECFYNSRANVSCMWSHEEALNVTTCHVHAKSNLRHWNKTCELTLVRQAS WACNLILGSFPESQSLTSVDLLDINVVCWEEKGWRRVKTCDFHPFDNLRLVAPHSLQVL HIDTQRCNISWKVSQVSHYIEPYLEFEARRRLLGHISWEDASVLSLKQRQQWLFLEMLIPS TSYEVQVRVKAQRNNTGTWSPWSQPLTFRTRPADPMKE (SEQ ID NO:28). Human Interleukin-2 receptor subunit alpha has the following amino acid sequence: ELCDDDPPEIPHATFKAMAYKEGTMLNCECKRGFRRIKSGSLYMLCTGNSSHSSWDNQ CQCTSSATRNTTKQVTPQPEEQKERKTTEMQSPMQPVDQASLPGHCREPPPWENEATER IYHFVVGQMVYYQCVQGYRALHRGPAESVCKMTHGKTRWTQPQLICTGEMETSQFPG EEKPQASPEGRPESETSCLVTTTDFQIQTEMAATMETSIFTTEYQ (SEQ ID NO:29). Mouse Interleukin-2 receptor subunit alpha has the following amino acid sequence: ELCLYDPPEVPNATFKALSYKNGTILNCECKRGFRRLKEL VYMRCLGNSWSSNCQCTSN SHDKSRKQVTAQLEHQKEQQTTTDMQKPTQSMHQENLTGHCREPPPWKHEDSKRIYHF VEGQSVHYECIPGYKALQRGPAISICKMKCGKTGWTQPQLTCVDEREHHRFLASEESQG SRNSSPESETSCPITTTDFPQPTETTAMTETFVLTMEYK (SEQ ID NO:30). Human Interleukin-2 receptor subunit gamma has the following amino acid sequence: LNTTILTPNGNEDTTADFFLTTMPTDSLSVSTLPLPEVQCFVFNVEYMNCTWNSSSEPQP TNLTLHYWYKNSDNDKVQKCSHYLFSEEITSGCQLQKKEIHLYQTFVVQLQDPREPRRQ ATQMLKLQNL VIPWAPENLTLHKLSESQLELNWNNRFLNHCLEHLVQYRTDWDHSWT EQSVDYRHKFSLPSVDGQKRYTFRVRSRFNPLCGSAQHWSEWSHPIHWGSNTSKENPFL FALEA (SEQ ID NO:31). Mouse Interleukin-2 receptor subunit gamma has the following amino acid sequence:

(SEQ ID NO: 32)
WSSKVLMSSANEDIKADLILTSTAPEHLSAPTLPLPEVQCFVFNIEYMNC
TWNSSSEPQATNLTLHYRYKVSDNNTFQECSHYLFSKEITSGCQIQKEDI
QLYQTFVVQLQDPQKPQRRAVQKLNLQNLVIPRAPENLTLSNLSESQLEL
RWKSRHIKERCLQYLVQYRSNRDRSWTELIVNHEPRFSLPSVDELKRYTF
RVRSRYNPICGSSQQWSKWSQPVHWGSHTVEENPSLFALEA.

In some aspects, the cytokine comprises a polypeptide comprising an amino acid sequence of SEQ ID NO:23 or 24, or a polypeptide comprising an amino acid sequence of a fragment of the polypeptides represented by SEQ ID NO:23 and 24, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:23 or 24 or a fragment thereof.

In some aspects, the cytokine comprises an IL-2 polypeptide and the masking agent comprises an amino acid sequence of SEQ ID NO:27-32, or a polypeptide comprising an amino acid sequence of a fragment of the polypeptides represented by SEQ ID NO:27-32, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:27-32 or a fragment thereof.

In some aspects, the cytokine comprises IFNγ. The mouse IFNγ comprises the following sequence: HGTVIESLESLNNYFNSSGIDVEEKSLFLDIWRNWQKDGDMKILQSQIISFYLRLFEVLKD NQAISNNISVIESHLITTFFSNSKAKKDAFMSIAKFEVNNPQVQRQAFNELIRVVHQLLPE SSLRKRKRSRC (SEQ ID NO:25). The human IFNγ comprises the following sequence: QDPYVKEAENLKKYFNAGHSDVADNGTLFLGILKNWKEESDRKIMQSQIVSFYFKLFK NFKDDQSIQKSVETIKEDMNVKFFNSNKKKRDDFEKLTNYSVTDLNVQRKAIHELIQVM AELSPAAKTGKRKRSQMLFQGRRASQ (SEQ ID NO:26). It is also contemplated that IFNg of SEQ ID NO:25 and 26 comprise a methionine as the first amino acid. The IFNγ may be a functional fragment, such as one that is truncated at the C-terminus. For example, the IFNγ polypeptide may be one comprising at least 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 contiguous amino acids of SEQ ID NO:25 or 26. In some aspects, the IFNγ polypeptide may be one comprising at least amino acids 1 to 30, 31, 32, 33, 34, 35, 36, 37 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, or 132 of SEQ ID NO:25 or 26 (or any range derivable therein).

In some aspects, the masking agent for a IFNγ polypeptide comprises a polypeptide from the IFNγ receptor 1 or IFNγ receptor 2. The human IFNγ receptor 1 comprises the following sequence:

(SEQ ID NO: 33)
EMGTADLGPSSVPTPTNVTIESYNMNPIVYWEYQIMPQVPVFTVEVKNY
GVKNSEWIDACINISHHYCNISDHVGDPSNSLWVRVKARVGQKESAYAK
SEEFAVCRDGKIGPPKLDIRKEEKQIMIDIFHPSVFVNGDEQEVDYDPE
TTCYIRVYNVYVRMNGSEIQYKILTQKEDDCDEIQCQLAIPVSSLNSQY
CVSAEGVLHVWGVTTEKSKEVCITIFNSSIKG.

The mouse IFNγ receptor 1 comprises the following sequence: ALTSTEDPEPPSVPVPTNVLIKSYNLNPVVCWEYQNMSQTPIFTVQVKVYSGSWTDSCT NISDHCCNIYEQIMYPDVSAWARVKAKVGQKESDYARSKEFLMCLKGKVGPPGLEIRR KKEEQLSVLVFHPEVVVNGESQGTMFGDGSTCYTFDYTVYVEHNRSGEILHTKHTVEK EECNETLCELNISVSTLDSRYCISVDGISSFWQVRTEKSKDVCIPPFHDDRKDS (SEQ ID NO:34). The human IFNγ receptor 2 comprises the following sequence: SQLPAPQHPKIRLYNAEQVLSWEPVALSNSTRPVVYQVQFKYTDSKWFTADIMSIGVNC TQITATECDFTAASPSAGFPMDFNVTLRLRAELGALHSAWVTMPWFQHYRNVTVGPPE NIEVTPGEGSLIIRFSSPFDIADTSTAFFCYYVHYWEKGGIQQVKGPFRSNSISLDNLKPSR VYCLQVQAQLLWNKSNIFRVGHLSNISCYETMADASTELQQ (SEQ ID NO:35). The mouse IFNγ 2 comprises the following sequence: receptor

(SEQ ID NO: 36)
ASSPDSFSQLAAPLNPRLHLYNDEQILTWEPSPSSNDPRPVVYQVEYSF
IDGSWHRLLEPNCTDITETKCDLTGGGRLKLFPHPFTVFLRVRAKRGNL
TSKWVGLEPFQHYENVTVGPPKNISVTPGKGSLVIHFSPPFDVFHGATF
QYLVHYWEKSETQQEQVEGPFKSNSIVLGNLKPYRVYCLQTEAQLILKN
KKIRPHGLLSNVSCHETTANASARLQQVILIPLGIFALLLGLTGACFTL
FLKYQSRVKYWFQAPPNIPEQIEEYLKDPDQFILEVLDKDGSPKEDSWD
SVSIISSPEKERDDVLQTP.

In some aspects, the cytokine comprises a polypeptide comprising an amino acid sequence of SEQ ID NO:25 or 26, or a polypeptide comprising an amino acid sequence of a fragment of the polypeptides represented by SEQ ID NO:25 and 26, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:25 or 26 or a fragment thereof.

In some aspects, the cytokine comprises an IFNγ polypeptide and the masking agent comprises an amino acid sequence of SEQ ID NO:33-36, or a polypeptide comprising an amino acid sequence of a fragment of the polypeptides represented by SEQ ID NO:33-36, or a polypeptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of SEQ ID NO:33-36 or a fragment thereof.

In aspects of the disclosure, the masking agent made be a polypeptide or a functional fragment of a polypeptide described herein. In some aspects, the masking agent comprises a receptor polypeptide or a fragment thereof that binds to the cytokine.

### 2. Antibodies

In some aspects, the therapeutic agent comprises an antibody, such as a therapeutic antibody. In some aspects, the therapeutic antibody is modified by the site-specific substitution of an amino acid in either the heavy or light chain variable region with a cysteine (Cys). The sulfhydryl (SH) group in the side chain of the substituted-in Cys serves as a chemical handle for attaching a masking agent that interferes with the antibody's ability to bind to its antigen. The masking agent may be a group that sterically inhibits antibody-antigen binding, but otherwise does not specifically interact with either the antibody or the antigen.

Alternatively, the masking agent can interact with the antibody, for example by electrostatic or van der Waals forces. A tumor-associated protease cleavage site may be between or link the masking agent and the antibody.

In some aspects, the masking agent can have pharmacological activity of its own after its release by cleavage of the tumor-associated protease cleavage site. The Cys substitution sites are selected such that replacing the original amino acid with a Cys does not detrimentally affect the ability of the antibody to specifically and strongly bind to its antigen. Further, removal of the masking agent can leave behind a residual chemical group still covalently attached to the Cys. In one aspect, there is provided a prodrugged antibody according to formula (I):

(M-L)ₘ-Ab I

wherein Ab is an antibody having at least one amino acid in its heavy or light chain variable region replaced by a Cys, wherein the replaced amino acid (a) is in a framework region; (b) has a side chain exposure of at least 30% and (c) is within 10 A, preferably 5 A, of a CDR amino acid; M is a masking agent that inhibits binding of Ab to its antigen; each L is, independently, a linker moiety bonded to M and Ab, L comprising a tumor-associated cleavage site and being bonded to Ab at aforesaid Cys; and m is 1, 2, 3, or 4.

In some aspects, the at least one replaced amino in antibody Ab is at Kabat position 1, 3, 5, 19, 23, 25, 43, 46, 68, 72, 74, 75, 76, 82a, 82b, 83, 84, 85, or 105 of the heavy chain variable region or at Kabat position 1, 3, 5, 7, 8, 18, 20, 45, 57, 60, 63, 65, 66, 67, 69, 77, or 100 of the light chain variable region. In some aspects, the at least one replaced amino acid in antibody Ab is at Kabat position 23 of the heavy chain or Kabat position 67 of the light chain. In some aspects, there is provided an antibody having a Cys at Kabat position 67 of the light chain. The antibody can be an anti-CTLA4 antibody or an anti-CD137 antibody. In some aspects, there is provided an antibody having a Cys at Kabat position 23 of the heavy chain. The antibody can be an anti-CTLA4 antibody or an anti-CD137 antibody.

The masked therapeutic antibody of the disclosure can be polyclonal, monoclonal, mouse, human, humanized, or chimeric. Suitable amino acids in the heavy and light chain variable regions for substitution with a Cys are framework amino acids whose side chains are solvent exposed—preferably at least 30% exposed—so that the substituted-in Cys is accessible for attachment of the masking agent. It is also important that the substituted-out amino acid is near a CDR amino acid, so that the masking agent can effectively interfere with antibody-antigen binding. A distance of no more than 10 A is preferred, more preferably no more than 5 A. Preferred positions for Cys substitution include positions 23 in the heavy chain variable region and 67 in the light chain variable region, numbering per Kabat. Both positions are in the framework region of the respective variable regions. A Cys can be substituted into these positions by site-specific substitution techniques well known in the art. A substitution at the first site can be referred to, using a shorthand notation, as VL X67C, where X denotes the substituted-out amino acid. In native antibodies, this site is highly conserved and is often Ser. A substitution at the second site can be similarly referred to as VH X23C.

A masked antibody of this disclosure can have either a substitution in the VH region or in the VL region, or both. If the antibody has only one of these substitutions, the theoretical maximum number of blocking moiety-linker compounds that can be attached is two, although a masked antibody preparation may assay statistically for a lower number, reflecting chemical inefficiency in the attachment process. If the antibody has both substitutions, the theoretical maximum number is four.

In some aspects, the antibody is a bispecific antibody, which has two different pairs of heavy and light chains. Thus, a masked antibody of this disclosure can be a bispecific antibody in which only one heavy/light chain pair has been masked or one in which both heavy/light chain pairs have been masked. The substitution of an amino acid in a VH or VL region with a Cys, for the purpose of introducing a sulfhydryl side chain amenable to conjugation by maleimide addition chemistry to make an antibody-drug conjugate, is also known. See, for example, Eigenbrot et al. 2007 and Bhakta et a/. 2016).

Masking agents that can be used to interfere with or block activity of a masked antibody with its antigen include: polyethylene glycol (PEG), an albumin binding polypeptide, adnectin, a peptide, and a soluble globular protein such as albumin or fibrinogen. In some aspects, the blocking agent comprises PEG having a molecular weight of at least about 2 kDa, with 2 kDa corresponding to PEG with about 45 —(CH2CH2O)— repeating units, and preferably PEG with a molecular weight of at least about 5 kDa, with 5 kDa corresponding to PEG with about 115 —(CH2CH2O)— repeating units.

An antibody having a Cys as described herein can be conjugated to a masking agent having a maleimide terminal group by Michael addition of the Cys sulfhydryl (SH), as known in the art. The procedures for such conjugation are well known in the art; see, for example, Shepard et al, WO 2017/112624 A 1 (2017), which is herein incorporated by reference. Further examples of specific masking agents for therapeutic antibodies are disclose in WO2019036433, which is herein incorporated by reference.

In further aspects, the therapeutic agent may be an antibody in which variable regions are masked by linkage of the N-termini of variable regions chains to coiled-coil forming peptides. The coiled-coil forming peptides associate with one another to form coiled coils (i.e., the respective peptides each form coils and these coils are coiled around each other). The coiled coils may sterically inhibit binding of the antibody binding site to its target. In some aspects, the antibody comprises a bivalent antibody. Non-covalent associations between the coiled coil forming peptides are sufficient to form a stable coiled coils inhibiting binding of the antibody variable region; it is not for example necessary for the coiled-coil forming peptides to be further linked by a disulfide bridge between terminal cysteines of the respective peptides. The presence of non-naturally occurring cysteines is potentially disadvantageous because they can lead to misfolding or misconjugation problems. Masking of antibodies by this format can reduce binding affinities (and cytotoxic activities in the case of ADC's) by over a hundred fold. Antibodies can be masked in this format without significant impairment of expression, purification, conjugation, pharmacokinetics, or binding or other activity on unmasking.

In some aspects, the masking agent comprises a coiled coil. Coiled coil forming peptides are peptide pairs that can associate with one another to form coiled coils. “Coiled coils” is a term of art referring to bundles of alpha-helices wound into superhelical structures. Leucine zipper forming peptides are one example of peptides associating to form coiled coils. The coiled coils formed in the current disclosure typically are formed from two coiled coil forming peptides. Coiled coils can form with alpha helices on the peptides in parallel or opposite orientations. Coiled coils are further characterized by packing of amino acid side chains in the core of the bundle, called knobs-into-holes, in which a residue from one helix (knob) packs into a space surrounded by four side chains of the facing helix (hole). The residues engaged in knobs-into-holes interactions are usually hydrophobic, whereas the outer residues are hydrophilic, the sequence of coiled coils therefore shows a ‘heptad’ repeat in the chemical nature of side chains. Examples of consensus formulae for heptad repeats in coiled coils forming peptides are provided by WO2011034605, which is herein incorporated by reference.

In some aspects, the coiled coil comprises Formula II:

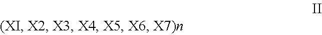

wherein XI is a hydrophobic amino acid or asparagine; X2, X3 and X6 are any amino acid; X4 is a hydrophobic amino acid; and X5 and X7 are each a charged amino acid residue.

Examples of coiled coil comprise:

(SEQ ID NO: 58)
GASTSVDELQAEVDQLQDENYALKTKVAQLRKKVEKLSE;
(SEQ ID NO: 59)
GASTTVAQLRERVKTLRAQNYELESEVQRLREQVAQLA;
(SEQ ID NO: 60)
EACGASTSVDELQAEVDQLQDENYALKTKVAQLRKKVEKLSE;
(SEQ ID NO: 61)
EACGASTTVAQLRERVKTLRAQNYELESEVQRLREQVAQLA;
(SEQ ID NO: 62)
LEIEAAFLERENTALETRVAELRQRVQRARNRVSQYRTRY;
(SEQ ID NO: 63)
LEIRAAFLRQRNTALRTEVAELEQEVQRLENEVSQYETRY;
(SEQ ID NO: 64)
EACGALEIEAAFLERENTALETRVAELRQRVQRARNRVSQYRTRY;
(SEQ ID NO: 65)
EACGALEIRAAFLRQRNTALRTEVAELEQEVQRLENEVSQYETRY;
(SEQ ID NO: 66)
LEIRAAFLRRRNTALRTRVAELRQRVQRLRNIVSQYETRY;
(SEQ ID NO: 67)
LEIEAAFLEQENTALETEVAELEQEVQRLENIVSQYETRY;
(SEQ ID NO: 68)
EACGALEIRAAFLRRRNTALRTRVAELRQRVQRLRNIVSQYETRY;
(SEQ ID NO: 69)
EACGALEIEAAFLEQENTALETEVAELEQEVQRLENIVSQYETRY;
(SEQ ID NO: 70)
QGASTSVDELQAEVDQLEDENYALKTKVAQLRKKVEKL;
(SEQ ID NO: 71)
QGASTTVAQLEEKVKTLRAENYELKSEVQRLEEQVAQL;
(SEQ ID NO: 72)
EACGASTSVDELQAEVDQLEDENYALKTKVAQLRKKVEKL;
(SEQ ID NO: 73)
EACGASTTVAQLEEKVKTLRAENYELKSEVQRLEEQVAQL;
(SEQ ID NO: 74)
AGLTDTLQAETDQLEDKKSALQTEIANLLKEKEKLEFILAAH;
(SEQ ID NO: 75)
AGRIARLEEKVKTLKAQNSELASTANMLREQVAQLKQKVMNY;
(SEQ ID NO: 76)
EACGAGLTDTLQAETDQLEDKKSALQTEIANLLKEKEKLEFILAAH;
(SEQ ID NO: 77)
EACGAGRIARLEEKVKTLKAQNSELASTANMLREQVAQLKQKVMNY;
(SEQ IDNO: 78)
GKIAALKQKIAALKYKNAALKKKIAALKQ;
(SEQ ID NO: 79)
GEIAALEQEIAALEKENAALEWEIAALEQ;
(SEQ ID NO: 80)
EACGAGKIAALKOKIAALKYKNAALKKKIAALKQ;
and
(SEQ ID NO: 81)
EACGAGEIAALEQEIAALEKENAALEWEIAALEQ.

Coiled coils forming peptides are linked to the N-termini of antibody variable regions via a linker including a tumor-associated protease cleavage site. A typical antibody includes a heavy and light chain variable region, in which case a coiled-coil forming peptide is linked to the N-termini of each. A bivalent antibody has two binding sites, which may or may not be the same. In a normal monospecific antibody, the binding sites are the same and the antibody has two identical light and heavy chain pairs. In this case, each heavy chain is linked to the same coiled-coil forming peptide and each light chain to the same coiled-coil forming peptide (which may or may not be the same as the peptide linked to the heavy chain).

In a bispecific antibody, the binding sites are different and formed from two different heavy and light chain pairs. The binding sites can have specificity for different targets or different epitopes on the same target. If the binding sites have specificity for different targets, the targets can be on the same cell (e.g., two different surface antigens on a cancer cell) or two different cells (e.g., one surface antigen on a cancer cell and one on an immune cell such as a T-cell). For example, one binding site of a bispecific antibody can be directed against CD3 or 4-1BB.

In a bispecific antibody, the heavy and light chain variable region of one binding site can be respectively linked to coiled-coil forming peptides. The heavy and light chain variable regions of the other binding site may or may not be also linked to coiled coil peptides. If the heavy light pairs of both binding sites are both linked to coiled coil peptides, then typically both heavy chain variable regions are linked to the same type of coiled-coil forming peptide as are both light chain variable regions. Masking of both binding sites can be useful, for example, if both binding sites have specificity for surface antigens on the same tumor. Masking of one but not both binding sites can be useful for example, when one binding site is specific for a tumor surface antigen and the other has specificity for a surface antigen on an immune cell. Either the binding site with specificity for the tumor surface antigen or for the immune cell antigen can be masked. Some bispecific antibodies with specificities to both a tumor surface antigen and an immune cell have masking of both sites.

Coiled coils can be formed from the same peptide forming a homodimer or two different peptides forming a heterodimer. For formation of a homodimer, light and heavy antibody chains are linked to the same coiled coil forming peptide. For formation of a heterodimer, light and heavy antibody chains are linked to different coiled coils peptides. For some pairs of coiled coil forming peptides, it is preferred that one of the pair be linked to the heavy chain and the other to the light chain of an antibody although the reverse orientation is also possible.

Each antibody chain can be linked to a single coiled coil forming peptide or multiple such peptides in tandem (e.g., two, three, four or five copies of a peptide). If the latter, the peptides in tandem linkage are usually the same. Also if tandem linkage is employed, light and heavy chains are usually linked to the same number of peptides.

Linkage of antibody chains to coiled coil forming peptides can reduce the binding affinity of an antibody by, for example, at least 10, 50, 100, 200, 500, 1000, 1500, 2000, 4000, 5000 or 10,000-fold relative to the same antibody without such linkage or after cleavage of such linkage. In some such antibodies, binding affinity is reduced 50-10,000, 50-5000, 50-4000, 50-1000, 100-10,000, 100-5000, 100-4000, 200-10,000, 200-5000, 50-1500, 100-1500, 200-1500, 200-1000, 500-1500, 50-1000, 100-1000, 200-1000, 500-1000, 50-500, 100-500 fold.

Antibodies include non-human, humanized, human, chimeric, and veneered antibodies, nanobodies, dAbs, scFV's, Fabs, and the like. Some such antibodies include immuno specific for a cancer cell antigen, preferably one on the cell surface internalizable within a cell on antibody binding. Targets to which antibodies can be directed include receptors on cancer cells and their ligands or counter-receptors (e.g., CD3, CD19, CD20, CD22, CD30, CD33, CD34, CD40, CD44, CD52, CD70, CD79a, CD123, Her-2, EphA2, lymphocyte associated antigen 1, VEGF or VEGFR, CTLA-4, LIV-1, nectin-4, CD74, and SLTRK-6).

In some aspects, the antibody comprises brentuximab or brentuximab vedotin, anti-CD30, alemtuzumab, anti-CD52, rituximab, anti-CD20, trastuzumab Her/neu, nimotuzumab, cetuximab, anti-EGFR, bevacizumab, anti-VEGF, palivizumab, anti-RSV, abciximab, GpIIb/IIIa, infliximab, adalimumab, certolizumab, golimumab TNF-alpha, baciliximab, daclizumab, anti-IL-2, omalizumab, anti-IgE, gemtuzumab or vadastuximab, anti-CD33, natalizumab, anti-VLA-4, vedolizumab alpha4beta7, belimumab, anti-BAFF, otelixizumab, teplizumab, anti-CD3, ofatumumab, ocrelizumab, epratuzumab, anti-CD22, alemtuzumumab, eculizumab, canakimumab, mepolizumab, reslizumab, tocilizumab, ustekinumab, and briakinumab.

Further aspects are described in WO2018107125, which is herein incorporated by reference.

### B. Collagen Binding Domain

Collagen is an extracellular matrix (ECM)-protein that regulates a variety of cellular biological functions, such as proliferation, differentiation, and adhesion in both normal and tumor tissue (Ricard-Blum, Cold Spring Harb Perspect Biol 3:a004978, 2011). Collagen is the most abundant protein in the mammalian body and exists in almost all tissues in one or more of 28 isoforms (Ricard-Blum, Cold Spring Harb Perspect Biol 3:a004978, 2011). The blood vessel sub-endothelial space is rich in collagen. Because of its insolubility under physiological conditions, collagen barely exists within the blood (Dubois et al., Blood 107:3902-06, 2006; Bergmeier and Hynes, Cold Spring Harb Perspect Biol 4:a005132, 2012). Tumor vasculature is reported to be permeable due to an abnormal structure (Nagy et al., British journal of cancer 100:865, 2009). Thus, with its leaky vasculature, collagen is exposed in the tumor (Liang et al., Journal of controlled release 209:101-109, 2015; Liang et al., Sci Rep 6:18205, 2016; Yasunaga et al., Bioconjugate chemistry 22:1776-83, 2011; Xu et al. The Journal of cell biology 154:1069-80, 2001; Swartz and Lund, Nat Rev Cancer 12:210-19). Also, tumor tissue contains increased amounts of collagen compared to normal tissues (Zhou et al. J Cancer 8:1466-76, 2017; Provenzano et al. BMC Med 6:11, 2008).

von Willebrand factor (vWF) is a blood coagulation factor and binds to both type I and type III collagen, and the adhesion receptor GPIb on blood platelets (Lenting et al., Journal of thrombosis and haemostasis:JTH 10:2428-37, 2012; Shahidi Advances in experimental medicine and biology 906:285-306, 2017). When injured, collagen beneath endothelial cells is exposed to blood plasma, and vWF-collagen binding initiates the thrombosis cascade (Shahidi Advances in experimental medicine and biology 906:285-306, 2017; Wu et al. Blood 99:3623-28, 2002). The vWF A domain has the highest affinity against collagen among reported non-bacterial origin proteins/peptides (Addi et al., Tissue Engineering Part B: Reviews, 2016). Particularly within the A domain, the A3 domain of vWF has been reported as a collagen binding domain (CBD) (Ribba et al. Thrombosis and haemostasis 86:848-54, 2001). As described above, the inventors contemplated that a fusion protein with the vWF A3 CBD may achieve targeted cytokine immunotherapy even when injected systemically due to exposure of collagen via the leaky tumor vasculature.

In some aspects, the collagen binding domain comprises a polypeptide from decorin. Exemplary decorin polypeptides include human decorin, or a fragment thereof, which is represented by the following sequence: CGPFQQRGLFDFMLEDEASGIGPEVPDDRDFEPSLGPVCPFRCQCHLRVVQCSDLGLDK VPKDLPPDTTLLDLQNNKITEIKDGDFKNLKNLHALILVNNKISKVSPGAFTPLVKLERL YLSKNQLKELPEKMPKTLQELRAHENEITKVRKVTFNGLNQMIVIELGTNPLKSSGIENG AFQGMKKLSYIRIADTNITSIPQGLPPSLTELHLDGNKISRVDAASLKGLNNLAKLGLSFN SISAVDNGSLANTPHLRELHLDNNKLTRVPGGLAEHKYIQVVYLHNNNISVVGSSDFCPP GHNTKKASYSGVSLFSNPVQYWEIQPSTFRCVYVRSAIQLGNYK (SEQ ID NO:40), a peptide derived from human decorin: LRELHLDNNC (SEQ ID NO:41), and a peptide derived from bovine decorin: LRELHLNNNC (SEQ ID NO:44).

In some aspects, the CBD comprises a polypeptide fragment from vWF. In some aspects, the CBD comprises vWF A1 derived from human sequence, residues 1237-1458 (474-695 of mature VWF) or a fragment thereof, which is represented by the amino acid sequence:

(SEQ ID NO: 39)
CQEPGGLVVPPTDAPVSPTTLYVEDISEPPLHDFYCSRLLDLVFLLDGS
SRLSEAEFEVLKAFVVDMMERLRISQKWVRVAVVEYHDGSHAYIGLKDR
KRPSELRRIASQVKYAGSQVASTSEVLKYTLFQIFSKIDRPEASRITLL
LMASQEPQRMSRNFVRYVQGLKKKKVIVIPVGIGPHANLKQIRLIEKQA
PENKAFVLSSVDELEQQRDEIVSYLC.

In some aspects, the CBP comprises all or a fragment of vWF A3, which is represented by the following amino acid sequences:

(SEQ ID NO: 37)
CSQPLDVILLLDGSSSFPASYFDEMKSFAKAFISKANIGPRLTQVSVLQ
YGSITTIDVPWNVVPEKAHLLSLVDVMQREGGPSQIGDALGFAVRYLTS
EMHGARPGASKAVVILVTDVSVDSVDAAADAARSNRVTVFPIGIGDRYD
AAQLRILAGPAGDSNVVKLQRIEDLPTMVTLGNSFLHKLCSGFVRICTG
and
(SEQ ID NO: 45)
CSQPLDVILLLDGSSSFPASYFDEMKSFAKAFISKANIGPRLTQVSVLQ
YGSITTIDVPWNVVPEKAHLLSLVDVMQREGGPSQIGDALGFAVRYLTS
EMHGARPGASKAVVILVTDVSVDSVDAAADAARSNRVTVFPIGIGDRYD
AAQLRILAGPAGDSNVVKLQRIEDLPTMVTLGNSFLHKLCSGFVRI.

In some aspects, the CBP comprises vWF A3 domain polypeptide with a 6H tag (SEQ ID NO:87) with the following amino acid sequence:

(SEQ ID NO: 1)
CSQPLDVILLLDGSSSFPASYFDEMKSFAKAFISKANIGPRLTQVSVLQ
YGSITTIDVPWNVVPEKAHLLSLVDVMQREGGPSQIGDALGFAVRYLTS
EMHGARPGASKAVVILVTDVSVDSVDAAADAARSNRVTVFPIGIGDRYD
AAQLRILAGPAGDSNVVKLQRIEDLPTMVTLGNSFLHKLCSGFVRICTG
HHHHHH.

In some aspects, the CBP comprises a peptide or polypeptide from von Willebrand factor (vWF), such as a collagen binding peptide from vWF. The sequence of human vWF comprises the following:

(SEQ ID NO: 38)
MIPARFAGVLLALALILPGTLCAEGTRGRSSTARCSLFGSDFVNTFDGSMYSFAGYCSYL
LAGGCQKRSFSIIGDFQNGKRVSLSVYLGEFFDIHLFVNGTVTQGDQRVSMPYASKGLY
LETEAGYYKLSGEAYGFVARIDGSGNFQVLLSDRYFNKTCGLCGNFNIFAEDDFMTQEG
TLTSDPYDFANSWALSSGEQWCERASPPSSSCNISSGEMQKGLWEQCQLLKSTSVFARC
HPLVDPEPFVALCEKTLCECAGGLECACPALLEYARTCAQEGMVLYGWTDHSACSPVC
PAGMEYRQCVSPCARTCQSLHINEMCQERCVDGCSCPEGQLLDEGLCVESTECPCVHSG
KRYPPGTSLSRDCNTCICRNSQWICSNEECPGECLVTGQSHFKSFDNRYFTFSGICQYLLA
RDCQDHSFSIVIETVQCADDRDAVCTRSVTVRLPGLHNSLVKLKHGAGVAMDGQDVQL
PLLKGDLRIQHTVTASVRLSYGEDLQMDWDGRGRLLVKLSPVYAGKTCGLCGNYNGN
QGDDFLTPSGLAEPRVEDFGNAWKLHGDCQDLQKQHSDPCALNPRMTRFSEEACAVLT
SPTFEACHRAVSPLPYLRNCRYDVCSCSDGRECLCGALASYAAACAGRGVRVAWREPG
RCELNCPKGQVYLQCGTPCNLTCRSLSYPDEECNEACLEGCFCPPGLYMDERGDCVPK
AQCPCYYDGEIFQPEDIFSDHHTMCYCEDGFMHCTMSGVPGSLLPDAVLSSPLSHRSKR
SLSCRPPMVKLVCPADNLRAEGLECTKTCQNYDLECMSMGCVSGCLCPPGMVRHENR
CVALERCPCFHQGKEYAPGETVKIGCNTCVCRDRKWNCTDHVCDATCSTIGMAHYLTF
DGLKYLFPGECQYVLVQDYCGSNPGTFRILVGNKGCSHPSVKCKKRVTILVEGGEIELF
DGEVNVKRPMKDETHFEVVESGRYIILLLGKALSVVWDRHLSISVVLKQTYQEKVCGLC
GNFDGIQNNDLTSSNLQVEEDPVDFGNSWKVSSQCADTRKVPLDSSPATCHNNIMKQT
MVDSSCRILTSDVFQDCNKLVDPEPYLDVCIYDTCSCESIGDCACFCDTIAAYAHVCAQ
HGKVVTWRTATLCPQSCEERNLRENGYECEWRYNSCAPACQVTCQHPEPLACPVQCVE
GCHAHCPPGKILDELLQTCVDPEDCPVCEVAGRRFASGKKVTLNPSDPEHCQICHCDVV
NLTCEACQEPGGLVVPPTDAPVSPTTLYVEDISEPPLHDFYCSRLLDLVFLLDGSSRLSEA
EFEVLKAFVVDMMERLRISQKWVRVAVVEYHDGSHAYIGLKDRKRPSELRRIASQVKY
AGSQVASTSEVLKYTLFQIFSKIDRPEASRITLLLMASQEPQRMSRNFVRYVQGLKKKKV
IVIPVGIGPHANLKQIRLIEKQAPENKAFVLSSVDELEQQRDEIVSYLCDLAPEAPPPTLPP
DMAQVTVGPGLLGVSTLGPKRNSMVLDVAFVLEGSDKIGEADFNRSKEFMEEVIQRMD
VGQDSIHVTVLQYSYMVTVEYPFSEAQSKGDILQRVREIRYQGGNRTNTGLALRYLSDH
SFLVSQGDREQAPNLVYMVTGNPASDEIKRLPGDIQVVPIGVGPNANVQELERIGWPNA
PILIQDFETLPREAPDLVLQRCCSGEGLQIPTLSPAPDCSQPLDVILLLDGSSSFPASYFDE
MKSFAKAFISKANIGPRLTQVSVLQYGSITTIDVPWNVVPEKAHLLSLVDVMQREGGPS
QIGDALGFAVRYLTSEMHGARPGASKAVVILVTDVSVDSVDAAADAARSNRVTVFPIGI
GDRYDAAQLRILAGPAGDSNVVKLQRIEDLPTMVTLGNSFLHKLCSGFVRICMDEDGNE
KRPGDVWTLPDQCHTVTCQPDGQTLLKSHRVNCDRGLRPSCPNSQSPVKVEETCGCRW
TCPCVCTGSSTRHIVTFDGQNFKLTGSCSYVLFQNKEQDLEVILHNGACSPGARQGCMK
SIEVKHSALSVELHSDMEVTVNGRLVSVPYVGGNMEVNVYGAIMHEVRFNHLGHIFTF
TPQNNEFQLQLSPKTFASKTYGLCGICDENGANDFMLRDGTVTTDWKTLVQEWTVQRP
GQTCQPILEEQCLVPDSSHCQVLLLPLFAECHKVLAPATFYAICQQDSCHQEQVCEVIAS
YAHLCRTNGVCVDWRTPDFCAMSCPPSLVYNHCEHGCPRHCDGNVSSCGDHPSEGCFC
PPDKVMLEGSCVPEEACTQCIGEDGVQHQFLEAWVPDHQPCQICTCLSGRKVNCTTQPC
PTAKAPTCGLCEVARLRQNADQCCPEYECVCDPVSCDLPPVPHCERGLQPTLTNPGECR
PNFTCACRKEECKRVSPPSCPPHRLPTLRKTQCCDEYECACNCVNSTVSCPLGYLASTAT
NDCGCTTTTCLPDKVCVHRSTIYPVGQFWEEGCDVCTCTDMEDAVMGLRVAQCSQKP
CEDSCRSGFTYVLHEGECCGRCLPSACEVVTGSPRGDSQSSWKSVGSQWASPENPCLIN
ECVRVKEEVFIQQRNVSCPQLEVPVCPSGFQLSCKTSACCPSCRCERMEACMLNGTVIGP
GKTVMIDVCTTCRCMVQVGVISGFKLECRKTTCNPCPLGYKEENNTGECCGRCLPTACT
IQLRGGQIMTLKRDETLQDGCDTHFCKVNERGEYFWEKRVTGCPPFDEHKCLAEGGKI
MKIPGTCCDTCEEPECNDITARLQYVKVGSCKSEVEVDIHYCQGKCASKAMYSIDINDV
QDQCSCCSPTRTEPMQVALHCTNGSVVYHEVLNAMECKCSPRKCSK.

In some aspects, the peptide is from the vWF A3 domain and has the following amino acid sequence (or a fragment thereof):

(SEQ ID NO: 46)
CSGEGLQIPTLSPAPDCSQPLDVILLLDGSSSFPASYFDEMKSFAKAFI
SKANIGPRLTQVSVLQYGSITTIDVPWNVVPEKAHLLSLVDVMQREGGP
SQIGDALGFAVRYLTSEMHGARPGASKAVVILVTDVSVDSVDAAADAAR
SNRVTVFPIGIGDRYDAAQLRILAGPAGDSNVVKLQRIEDLPTMVTLGN
SFLHKLCSG.

The CBP peptide or polypeptide may be a peptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a CBD peptide or fragment of the peptides described above, such as SEQ ID NOS:1, 37-41, and 44-46.

### C. Linker

In some aspects, the polypeptides comprise or further comprise a linker. The linker may be between any two domains of the polypeptide. In some aspects, the polypeptide comprises a linker between the CBP and the cytokine. In some aspects, the polypeptide comprises a linker in between the CBP and the serum polypeptide. In some aspects, the polypeptide comprises a linker in between the masking agent and the cytokine. In some aspects, the polypeptide comprises a linker in between the albumin and the cytokine. In some aspects, the polypeptide comprises a linker in between the therapeutic agent and the masking agent. In some aspects, the polypeptide comprises a linker in between the therapeutic agent and the CBP. In some aspects, the linker comprises one or more tumor-associated protease cleavage sites. A tumor-associated protease cleavage site refers to a cleavage site that is recognized by a protease that is highly upregulated or enriched in the tumor microenvironment. While the tumor-associated protease cleavage site may not be tumor-specific, meaning that the protease is only expressed in the tumor, it is tumor-enriched, meaning that the protease is expressed at a level in the tumor microenvironment that is higher than normal tissues or most normal tissues. In some aspects, the tumor-associated protease cleavage site comprises an amino acid sequence that is recognized and cleaved by a matrix metalloproteinase. For example, the tumor-associated protease cleavage site may be one that is cleaved by MMP1, MMP2, MMP3, MMP7, MMP8, MMP9, MMP10, MMP11, MMP12, MMP13, MMP14, MMP15, MMP16, MMP17, MMP18, MMP19, MMP20, MMP21, MMP23A, MMP23B, MMP24, MMP25, MMP26, MMP27, MMP28, or combinations thereof. In some aspects, the tumor-associated protease cleavage site comprises the MMP-response sequence of SEQ ID NO: 13: GLLSGRSDNH. In some aspects, the tumor-associated protease cleavage site may be one that is cleaved by thrombin. In some aspects, the thrombin-responsive sequence comprises SEQ ID NO:14: LVPRGS.

In some aspects, two polypeptides such as a two of a CBP, serum protein, therapeutic agent, masking agent, and cytokine may be linked through a bifunctional linker. Linkers, such as amino acid or peptidimimetic sequences may be inserted between the peptide and/or antibody sequence. In an aspect, a fynomer domain is joined to a Heavy (H) chain or Light (L) chain immediately after the last amino acid at the amino(NH2)-terminus or the carboxy(C)-terminus of the Heavy (H) chain or the Light (L) chain. Linkers may have one or more properties that include a flexible conformation, an inability to form an ordered secondary structure or a hydrophobic or charged character which could promote or interact with either domain. Examples of amino acids typically found in flexible protein regions may include Gly, Asn and Ser. For example, a suitable peptide linker may be GGGSGGGS (SEQ ID NO:47) or (GGGS-SEQ ID NO:191)n, wherein n=1, 2, 3, 4, 5, 6, 7, 8, 9, or 10 (or any range derivable therein). Other near neutral amino acids, such as Thr and Ala, may also be used in the linker sequence. The length of the linker sequence may vary without significantly affecting the function or activity of the fusion protein (see, e.g., U.S. Pat. No. 6,087,329). Examples of linkers may also include chemical moieties and conjugating agents, such as sulfo-succinimidyl derivatives (sulfo-SMCC, sulfo-SMPB), disuccinimidyl suberate (DSS), disuccinimidyl glutarate (DSG) and disuccinimidyl tartrate (DST). Examples of linkers further comprise a linear carbon chain, such as CN (where N=1-100 carbon atoms, e.g. N=1, 2, 3, 4, 5, 6, 7, 8, 9, 10, 11, 12, 13, 14, or 15, or more). In some aspects, the linker can be a dipeptide linker, such as a valine-citrulline (val-cit), a phenylalanine-lysine (phe-lys) linker, or maleimidocapronic-valine-citruline-p-aminobenzyloxycarbonyl (vc) linker. In some aspects, the linker is sulfosuccinimidyl-4-[N-maleimidomethyl]cyclohexane-1-carboxylate (smcc). Sulfo-smcc conjugation occurs via a maleimide group which reacts with sulfhydryls (thiols, —SH), while its sulfo-NHS ester is reactive toward primary amines (as found in lysine and the protein or peptide N-terminus). Further, the linker may be maleimidocaproyl (mc).

In some aspects, the linker comprises one or more polypeptides that are cleavable by—i.e., are a substrate for—an enzyme (a protease) that is uniquely expressed or overexpressed in a cancer or tumor microenvironment, compared to healthy tissue or organ. Preferably, the enzyme is found in the extracellular environment of a tumor. Examples of such proteases include: aspartate proteases (e.g., renin), fibroblast activation protein (FAP), aspartic cathepsins (e.g., cathepsin D, caspase 1, caspase 2, etc.), cysteine cathepsins (e.g., cathepsin B), cysteine proteases (e.g., legumain), disintegrin/metalloproteinases (ADAMS, e.g., ADAM8, ADAM9), disintegrin/metalloproteinases with thrombospondin motifs (ADAMTS, e.g., ADAMTS1), integral membrane serine proteases (e.g., matriptase 2, MT-SP1/matriptase, TMPRSS2, TMPRSS3, TMPRSS4), kallikrein-related peptidases (KLKs, e.g. KLK4, KLK5), matrix metalloproteases (e.g., MMP-1, MMP-2, MMP-9), and serine proteases (e.g., cathepsin A, coagulation factor proteases such as elastase, plasmin, thrombin, PSA, uPA, Factor Vila, Factor Xa, and HCV NS3/4). Preferably, the protease is fibroblast activation protein (FAP), urokinase-type plasminogen activator (uPA, urokinase), MT-SP1/matriptase, legumain, or a matrix metalloprotease (especially MMP-1, MMP-2, and MMP-9). Those skilled in the art will appreciate that the choice of the enzyme and the corresponding cleavable peptide will depend on the disease to be treated and the protease(s) expressed by the affected tissue or organ.

Further examples of tumor-associated protease sites include LSGRSDNH (SEQ ID NO:49), cleaved by urokinase, matriptase, or legumain; VPLSLYS (SEQ ID NO:50), cleaved by MMP2 or MMP9; PLGLAG (SEQ ID NO:51), cleaved by MMP2; VLVPMAMMAS (SEQ ID NO:52), cleaved by MMP1; XXQARAX (SEQ ID NO:53), wherein X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine, cleaved by Matriptase; and XXQARVX (SEQ ID NO:249), wherein X is alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine, cleaved by Matriptase; AGPR (SEQ ID NO:54), cleaved by matriptase; AANL (SEQ ID NO:55) and PTNL (SEQ ID NO:56), cleaved by Legumain; and TSGRSANP (SEQ ID NO:57).

A linker sequence may be included in the polypeptides of the disclosures. For example, a linker having at least, at most, or exactly 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more amino acids (or any derivable range therein) may separate or be between any two of a CBP, serum protein, therapeutic agent, masking agent, and cytokine.

The linker may comprise a sequence of SEQ ID NO:13, 14, or 47-57 or a peptide with at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:13, 14, or 47-57.

Further examples of tumor-associated protease sites include those in the table below:

| Substrate | SEQ | | Substrate | SEQ | |
|---|---|---|---|---|---|
| Sequence | ID NO | Protease | Sequence | ID NO | Protease |
| YLGRSYKV | 109 | C1s | RPLALWRS | 152 | MMP7 |
| FK | | Cathepsin B | NKSRLGLG | 153 | MMP7/Cathpsin B |
| TSQVNGLN | 110 | Elastase 2 | GPQGIAGQR | 154 | MMP9 |
| APPPVVLL | 111 | Matrilysin (MMP7) | PVGLIG | 155 | MMP2/9 |
| GGGGF | 112 | Cathepsin B | KPVSLSYR | 156 | MMP9 |
| TPEHVVPY | 113 | Endothelin-converting | QPVGINTS | 157 | MMP3 (stromelysin-1) |
| | | enzyme 1 | | | |
| PRSFFRLGK | 114 | Cathepsin D | PLGMTS | 158 | MMP9 |
| PQGRIVGG | 115 | Hepsin | PRALM | 159 | MMP9 |
| EVLLSWAV | 116 | Cathepsin G | GPLPLR | 160 | MT1-MMP |
| AANL | 55 | Legumain | KQLRVVNG | 161 | MT-SP1/ST14/uPA/ |
| | | | | | Hepsin |
| PVSLSYRC | 117 | Cathepsin G | PLGLYA | 162 | Pan-MMP |
| LRELHLDN | 118 | Matrilysin (MMP7) | AFK | | Plasmin |
| AAPV | 119 | Elastase 2 | LVPRGS | 14 | Thrombin |
| IPENFFGV | 120 | Matrilysin (MMP7) | GGR | | Plasmin/TMPRSS2 |
| PRFKIIGG | 121 | Hepsin | HSSKLQL | 163 | PSA |
| MLEDEASG | 122 | Matrilysin (MMP7) | SSKYQ | 164 | PSA |
| KQSRKFVP | 123 | Matriptase(ST14) | GPLGVRGK | 165 | MMP2 |
| RQARVVGG | 124 | Matriptase2/Hepsin | GGGSGGGS | 47 | Control |
| GKAFRRL | 125 | Hk2 | PRGMAS | 166 | MMP9 |
| GPLGLWAQ | 126 | MMP (esp 2/9) | AAALGNVAP | 167 | MMP9 |
| SLGRKIQI | 127 | MASP2 | GGSGRSANA | 168 | uPA |
| PVSLR | 128 | MMP1 | PLGVRG | 169 | MMP2/9 |
| AAATSIAM | 129 | MMP11 (stromelysin- | SGRSANAK | 170 | uPA |
| | | 3) | | | |
| AAGAMFLE | 130 | MMP11 (stromelysin- | GPLGLWAGG | 171 | MMP2 |
| | | 3) | | | |
| EAAAATSI | 131 | MMP11 (stromelysin- | SGRSA | 172 | uPA |
| | | 3 | | | |
| PRHLR | 132 | MMP14 | PLGLYL | 173 | MMP2/9 |
| PRGLRK | 133 | MMP14 | SRRRVNSL | 174 | uPA |
| VPLSLYSG | 134 | MMP (esp 2/9) | GSTFF | 175 | Cathepsin D |
| PRGLRP | 135 | MMP15/16/24/25 | CPGRVVGG | 176 | uPa/tPA |
| PRHLRN | 136 | MMP15/16/24/25 | GTQFF | 177 | Cathepsin D |
| PRWLRS | 137 | MMP15/16/24/25 | PQGLAG | 178 | MMP9 |
| HPVGLLAR | 138 | MMP2 | QVVAG | 179 | Cathepsin B |
| KGPLGVRG | 139 | MMP2 | TYSRSRYL | 180 | uPA |
| CGLDD | 140 | MMP2/9 | NSGRAVTY | 181 | uPA |
| STAVIVSA | 141 | MMP3 (stromelysin-1) | GPQGARGQ | 182 | MMP9 |
| PLGLAG | 51 | MMP2 | PSSRRRVN | 183 | uPA |
| GPLGIAGQ | 142 | MMP2/9 | PLGLYAL | 184 | MMP2/9 |
| PLGLWA | 143 | MMP2 | PMKRLTLG | 185 | Cathepsin B |
| GPLGMLSQ | 144 | MMP2/9 | DDDKIVGG | 186 | Cathepsin B |
| PLGVRGK | 145 | MMP2 | HLVEALYL | 187 | Cathepsin B |
| GPQGIWGQ | 146 | MMP2/9, MT1-MMP | EVDLLIGS | 188 | Cathepsin B |
| AIPVSLR | 147 | MMP2 | PRFKIIGG | 121 | Cathepsin B |
| GPLGLARK | 148 | MMP7 | AVRWLLTA | 189 | MMP9 |
| PQGIAMG | 149 | MMP2 | LSGRSDNH | 49 | uPA/matriptase/legumain |
| VASSSTAV | 150 | MMP3 (stromelysin-1) | GGGRR | 190 | uPA |
| PLGL | 151 | MMP2/9 | | | |

### D. Serum Proteins

In some aspects, the polypeptides of the disclosure are further linked to a serum protein. Serum proteins include, for example, albumin, globulin, and fibrinogen. Globulins include alpha 1 globulins, alpha 2 globulins, beta globulins, and gamma globulins. The albumin may be mouse, human, bovine, or any other homologous albumin protein. In some aspects, the albumin comprises human serum albumin, which is encoded by the ALB gene, and exemplified by the following amino acid sequence:

(SEQ ID NO: 43)
KWVTFISLLFLFSSAYSRGVFRRDAHKSEVAHRFKDLGEENFKALVLIAFAQYLQQCPFE
DHVKLVNEVTEFAKTCVADESAENCDKSLHTLFGDKLCTVATLRETYGEMADCCAKQ
EPERNECFLQHKDDNPNLPRLVRPEVDVMCTAFHDNEETFLKKYLYEIARRHPYFYAPE
LLFFAKRYKAAFTECCQAADKAACLLPKLDELRDEGKASSAKQRLKCASLQKFGERAF
KAWAVARLSQRFPKAEFAEVSKLVTDLTKVHTECCHGDLLECADDRADLAKYICENQD
SISSKLKECCEKPLLEKSHCIAEVENDEMPADLPSLAADFVESKDVCKNYAEAKDVFLG
MFLYEYARRHPDYSVVLLLRLAKTYETTLEKCCAAADPHECYAKVFDEFKPLVEEPQN
LIKQNCELFEQLGEYKFQNALLVRYTKKVPQVSTPTLVEVSRNLGKVGSKCCKHPEAKR
MPCAEDYLSVVLNQLCVLHEKTPVSDRVTKCCTESLVNRRPCFSALEVDETYVPKEFNA
ETFTFHADICTLSEKERQIKKQTALVELVKHKPKATKEQLKAVMDDFAAFVEKCCKAD
DKETCFAEEGKKLVAASQAALGL.

In some aspects, the albumin comprises mouse albumin having the following sequence:

(SEQ ID NO: 42)
EAHKSEIAHRYNDLGEQHFKGLVLIAFSQYLQKCSYDEHAKLVQEVTDFAKTCVADES
AANCDKSLHTLFGDKLCAIPNLRENYGELADCCTKQEPERNECFLQHKDDNPSLPPFER
PEAEAMCTSFKENPTTFMGHYLHEVARRHPYFYAPELLYYAEQYNEILTQCCAEADKES
CLTPKLDGVKEKALVSSVRQRMKCSSMQKFGERAFKAWAVARLSQTFPNADFAEITKL
ATDLTKVNKECCHGDLLECADDRAELAKYMCENQATISSKLQTCCDKPLLKKAHCLSE
VEHDTMPADLPAIAADFVEDQEVCKNYAEAKDVFLGTFLYEYSRRHPDYSVSLLLRLA
KKYEATLEKCCAEANPPACYGTVLAEFQPLVEEPKNLVKTNCDLYEKLGEYGFQNAILV
RYTQKAPQVSTPTLVEAARNLGRVGTKCCTLPEDQRLPCVEDYLSAILNRVCLLHEKTP
VSEHVTKCCSGSLVERRPCFSALTVDETYVPKEFKAETFTFHSDICTLPEKEKQIKKQTAL
AELVKHKPKATAEQLKTVMDDFAQFLDTCCKAADKDTCFSTEGPNLVTRCKDALA.

In some aspects, the serum protein comprises a polypeptide of SEQ ID NO:42 or 43, or a fragment thereof, or a polypeptide with 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to SEQ ID NO:42, 43, or a fragment thereof.

### E. Polypeptide Aspects

Specific polypeptide aspects are exemplified below:

| | |
|---|---|
| mIL12Rß1 | QLGASGPGDGCCVEKTSFPEGASGSPLGPRNLSCYRVSKTDYECSW |
| ThrombinlinkerIL12 | QYDGPEDNVSHVLWCCFVPPNHTHTGQERCRYFSSGPDRTVQFWE |
| p35 | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQALVPRGSGGGSGGGSRVIPVSGPARCLSQSRN |
| | LLKTTDDMVKTAREKLKHYSCTAEDIDHEDITRDQTSTLKTCLPLE |
| | LHKNESCLATRETSSTTRGSCLPPQKTSLMMTLCLGSIYEDLKMYQ |
| | TEFQAINAALQNHNHQQIILDKGMLVAIDELMQSLNHNGETLRQKP |
| | PVGEADPYRVKMKLCILLHAFSTRVVTINRVMGYLSSA (SEQ ID |
| | NO: 7) |
| CBD- | QLGASGPGDGCCVEKTSFPEGASGSPLGPRNLSCYRVSKTDYECSW |
| mIL12Rß1 | QYDGPEDNVSHVLWCCFVPPNHTHTGQERCRYFSSGPDRTVQFWE |
| mbinlinkerIL12 | QDGIPVLSKVNFWVESRLGNRTMKSQKISQYLYNWTKTTPPLGHIK |
| Throp40 | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQALVPRGSGGGSGGGSGGGSGGGSGGGSGGG |
| | SMWELEKDVYVVEVDWTPDAPGETVNLTCDTPEEDDITWTSDQR |
| | HGVIGSGKTLTITVKEFLDAGQYTCHKGGETLSHSHLLLHKKENGI |
| | WSTEILKNFKNKTFLKCEAPNYSGRFTCSWLVQRNMDLKFNIKSSS |
| | SSPDSRAVTCGMASLSAEKVTLDQRDYEKYSVSCQEDVTCPTAEE |
| | TLPIELALEARQQNKYENYSTSFFIRDIIKPDPPKNLQMKPLKNSQV |
| | EVSWEYPDSWSTPHSYFSLKFFVRIQRKKEKMKETEEGCNQKGAF |
| | LVEKTSTEVQCKGGNVCVQAQDRYYNSSCSKWACVPCRVRS (SEQ |
| | ID NO: 8) |
| mIL12Rß1 | QLGASGPGDGCCVEKTSFPEGASGSPLGPRNLSCYRVSKTDYECSW |
| MMPlinkerIL12p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGLLSGRSDNHGGGSGGGSRVIPVSGPARCLS |
| | QSRNLLKTTDDMVKTAREKLKHYSCTAEDIDHEDITRDQTSTLKTC |
| | LPLELHKNESCLATRETSSTTRGSCLPPQKTSLMMTLCLGSIYEDLK |
| | MYQTEFQAINAALQNHNHQQIILDKGMLVAIDELMQSLNHNGETL |
| | RQKPPVGEADPYRVKMKLCILLHAFSTRVVTINRVMGYLSSAHHH |
| | HHH (SEQ ID NO: 9) |
| mIL12Rß1- | CCVEKTSFPEGASGSPLGPRNLSCYRVSKTDYECSWQYDGPEDNVS |
| MMPlinker- | HVLWCCFVPPNHTHTGQERCRYFSSGPDRTVQFWEQDGIPVLSKV |
| IL12p40 | NFWVESRLGNRTMKSQKISQYLYNWTKTTPPLGHIKVSQSHRQLR |
| | MDWNVSEEAGAEVQFRRRMPTTNWTLGDCGPQVNSGSGVLGDIR |
| | GSMSESCLCPSENMAQEIQIRRRRRLSSGAPGGPWSDWSMPVCVPP |
| | EVLPQAGLLSGRSDNHGGGSGGGSGGGSGGGSGGGSGGGSMWEL |
| | EKDVYVVEVDWTPDAPGETVNLTCDTPEEDDITWTSDQRHGVIGS |
| | GKTLTITVKEFLDAGQYTCHKGGETLSHSHLLLHKKENGIWSTEIL |
| | KNFKNKTFLKCEAPNYSGRFTCSWLVQRNMDLKFNIKSSSSSPDSR |
| | AVTCGMASLSAEKVTLDQRDYEKYSVSCQEDVTCPTAEETLPIELA |
| | LEARQQNKYENYSTSFFIRDIIKPDPPKNLQMKPLKNSQVEVSWEY |
| | PDSWSTPHSYFSLKFFVRIQRKKEKMKETEEGCNQKGAFLVEKTST |
| | EVQCKGGNVCVQAQDRYYNSSCSKWACVPCRVRS (SEQ ID |
| | NO: 10) |
| CBD- | CSQPLDVILLLDGSSSFPASYFDEMKSFAKAFISKANIGPRLTQVSVL |
| mIL12Rß1 | QYGSITTIDVPWNVVPEKAHLLSLVDVMQREGGPSQIGDALGFAV |
| Thrombinlinker- | RYLTSEMHGARPGASKAVVILVTDVSVDSVDAAADAARSNRVTVF |
| IL12p40 | PIGIGDRYDAAQLRILAGPAGDSNVVKLQRIEDLPTMVTLGNSFLH |
| | KLCSGFVRIGGGSGGGSQLGASGPGDGCCVEKTSFPEGASGSPLGP |
| | RNLSCYRVSKTDYECSWQYDGPEDNVSHVLWCCFVPPNHTHTGQ |
| | ERCRYFSSGPDRTVQFWEQDGIPVLSKVNFWVESRLGNRTMKSQK |
| | ISQYLYNWTKTTPPLGHIKVSQSHRQLRMDWNVSEEAGAEVQFRR |
| | RMPTTNWTLGDCGPQVNSGSGVLGDIRGSMSESCLCPSENMAQEI |
| | QIRRRRRLSSGAPGGPWSDWSMPVCVPPEVLPQALVPRGSGGGSG |
| | GGSGGGSGGGSGGGSGGGSMWELEKDVYVVEVDWTPDAPGETV |
| | NLTCDTPEEDDITWTSDQRHGVIGSGKTLTITVKEFLDAGQYTCHK |
| | GGETLSHSHLLLHKKENGIWSTEILKNFKNKTFLKCEAPNYSGRFT |
| | CSWLVQRNMDLKFNIKSSSSSPDSRAVTCGMASLSAEKVTLDQRD |
| | YEKYSVSCQEDVTCPTAEETLPIELALEARQQNKYENYSTSFFIRDII |
| | KPDPPKNLQMKPLKNSQVEVSWEYPDSWSTPHSYFSLKFFVRIQRK |
| | KEKMKETEEGCNQKGAFLVEKTSTEVQCKGGNVCVQAQDRYYNS |
| | SCSKWACVPCRVRS (SEQ ID NO: 11) |
| CBD- | CSQPLDVILLLDGSSSFPASYFDEMKSFAKAFISKANIGPRLTQVSVL |
| mIL12Rß1- | QYGSITTIDVPWNVVPEKAHLLSLVDVMQREGGPSQIGDALGFAV |
| MMPlinker- | RYLTSEMHGARPGASKAVVILVTDVSVDSVDAAADAARSNRVTVF |
| IL12p40 | PIGIGDRYDAAQLRILAGPAGDSNVVKLQRIEDLPTMVTLGNSFLH |
| | KLCSGFVRIGGGSGGGSQLGASGPGDGCCVEKTSFPEGASGSPLGP |
| | RNLSCYRVSKTDYECSWQYDGPEDNVSHVLWCCFVPPNHTHTGQ |
| | ERCRYFSSGPDRTVQFWEQDGIPVLSKVNFWVESRLGNRTMKSQK |
| | ISQYLYNWTKTTPPLGHIKVSQSHRQLRMDWNVSEEAGAEVQFRR |
| | RMPTTNWTLGDCGPQVNSGSGVLGDIRGSMSESCLCPSENMAQEI |
| | QIRRRRRLSSGAPGGPWSDWSMPVCVPPEVLPQAGLLSGRSDNHG |
| | GGSGGGSGGGSGGGSGGGSGGGSMWELEKDVYVVEVDWTPDAP |
| | GETVNLTCDTPEEDDITWTSDQRHGVIGSGKTLTITVKEFLDAGQY |
| | TCHKGGETLSHSHLLLHKKENGIWSTEILKNFKNKTFLKCEAPNYS |
| | GRFTCSWLVQRNMDLKFNIKSSSSSPDSRAVTCGMASLSAEKVTLD |
| | QRDYEKYSVSCQEDVTCPTAEETLPIELALEARQQNKYENYSTSFFI |
| | RDIIKPDPPKNLQMKPLKNSQVEVSWEYPDSWSTPHSYFSLKFFVRI |
| | QRKKEKMKETEEGCNQKGAFLVEKTSTEVQCKGGNVCVQAQDRY |
| | YNSSCSKWACVPCRVRS (SEQ ID NO: 12) |
| mIL12Rß1- | QLGASGPGDGCCVEKTSFPEGASGSPLGPRNLSCYRVSKTDYECSW |
| Thrombin- | QYDGPEDNVSHVLWCCFVPPNHTHTGQERCRYFSSGPDRTVQFWE |
| MMP- | QDGIPVLSKVNFWVESRLGNRTMKSQKISQYLYNWTKTTPPLGHIK |
| MMPlinker- | VSQSHRQLRMDWNVSEEAGAEVQFRRRMPTTNWTLGDCGPQVNS |
| IL12p35 | GSGVLGDIRGSMSESCLCPSENMAQEIQIRRRRRLSSGAPGGPWSD |
| | WSMPVCVPPEVLPQALVPRGSGLLSGRSDNHGLLSGRSDNHGGGS |
| | GGGSRVIPVSGPARCLSQSRNLLKTTDDMVKTAREKLKHYSCTAE |
| | DIDHEDITRDQTSTLKTCLPLELHKNESCLATRETSSTTRGSCLPPQK |
| | TSLMMTLCLGSIYEDLKMYQTEFQAINAALQNHNHQQIILDKGML |
| | VAIDELMQSLNHNGETLRQKPPVGEADPYRVKMKLCILLHAFSTR |
| | VVTINRVMGYLSSA (SEQ ID NO: 15) |
| mIL12Rß1 | CCVEKTSFPEGASGSPLGPRNLSCYRVSKTDYECSWQYDGPEDNVS |
| MMPMMPMMP | HVLWCCFVPPNHTHTGQERCRYFSSGPDRTVQFWEQDGIPVLSKV |
| linkerIL12p35 | NFWVESRLGNRTMKSQKISQYLYNWTKTTPPLGHIKVSQSHRQLR |
| | MDWNVSEEAGAEVQFRRRMPTTNWTLGDCGPQVNSGSGVLGDIR |
| | GSMSESCLCPSENMAQEIQIRRRRRLSSGAPGGPWSDWSMPVCVPP |
| | EVLPQAGLLSGRSDNHGLLSGRSDNHGLLSGRSDNHGGGSGGGSR |
| | VIPVSGPARCLSQSRNLLKTTDDMVKTAREKLKHYSCTAEDIDHED |
| | ITRDQTSTLKTCLPLELHKNESCLATRETSSTTRGSCLPPQKTSLMM |
| | TLCLGSIYEDLKMYQTEFQAINAALQNHNHQQIILDKGMLVAIDEL |
| | MQSLNHNGETLRQKPPVGEADPYRVKMKLCILLHAFSTRVVTINR |
| | VMGYLSSA (SEQ ID NO: 16) |
| mIL12RB1- | CCVEKTSFPEGASGSPLGPRNLSCYRVSKTDYECSWQYDGPEDNVS |
| Thrombin- | HVLWCCFVPPNHTHTGQERCRYFSSGPDRTVQFWEQDGIPVLSKV |
| Thrombin- | NFWVESRLGNRTMKSQKISQYLYNWTKTTPPLGHIKVSQSHRQLR |
| Thrombin- | MDWNVSEEAGAEVQFRRRMPTTNWTLGDCGPQVNSGSGVLGDIR |
| linker-IL12p35 | GSMSESCLCPSENMAQEIQIRRRRRLSSGAPGGPWSDWSMPVCVPP |
| | EVLPQALVPRGSLVPRGSLVPRGSGGGSGGGSRVIPVSGPARCLSQS |
| | RNLLKTTDDMVKTAREKLKHYSCTAEDIDHEDITRDQTSTLKTCLP |
| | LELHKNESCLATRETSSTTRGSCLPPQKTSLMMTLCLGSIYEDLKM |
| | YQTEFQAINAALQNHNHQQIILDKGMLVAIDELMQSLNHNGETLR |
| | QKPPVGEADPYRVKMKLCILLHAFSTRVVTINRVMGYLSSA (SEQ |
| | ID NO: 17) |
| mIL12RB1- | CCVEKTSFPEGASGSPLGPRNLSCYRVSKTDYECSWQYDGPEDNVS |
| Throm3- | HVLWCCFVPPNHTHTGQERCRYFSSGPDRTVQFWEQDGIPVLSKV |
| MMP3-linker- | NFWVESRLGNRTMKSQKISQYLYNWTKTTPPLGHIKVSQSHRQLR |
| IL12p35 | MDWNVSEEAGAEVQFRRRMPTTNWTLGDCGPQVNSGSGVLGDIR |
| | GSMSESCLCPSENMAQEIQIRRRRRLSSGAPGGPWSDWSMPVCVPP |
| | EVLPQALVPRGSLVPRGSLVPRGSGLLSGRSDNHGLLSGRSDNHGL |
| | LSGRSDNHGGGSGGGSRVIPVSGPARCLSQSRNLLKTTDDMVKTA |
| | REKLKHYSCTAEDIDHEDITRDQTSTLKTCLPLELHKNESCLATRET |
| | SSTTRGSCLPPQKTSLMMTLCLGSIYEDLKMYQTEFQAINAALQNH |
| | NHQQIILDKGMLVAIDELMQSLNHNGETLRQKPPVGEADPYRVKM |
| | KLCILLHAFSTRVVTINRVMGYLSSA (SEQ ID NO: 18) |
| VWF A3 | CSQPLDVILLLDGSSSFPASYFDEMKSFAKAFISKANIGPRLTQVSVL |
| domain protein | QYGSITTIDVPWNVVPEKAHLLSLVDVMQREGGPSQIGDALGFAVR |
| | YLTSEMHGARPGASKAVVILVTDVSVDSVDAAADAARSNRVTVFPI |
| | GIGDRYDAAQLRILAGPAGDSNVVKLQRIEDLPTMVTLGNSFLHKL |
| | CSGFVRICTGHHHHHH (SEQ ID NO: 1) |
| FN12mIL- | QLGASGPGDGCCVEKTSFPEGASGSPLGPRNLSCYRVSKTDYECSW |
| 12Rß1 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQALVPRGS (SEQ ID NO: 2) |
| Human IL-12 | p35 Subunit: |
| | RNLPVATPDPGMFPCLHHSQNLLRAVSNMLQKARQTLEFYPCTSEEI |
| | DHEDITKDKTSTVEACLPLELTKNESCLNSRETSFITNGSCLASRKTS |
| | FMMALCLSSIYEDLKMYQVEFKTMNAKLLMDPKRQIFLDQNMLA |
| | VIDELMQALNFNSETVPQKSSLEEPDFYKTKIKLCILLHAFRIRAVTI |
| | DRVMSYLNAS (SEQ ID NO: 3) |
| | p40 Subunit: |
| | IWELKKDVYVVELDWYPDAPGEMVVLTCDTPEEDGITWTLDQSSE |
| | VLGSGKTLTIQVKEFGDAGQYTCHKGGEVLSHSLLLLHKKEDGIWS |
| | TDILKDQKEPKNKTFLRCEAKNYSGRFTCWWLTTISTDLTFSVKSSR |
| | GSSDPQGVTCGAATLSAERVRGDNKEYEYSVECQEDSACPAAEESL |
| | PIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVI |
| | CRKNASISVRAQDRYYSSSWSEWASVPCS (SEQ ID NO: 4) |
| Mouse IL-12 | p35 Subunit: |
| | RVIPVSGPARCLSQSRNLLKTTDDMVKTAREKLKHYSCTAEDIDHE |
| | DITRDQTSTLKTCLPLELHKNESCLATRETSSTTRGSCLPPQKTSLM |
| | MTLCLGSIYEDLKMYQTEFQAINAALQNHNHQQIILDKGMLVAIDE |
| | LMQSLNHNGETLRQKPPVGEADPYRVKMKLCILLHAFSTRVVTINR |
| | VMGYLSSA (SEQ ID NO: 5) |
| | p40 Subunit: |
| | MWELEKDVYVVEVDWTPDAPGETVNLTCDTPEEDDITWTSDQRH |
| | GVIGSGKTLTITVKEFLDAGQYTCHKGGETLSHSHLLLHKKENGIW |
| | STEILKNFKNKTFLKCEAPNYSGRFTCSWLVQRNMDLKFNIKSSSSS |
| | PDSRAVTCGMASLSAEKVTLDQRDYEKYSVSCQEDVTCPTAEETLP |
| | IELALEARQQNKYENYSTSFFIRDIIKPDPPKNLQMKPLKNSQVEVS |
| | WEYPDSWSTPHSYFSLKFFVRIQRKKEKMKETEEGCNQKGAFLVE |
| | KTSTEVQCKGGNVCVQAQDRYYNSSCSKWACVPCRVRS (SEQ ID |
| | NO: 6) |
| uPA responsive | GLLSGRSDNH (SEQ ID NO: 13) |
| sequence (LS) | |
| MMP | HPVGLLAR (SEQ ID NO: 138) |
| responsive | |
| sequence (HP) | |
| MMP | VPLSLYSG (SEQ ID NO: 134) |
| responsive | |
| sequence (VP) | |
| MMP | PLGVRGK (SEQ ID NO: 145) |
| responsive | |
| sequence (PL) | |
| MMP | GERGPPGPQGAARGFPGTPGL (SEQ ID NO: 194) |
| responsive | |
| sequence | |
| (GER) | |
| Human IL- | CRTSECCFQDPPYPDADSGSASGPRDLRCYRISSDRYECSWQYEGP |
| 12Rß1 | TAGVSHFLRCCLSSGRCCYFAAGSATRLQFSDQAGVSVLYTVTLWV |
| | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPENPPQPQVRFSVEQLGQD |
| | GRRRLTLKEQPTQLELPEGCQGLAPGTEVTYRLQLHMLSCPCKAK |
| | ATRTLHLGKMPYLSGAAYNVAVISSNQFGPGLNQTWHIPADTHTEP |
| | VALNISVGTNGTTMYWPARAQSMTYCIEWQPVGQDGGLATCSLTA |
| | PQDPDPAGMATYSWSRESGAMGQEKCYYITIFASAHPEKLTLWSTV |
| | LSTYHFGGNASAAGTPHHVSVKNHSLDSVSVDWAPSLLSTCPGVL |
| | KEYVVRCRDEDSKQVSEHPVQPTETQVTLSGLRAGVAYTVQVRAD |
| | TAWLRGVWSQPQRFSIEVQVSDWLIFFASLGSFLSILLVGVLGYLGL |
| | NRAARHLCPPLPTPCASSAIEFPGGKETWQWINPVDFQEEASLQEA |
| | LVVEMSWDKGERTEPLEKTELPEGAPELALDTELSLEDGDRCKAK |
| | M (SEQ ID NO: 19) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1 | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPE (SEQ ID NO: 195) |
| Mouse IL- | QLGASGPGDGCCVEKTSFPEGASGSPLGPRNLSCYRVSKTDYECSW |
| 12Rß1 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAKIKFLVEPLNQGGRRRLTMQGQSPQLAVPEG |
| | CRGRPGAQVKKHLVLVRMLSCRCQAQTSKTVPLGKKLNLSGATYD |
| | LNVLAKTRFGRSTIQKWHLPAQELTETRALNVSVGGNMTSMQWA |
| | AQAPGTTYCLEWQPWFQHRNHTHCTLIVPEEEDPAKMVTHSWSSK |
| | PTLEQEECYRITVFASKNPKNPMLWATVLSSYYFGGNASRAGTPRH |
| | VSVRNQTGDSVSVEWTASQLSTCPGVLTQYVVRCEAEDGAWESE |
| | WLVPPTKTQVTLDGLRSRVMYKVQVRADTARLPGAWSHPQRFSFE |
| | VQISRLSIIFASLGSFASVLLVGSLGYIGLNRAAWHLCPPLPTPCGSTA |
| | VEFPGSQGKQAWQWCNPEDFPEVLYPRDALVVEMPGDRGDGTESP |
| | QAAPECALDTRRPLETQRQRQVQALSEARRLGLAREDCPRGDLAH |
| | VTLPLLLGGVTQGASVLDDLWRTHKTAEPGPPTLGQEA (SEQ ID |
| | NO: 20) |
| Human IL- | KIDACKRGDVTVKPSHVILLGSTVNITCSLKPRQGCFHYSRRNKLIL |
| 12Rß2 | YKFDRRINFHHGHSLNSQVTGLPLGTTLFVCKLACINSDEIQICGAEI |
| | FVGVAPEQPQNLSCIQKGEQGTVACTWERGRDTHLYTEYTLQLSGP |
| | KNLTWQKQCKDIYCDYLDFGINLTPESPESNFTAKVTAVNSLGSSSS |
| | LPSTFTFLDIVRPLPPWDIRIKFQKASVSRCTLYWRDEGLVLLNRLR |
| | YRPSNSRLWNMVNVTKAKGRHDLLDLKPFTEYEFQISSKLHLYKG |
| | SWSDWSESLRAQTPEEEPTGMLDVWYMKRHIDYSRQQISLFWKNL |
| | SVSEARGKILHYQVTLQELTGGKAMTQNITGHTSWTTVIPRTGNWA |
| | VAVSAANSKGSSLPTRINIMNLCEAGLLAPRQVSANSEGMDNILVT |
| | WQPPRKDPSAVQEYVVEWRELHPGGDTQVPLNWLRSRPYNVSALI |
| | SENIKSYICYEIRVYALSGDQGGCSSILGNSKHKAPLSGPHINAITEE |
| | KGSILISWNSIPVQEQMGCLLHYRIYWKERDSNSQPQLCEIPYRVSQ |
| | NSHPINSLQPRVTYVLWMTALTAAGESSHGNEREFCLQGKANWMA |
| | FVAPSICIAIIMVGIFSTHYFQQKVFVLLAALRPQWCSREIPDPANST |
| | CAKKYPIAEEKTQLPLDRLLIDWPTPEDPEPLVISEVLHQVTPVFRHP |
| | PCSNWPQREKGIQGHQASEKDMMHSASSPPPPRALQAESRQLVDL |
| | YKVLESRGSDPKPENPACPWTVLPAGDLPTHDGYLPSNIDDLPSHE |
| | APLADSLEELEPQHISLSVFPSSSLHPLTFSCGDKLTLDQLKMRCDSL |
| | ML (SEQ ID NO: 21) |
| Mouse IL- | NIDVCKLGTVTVQPAPVIPLGSAANISCSLNPKQGCSHYPSSNELILL |
| 12Rß2 | KFVNDVLVENLHGKKVHDHTGHSSTFQVTNLSLGMTLFVCKLNCS |
| | NSQKKPPVPVCGVEISVGVAPEPPQNISCVQEGENGTVACSWNSGK |
| | VTYLKTNYTLQLSGPNNLTCQKQCFSDNRQNCNRLDLGINLSPDLA |
| | ESRFIVRVTAINDLGNSSSLPHTFTFLDIVIPLPPWDIRINFLNASGSR |
| | GTLQWEDEGQVVLNQLRYQPLNSTSWNMVNATNAKGKYDLRDL |
| | RPFTEYEFQISSKLHLSGGSWSNWSESLRTRTPEEEPVGILDIWYMK |
| | QDIDYDRQQISLFWKSLNPSEARGKILHYQVTLQEVTKKTTLQNTT |
| | RHTSWTRVIPRTGAWTASVSAANSKGASAPTHINIVDLCGTGLLAP |
| | HQVSAKSENMDNILVTWQPPKKADSAVREYIVEWRALQPGSITKFP |
| | PHWLRIPPDNMSALISENIKPYICYEIRVHALSESQGGCSSIRGDSKH |
| | KAPVSGPHITAITEKKERLFISWTHIPFPEQRGCILHYRIYWKERDST |
| | AQPELCEIQYRRSQNSHPISSLQPRVTYVLWMTAVTAAGESPQGNER |
| | EFCPQGKANWKAFVISSICIAIITVGTFSIRYFRQKAFTLLSTLKPQW |
| | YSRTIPDPANSTWVKKYPILEEKIQLPTDNLLMAWPTPEEPEPLIIHE |
| | VLYHMIPVVRQPYYFKRGQGFQGYSTSKQDAMYIANPQATGTLTA |
| | ETRQLVNLYKVLESRDPDSKLANLTSPLTVTPVNYLPSHEGYLPSNI |
| | EDLSPHEADPTDSFDLEHQHISLSIFASSSLRPLIFGGERLTLDRLKM |
| | GYDSLMSNEA (SEQ ID NO: 22) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| (GGGS) | TPDNQVGAEVQFRHRTPSSPWKLGDCGPQDDDTESCLCPLEMNVA |
| | QEFQLRRRQLGSQGSSWSKWSSPVCVPPEGGGSGGGSGGGSGGGS |
| | GGGSGGGSGGGSGGGSGGGSGGGSGGGSGRNLPVATPDPGMFPCL |
| | HHSQNLLRAVSNMLQKARQTLEFYPCTSEEIDHEDITKDKTSTVEA |
| | CLPLELTKNESCLNSRETSFITNGSCLASRKTSFMMALCLSSIYEDLK |
| | MYQVEFKTMNAKLLMDPKRQIFLDQNMLAVIDELMQALNFNSET |
| | VPQKSSLEEPDFYKTKIKLCILLHAFRIRAVTIDRVMSYLNAS (SEQ |
| | ID NO: 196) |
| Human | CRTSECCFQDPPYPDADSGSASGPRDLRCYRISSDRYECSWQYEGP |
| FN12rb 1HPVP | TAGVSHFLRCCLSSGRCCYFAAGSATRLQFSDQAGVSVLYTVTLWV |
| 2LS(51)_p35 | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPEHPVGLLARVPLSLYSGH |
| | PVGLLARVPLSLYSGSGLLSGRSDNHGGGSGGGSRNLPVATPDPGM |
| | FPCLHHSQNLLRAVSNMLQKARQTLEFYPCTSEEIDHEDITKDKTST |
| | VEACLPLELTKNESCLNSRETSFITNGSCLASRKTSFMMALCLSSIYE |
| | DLKMYQVEFKTMNAKLLMDPKRQIFLDQNMLAVIDELMQALNFN |
| | SETVPQKSSLEEPDFYKTKIKLCILLHAFRIRAVTIDRVMSYLNAS |
| | (SEQ ID NO: 197) |
| Linker | HPVGLLARVPLSLYSGHPVGLLARVPLSLYSGSGLLSGRSDNH (SEQ |
| | ID NO: 219) |
| Linker | HPVGLLARVPLSLYSGHPVGLLARVPLSLYSGSGLLSGRSDNHGGG |
| | SGGGS (SEQ ID NO: 220) |
| FN12mIL12rb1 | QLGASGPGDCCVEKTSFPEGASGSPLGPRNLSCYRVSKTDYECSWQ |
| HPVP2LSmIL12 | YDGPEDNVSHVLWCCFVPPNHTHTGQERCRYFSSGPDRTVQFWEQ |
| p35 | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAHPVGLLARVPLSYSGHPVGLLARVPLSYSGSG |
| | LLSGRSDNHGGGSGGGSRVIPVSGPARCLSQSRNLLKTTDDMVKTA |
| | REKLKHYSCTAEDIDHEDITRDQTSTLKTCLPLELHKNESCLATRET |
| | SSTTRGSCLPPQKTSLMMTLCLGSIYEDLKMYQTEFQAINAALQNH |
| | NHQQIILDKGMLVAIDELMQSLNHNGETLRQKPPVGEADPYRVKM |
| | KLCILLHAFSTRVVTINRVMGYLSSA (SEQ ID NO: 198) |
| Linker | HPVGLLARVPLSYSGHPVGLLARVPLSYSGSGLLSGRSDNH (SEQ |
| | ID NO: 103) |
| Linker | HPVGLLARVPLSYSGHPVGLLARVPLSYSGSGLLSGRSDNHGGGSG |
| | GGS (SEQ ID NO: 225) |
| FN12mIL12rb1 | QLGASGPGDCCVEKTSFPEGASGSPLGPRNLSCYRVSKTDYECSWQ |
| PL3mIL12p35 | YDGPEDNVSHVLWCCFVPPNHTHTGQERCRYFSSGPDRTVQFWEQ |
| | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAPLGVRGKPLGVRGKPLGVRGKGGGSGGGSRV |
| | IPVSGPARCLSQSRNLLKTTDDMVKTAREKLKHYSCTAEDIDHEDIT |
| | RDQTSTLKTCLPLELHKNESCLATRETSSTTRGSCLPPQKTSLMMTL |
| | CLGSIYEDLKMYQTEFQAINAALQNHNHQQIILDKGMLVAIDELMQ |
| | SLNHNGETLRQKPPVGEADPYRVKMKLCILLHAFSTRVVTINRVMG |
| | YLSSA (SEQ ID NO: 199) |
| Linker | PLGVRGKPLGVRGKPLGVRGK (SEQ ID NO: 104) |
| Linker | PLGVRGKPLGVRGKPLGVRGKGGGSGGGS (SEQ ID NO: 226) |
| FN12mIL12rb1 | QLGASGPGDCCVEKTSFPEGASGSPLGPRNLSCYRVSKTDYECSWQ |
| GER2mIL12p35 | YDGPEDNVSHVLWCCFVPPNHTHTGQERCRYFSSGPDRTVQFWEQ |
| | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAGERGPPGPQGAARGFPGTPGLGERGPPGPQGA |
| | ARGFPGTPGLGGGSGGGSRVIPVSGPARCLSQSRNLLKTTDDMVKT |
| | AREKLKHYSCTAEDIDHEDITRDQTSTLKTCLPLELHKNESCLATRE |
| | TSSTTRGSCLPPQKTSLMMTLCLGSIYEDLKMYQTEFQAINAALQN |
| | HNHQQIILDKGMLVAIDELMQSLNHNGETLRQKPPVGEADPYRVK |
| | MKLCILLHAFSTRVVTINRVMGYLSSA (SEQ ID NO: 200) |
| Linker | GERGPPGPQGAARGFPGTPGLGERGPPGPQGAARGFPGTPGL (SEQ |
| | ID NO: 105) |
| Linker | GERGPPGPQGAARGFPGTPGLGERGPPGPQGAARGFPGTPGLGGGS |
| | GGGS (SEQ ID NO: 227) |
| FN12mIL12rb1 | QLGASGPGDCCVEKTSFPEGASGSPLGPRNLSCYRVSKTDYECSWQ |
| PL3GER2mIL | YDGPEDNVSHVLWCCFVPPNHTHTGQERCRYFSSGPDRTVQFWEQ |
| 12p35 | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAPLGVRGKPLGVRGKPLGVRGKGERGPPGPQG |
| | AARGFPGTPGLGERGPPGPQGAARGFPGTPGLGGGSGGGSRVIPVS |
| | GPARCLSQSRNLLKTTDDMVKTAREKLKHYSCTAEDIDHEDITRDQ |
| | TSTLKTCLPLELHKNESCLATRETSSTTRGSCLPPQKTSLMMTLCLG |
| | SIYEDLKMYQTEFQAINAALQNHNHQQIILDKGMLVAIDELMQSLN |
| | HNGETLRQKPPVGEADPYRVKMKLCILLHAFSTRVVTINRVMGYLS |
| | SA (SEQ ID NO: 201) |
| Linker | PLGVRGKPLGVRGKPLGVRGKGERGPPGPQGAARGFPGTPGLGER |
| | GPPGPQGAARGFPGTPGL (SEQ ID NO: 106) |
| Linker | PLGVRGKPLGVRGKPLGVRGKGERGPPGPQGAARGFPGTPGLGER |
| | GPPGPQGAARGFPGTPGLGGGSGGGS (SEQ ID NO: 228) |
| FN12mIL12rb1 | QLGASGPGDCCVEKTSFPEGASGSPLGPRNLSCYRVSKTDYECSWQ |
| HPVPLSPL2m | YDGPEDNVSHVLWCCFVPPNHTHTGQERCRYFSSGPDRTVQFWEQ |
| IL12p35 | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAHPVGLLARVPLSYSGHPVGLLARVPLSYSGSG |
| | LLSGRSDNHPLGVRGKPLGVRGKGGGSGGGSRVIPVSGPARCLSQS |
| | RNLLKTTDDMVKTAREKLKHYSCTAEDIDHEDITRDQTSTLKTCLP |
| | LELHKNESCLATRETSSTTRGSCLPPQKTSLMMTLCLGSIYEDLKM |
| | YQTEFQAINAALQNHNHQQIILDKGMLVAIDELMQSLNHNGETLRQ |
| | KPPVGEADPYRVKMKLCILLHAFSTRVVTINRVMGYLSSA (SEQ ID |
| | NO: 202) |
| Linker | PLGVRGKPLGVRGK (SEQ ID NO: 107) |
| Linker | PLGVRGKPLGVRGKGGGSGGGS (SEQ ID NO: 229) |
| FN12mIL12rb1 | QLGASGPGDCCVEKTSFPEGASGSPLGPRNLSCYRVSKTDYECSWQ |
| HPVPLSPLGE | YDGPEDNVSHVLWCCFVPPNHTHTGQERCRYFSSGPDRTVQFWEQ |
| R2IL12p35 | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAHPVGLLARVPLSYSGHPVGLLARVPLSYSGSG |
| | LLSGRSDNHPLGVRGKPLGVRGKGERGPPGPQGAARGFPGTPGLG |
| | ERGPPGPQGAARGFPGTPGLGGGSGGGSRVIPVSGPARCLSQSRNLL |
| | KTTDDMVKTAREKLKHYSCTAEDIDHEDITRDQTSTLKTCLPLELH |
| | KNESCLATRETSSTTRGSCLPPQKTSLMMTLCLGSIYEDLKMYQTE |
| | FQAINAALQNHNHQQIILDKGMLVAIDELMQSLNHNGETLRQKPPV |
| | GEADPYRVKMKLCILLHAFSTRVVTINRVMGYLSSA (SEQ ID |
| | NO: 203) |
| Linker | HPVGLLARVPLSYSGHPVGLLARVPLSYSGSGLLSGRSDNHPLGVR |
| | GKPLGVRGKGERGPPGPQGAARGFPGTPGLGERGPPGPQGAARGF |
| | PGTPGL (SEQ ID NO: 108) |
| Linker | HPVGLLARVPLSYSGHPVGLLARVPLSYSGSGLLSGRSDNHPLGVR |
| | GKPLGVRGKGERGPPGPQGAARGFPGTPGLGERGPPGPQGAARGF |
| | PGTPGLGGGSGGGS (SEQ ID NO: 230) |
| CBD- | CSQPLDVILLLDGSSSFPASYFDEMKSFAKAFISKANIGPRLTQVSVL |
| FN12mIL12rb1 | QYGSITTIDVPWNVVPEKAHLLSLVDVMQREGGPSQIGDALGFAVR |
| HPVPLSIL12p35 | YLTSEMHGARPGASKAVVILVTDVSVDSVDAAADAARSNRVTVFPI |
| | GIGDRYDAAQLRILAGPAGDSNVVKLQRIEDLPTMVTLGNSFLHKL |
| | CSGFVRIGGGSGGGSCCVEKTSFPEGASGSPLGPRNLSCYRVSKTD |
| | YECSWQYDGPEDNVSHVLWCCFVPPNHTHTGQERCRYFSSGPDRT |
| | VQFWEQDGIPVLSKVNFWVESRLGNRTMKSQKISQYLYNWTKTTP |
| | PLGHIKVSQSHRQLRMDWNVSEEAGAEVQFRRRMPTTNWTLGDC |
| | GPQVNSGSGVLGDIRGSMSESCLCPSENMAQEIQIRRRRRLSSGAPG |
| | GPWSDWSMPVCVPPEVLPQAHPVGLLARVPLSYSGSGLLSGRSDN |
| | HGGGSGGGSRVIPVSGPARCLSQSRNLLKTTDDMVKTAREKLKHYS |
| | CTAEDIDHEDITRDQTSTLKTCLPLELHKNESCLATRETSSTTRGSCL |
| | PPQKTSLMMTLCLGSIYEDLKMYQTEFQAINAALQNHNHQQIILDK |
| | GMLVAIDELMQSLNHNGETLRQKPPVGEADPYRVKMKLCILLHAFS |
| | TRVVTINRVMGYLSSA (SEQ ID NO: 204) |
| Linker | HPVGLLARVPLSYSGSGLLSGRSDNH (SEQ ID NO: 221) |
| Linker | HPVGLLARVPLSYSGSGLLSGRSDNHGGGSGGGS (SEQ ID NO: 231) |
| CBDFN12mIL | CSQPLDVILLLDGSSSFPASYFDEMKSFAKAFISKANIGPRLTQVSVL |
| 12rb1HPVP2L | QYGSITTIDVPWNVVPEKAHLLSLVDVMQREGGPSQIGDALGFAVR |
| SIL12p35 | YLTSEMHGARPGASKAVVILVTDVSVDSVDAAADAARSNRVTVFPI |
| | GIGDRYDAAQLRILAGPAGDSNVVKLQRIEDLPTMVTLGNSFLHKL |
| | CSGFVRIGGGSGGGSCCVEKTSFPEGASGSPLGPRNLSCYRVSKTD |
| | YECSWQYDGPEDNVSHVLWCCFVPPNHTHTGQERCRYFSSGPDRT |
| | VQFWEQDGIPVLSKVNFWVESRLGNRTMKSQKISQYLYNWTKTTP |
| | PLGHIKVSQSHRQLRMDWNVSEEAGAEVQFRRRMPTTNWTLGDC |
| | GPQVNSGSGVLGDIRGSMSESCLCPSENMAQEIQIRRRRRLSSGAPG |
| | GPWSDWSMPVCVPPEVLPQAHPVGLLARVPLSYSGHPVGLLARVP |
| | LSYSGSGLLSGRSDNHGGGSGGGSRVIPVSGPARCLSQSRNLLKTTD |
| | DMVKTAREKLKHYSCTAEDIDHEDITRDQTSTLKTCLPLELHKNES |
| | CLATRETSSTTRGSCLPPQKTSLMMTLCLGSIYEDLKMYQTEFQAIN |
| | AALQNHNHQQIILDKGMLVAIDELMQSLNHNGETLRQKPPVGEAD |
| | PYRVKMKLCILLHAFSTRVVTINRVMGYLSSA (SEQ ID NO: 205) |
| Linker | HPVGLLARVPLSYSGHPVGLLARVPLSYSGSGLLSGRSDNH (SEQ |
| | ID NO: 103) |
| Linker | HPVGLLARVPLSYSGHPVGLLARVPLSYSGSGLLSGRSDNHGGGSG |
| | GGS (SEQ ID NO: 225) |
| FN12mIL12rb1 | QLGASGPGDCCVEKTSFPEGASGSPLGPRNLSCYRVSKTDYECSWQ |
| HPVP2LS3IL12 | YDGPEDNVSHVLWCCFVPPNHTHTGQERCRYFSSGPDRTVQFWEQ |
| p35 | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAHPVGLLARVPLSYSGHPVGLLARVPLSYSGSG |
| | LLSGRSDNHISSGLLSGRSDNHISSGLLSGRSDNHGGGSGGGSRVIP |
| | VSGPARCLSQSRNLLKTTDDMVKTAREKLKHYSCTAEDIDHEDITR |
| | DQTSTLKTCLPLELHKNESCLATRETSSTTRGSCLPPQKTSLMMTLC |
| | LGSIYEDLKMYQTEFQAINAALQNHNHQQIILDKGMLVAIDELMQS |
| | LNHNGETLRQKPPVGEADPYRVKMKLCILLHAFSTRVVTINRVMG |
| | YLSSA (SEQ ID NO: 206) |
| linker | HPVGLLARVPLSYSGHPVGLLARVPLSYSGSGLLSGRSDNHISSGLL |
| | SGRSDNHISSGLLSGRSDNH (SEQ ID NO: 222) |
| Linker | HPVGLLARVPLSYSGHPVGLLARVPLSYSGSGLLSGRSDNHISSGLL |
| | SGRSDNHISSGLLSGRSDNHGGGSGGGS (SEQ ID NO: 232) |
| FNIL12rb1PL1 | QLGASGPGDGCCVEKTSFPEGASGSPLGPRNLSCYRVSKTDYECSW |
| p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSGGGSGGGSPLGVRGKGGGSGG |
| | GSGGGSGGGSGGGSGGGSRVIPVSGPARCLSQSRNLLKTTDDMVK |
| | TAREKLKHYSCTAEDIDHEDITRDQTSTLKTCLPLELHKNESCLATR |
| | ETSSTTRGSCLPPQKTSLMMTLCLGSIYEDLKMYQTEFQAINAALQ |
| | NHNHQQIILDKGMLVAIDELMQSLNHNGETLRQKPPVGEADPYRV |
| | KMKLCILLHAFSTRVVTINRVMGYLSSA (SEQ ID NO: 207) |
| Linker | GGGSGGGSGGGSGGGSPLGVRGKGGGSGGGSGGGSGGGSGGGSG |
| | GGS (SEQ ID NO: 223) |
| FN12mIL12rb1 | QLGASGPGDGCCVEKTSFPEGASGSPLGPRNLSCYRVSKTDYECSW |
| HP3(40)p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSHPVGLLARHPVGLLARHPVGLL |
| | ARGGGSGGGSRVIPVSGPARCLSQSRNLLKTTDDMVKTAREKLKH |
| | YSCTAEDIDHEDITRDQTSTLKTCLPLELHKNESCLATRETSSTTRGS |
| | CLPPQKTSLMMTLCLGSIYEDLKMYQTEFQAINAALQNHNHQQIIL |
| | DKGMLVAIDELMQSLNHNGETLRQKPPVGEADPYRVKMKLCILLH |
| | AFSTRVVTINRVMGYLSSA (SEQ ID NO: 208) |
| Linker | HPVGLLARHPVGLLARHPVGLLAR (SEQ ID NO: 224) |
| Linker | GGGSGGGSHPVGLLARHPVGLLARHPVGLLARGGGSGGGS (SEQ |
| | ID NO: 233) |
| FN12mIL12rb1 | QLGASGPGDGCCVEKTSFPEGASGSPLGPRNLSCYRVSKTDYECSW |
| LS3(49)p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSSGLLSGRSDNHSGLLSGRSDNH |
| | SGLLSGRSDNHGGGSGGGSRVIPVSGPARCLSQSRNLLKTTDDMVK |
| | TAREKLKHYSCTAEDIDHEDITRDQTSTLKTCLPLELHKNESCLATR |
| | ETSSTTRGSCLPPQKTSLMMTLCLGSIYEDLKMYQTEFQAINAALQ |
| | NHNHQQIILDKGMLVAIDELMQSLNHNGETLRQKPPVGEADPYRV |
| | KMKLCILLHAFSTRVVTINRVMGYLSSA (SEQ ID NO: 209) |
| Linker | SGLLSGRSDNHSGLLSGRSDNHSGLLSGRSDNH (SEQ ID NO: 234) |
| Linker | GGGSGGGSSGLLSGRSDNHSGLLSGRSDNHSGLLSGRSDNHGGGS |
| | GGGS (SEQ ID NO: 235) |
| FN12mIL12rb1 | QLGASGPGDGCCVEKTSFPEGASGSPLGPRNLSCYRVSKTDYECSW |
| HPVPLS(43)p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSHPVGLLARVPLSLYSGSGLLSGR |
| | SDNHGGGSGGGSRVIPVSGPARCLSQSRNLLKTTDDMVKTAREKLK |
| | HYSCTAEDIDHEDITRDQTSTLKTCLPLELHKNESCLATRETSSTTRG |
| | SCLPPQKTSLMMTLCLGSIYEDLKMYQTEFQAINAALQNHNHQQII |
| | LDKGMLVAIDELMQSLNHNGETLRQKPPVGEADPYRVKMKLCILL |
| | HAFSTRVVTINRVMGYLSSA (SEQ ID NO: 210) |
| Linker | HPVGLLARVPLSLYSGSGLLSGRSDNH (SEQ ID NO: 236) |
| Linker | GGGSGGGSHPVGLLARVPLSLYSGSGLLSGRSDNHGGGSGGGS |
| | (SEQ ID NO: 237) |
| FN12mIL12rb1 | QLGASGPGDGCCVEKTSFPEGASGSPLGPRNLSCYRVSKTDYECSW |
| HPVP2LS(51) | QYDGPEDNVSHVLWCCFVPPNHTHTGQERCRYFSSGPDRTVQFWE |
| p35 | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAHPVGLLARVPLSLYSGHPVGLLARVPLSLYS |
| | GSGLLSGRSDNHGGGSGGGSRVIPVSGPARCLSQSRNLLKTTDDMV |
| | KTAREKLKHYSCTAEDIDHEDITRDQTSTLKTCLPLELHKNESCLAT |
| | RETSSTTRGSCLPPQKTSLMMTLCLGSIYEDLKMYQTEFQAINAAL |
| | QNHNHQQIILDKGMLVAIDELMQSLNHNGETLRQKPPVGEADPYR |
| | VKMKLCILLHAFSTRVVTINRVMGYLSSA (SEQ ID NO: 211) |
| Linker | HPVGLLARVPLSLYSGHPVGLLARVPLSLYSGSGLLSGRSDNH (SEQ |
| | ID NO: 219) |
| Linker | HPVGLLARVPLSLYSGHPVGLLARVPLSLYSGSGLLSGRSDNHGGG |
| | SGGGS (SEQ ID NO: 220) |
| FN12mIL12rb1 | QLGASGPGDGCCVEKTSFPEGASGSPLGPRNLSCYRVSKTDYECSW |
| VP3(44)p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSVPLSLYSGVPLSLYSGVPLSLYS |
| | GGGGSGGGSGGGSRVIPVSGPARCLSQSRNLLKTTDDMVKTAREKL |
| | KHYSCTAEDIDHEDITRDQTSTLKTCLPLELHKNESCLATRETSSTTR |
| | GSCLPPQKTSLMMTLCLGSIYEDLKMYQTEFQAINAALQNHNHQQI |
| | ILDKGMLVAIDELMQSLNHNGETLRQKPPVGEADPYRVKMKLCILL |
| | HAFSTRVVTINRVMGYLSSA (SEQ ID NO: 212) |
| Linker | VPLSLYSGVPLSLYSGVPLSLYS (SEQ ID NO: 238) |
| Linker | GGGSGGGSVPLSLYSGVPLSLYSGVPLSLYSGGGGSGGGSGGGS |
| | (SEQ ID NO: 239) |
| FN12mIL12rb1 | QLGASGPGDGCCVEKTSFPEGASGSPLGPRNLSCYRVSKTDYECSW |
| VPuPA2(54)p35 | QYDGPEDNVSHVLWCCFVPPNHTHTGQERCRYFSSGPDRTVQFWE |
| | QDGIPVLSKVNFWVESRLGNRTMKSQKISQYLYNWTKTTPPLGHIK |
| | VSQSHRQLRMDWNVSEEAGAEVQFRRRMPTTNWTLGDCGPQVNS |
| | GSGVLGDIRGSMSESCLCPSENMAQEIQIRRRRRLSSGAPGGPWSD |
| | WSMPVCVPPEVLPQAGGGSGGGSVPLSLYSGGGGSGGGSLSGRSD |
| | NHLSGRSDNHGGGSGGGSRVIPVSGPARCLSQSRNLLKTTDDMVK |
| | TAREKLKHYSCTAEDIDHEDITRDQTSTLKTCLPLELHKNESCLATR |
| | ETSSTTRGSCLPPQKTSLMMTLCLGSIYEDLKMYQTEFQAINAALQ |
| | NHNHQQIILDKGMLVAIDELMQSLNHNGETLRQKPPVGEADPYRV |
| | KMKLCILLHAFSTRVVTINRVMGYLSSA (SEQ ID NO: 213) |
| Linker | VPLSLYSGGGGSGGGSLSGRSDNHLSGRSDNH (SEQ ID NO: 240) |
| Linker | GGGSGGGSVPLSLYSGGGGSGGGSLSGRSDNHLSGRSDNHGGGSG |
| | GGS (SEQ ID NO: 241) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| VPuPA2(54) | TPDNQVGAEVQFRHRTPSSPWKLGDCGPQDDDTESCLCPLEMNVA |
| | QEFQLRRRQLGSQGSSWSKWSSPVCVPPEGGGSGGGSVPLSLYSGG |
| | GGSGGGSLSGRSDNHLSGRSDNHGGGSGGGSGRNLPVATPDPGMF |
| | PCLHHSQNLLRAVSNMLQKARQTLEFYPCTSEEIDHEDITKDKTST |
| | VEACLPLELTKNESCLNSRETSFITNGSCLASRKTSFMMALCLSSIYE |
| | DLKMYQVEFKTMNAKLLMDPKRQIFLDQNMLAVIDELMQALNFN |
| | SETVPQKSSLEEPDFYKTKIKLCILLHAFRIRAVTIDRVMSYLNAS |
| | (SEQ ID NO: 214) |
| linker | VPLSLYSGGGGSGGGSLSGRSDNHLSGRSDNH (SEQ ID NO: 240) |
| Linker | GGGSGGGSVPLSLYSGGGGSGGGSLSGRSDNHLSGRSDNHGGGSG |
| | GGSG (SEQ ID NO: 242) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| VP3(44) | TPDNQVGAEVQFRHRTPSSPWKLGDCGPQDDDTESCLCPLEMNVA |
| | QEFQLRRRQLGSQGSSWSKWSSPVCVPPEGGGSGGGSVPLSLYSGV |
| | PLSLYSGVPLSLYSGGGGSGGGSGGGSRNLPVATPDPGMFPCLHHS |
| | QNLLRAVSNMLQKARQTLEFYPCTSEEIDHEDITKDKTSTVEACLPL |
| | ELTKNESCLNSRETSFITNGSCLASRKTSFMMALCLSSIYEDLKMYQ |
| | VEFKTMNAKLLMDPKRQIFLDQNMLAVIDELMQALNFNSETVPQK |
| | SSLEEPDFYKTKIKLCILLHAFRIRAVTIDRVMSYLNAS (SEQ ID |
| | NO: 215) |
| linker | VPLSLYSGVPLSLYSGVPLSLYS (SEQ ID NO: 238) |
| Linker | GGGSGGGSVPLSLYSGVPLSLYSGVPLSLYSGGGGSGGGSGGGS |
| | (SEQ ID NO: 239) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| HPVPLS(43) | TPDNQVGAEVQFRHRTPSSPWKLGDCGPQDDDTESCLCPLEMNVA |
| | QEFQLRRRQLGSQGSSWSKWSSPVCVPPEGGGSGGGSHPVGLLAR |
| | VPLSLYSGSGLLSGRSDNHGGGSGGGSRNLPVATPDPGMFPCLHHS |
| | QNLLRAVSNMLQKARQTLEFYPCTSEEIDHEDITKDKTSTVEACLPL |
| | ELTKNESCLNSRETSFITNGSCLASRKTSFMMALCLSSIYEDLKMYQ |
| | VEFKTMNAKLLMDPKRQIFLDQNMLAVIDELMQALNFNSETVPQK |
| | SSLEEPDFYKTKIKLCILLHAFRIRAVTIDRVMSYLNAS (SEQ ID |
| | NO: 216) |
| linker | HPVGLLARVPLSLYSGSGLLSGRSDNH (SEQ ID NO: 236) |
| Linker | GGGSGGGSHPVGLLARVPLSLYSGSGLLSGRSDNHGGGSGGGS |
| | (SEQ ID NO: 237) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| PLGER2 | TPDNQVGAEVQFRHRTPSSPWKLGDCGPQDDDTESCLCPLEMNVA |
| | QEFQLRRRQLGSQGSSWSKWSSPVCVPPEPLGVRGKPLGVRGKGE |
| | RGPPGPQGAARGFPGTPGLGERGPPGPQGAARGFPGTPGLGGGSG |
| | GGSRNLPVATPDPGMFPCLHHSQNLLRAVSNMLQKARQTLEFYPCT |
| | SEEIDHEDITKDKTSTVEACLPLELTKNESCLNSRETSFITNGSCLAS |
| | RKTSFMMALCLSSIYEDLKMYQVEFKTMNAKLLMDPKRQIFLDQN |
| | MLAVIDELMQALNFNSETVPQKSSLEEPDFYKTKIKLCILLHAFRIR |
| | AVTIDRVMSYLNAS (SEQ ID NO: 217) |
| linker | PLGVRGKPLGVRGKGERGPPGPQGAARGFPGTPGLGERGPPGPQG |
| | AARGFPGTPGL (SEQ ID NO: 243) |
| Linker | PLGVRGKPLGVRGKGERGPPGPQGAARGFPGTPGLGERGPPGPQG |
| | AARGFPGTPGLGGGSGGGS (SEQ ID NO: 244) |
| Human FNI-II | CRTSECCFQDPPYPDADSGSASGPRDLRCYRISSDRYECSWQYEGP |
| domain IL- | TAGVSHFLRCCLSSGRCCYFAAGSATRLQFSDQAGVSVLYTVTLWV |
| 12Rß1-Hu_p35 | ESWARNQTEKSPEVTLQLYNSVKYEPPLGDIKVSKLAGQLRMEWE |
| HPVPLSPL2 | TPDNQVGAEVQFRHRTPSSPWKLGDCGPQDDDTESCLCPLEMNVA |
| | QEFQLRRRQLGSQGSSWSKWSSPVCVPPEHPVGLLARVPLSYSGHP |
| | VGLLARVPLSYSGSGLLSGRSDNHPLGVRGKPLGVRGKGGGSGGG |
| | SRNLPVATPDPGMFPCLHHSQNLLRAVSNMLQKARQTLEFYPCTSE |
| | EIDHEDITKDKTSTVEACLPLELTKNESCLNSRETSFITNGSCLASRK |
| | TSFMMALCLSSIYEDLKMYQVEFKTMNAKLLMDPKRQIFLDQNML |
| | AVIDELMQALNFNSETVPQKSSLEEPDFYKTKIKLCILLHAFRIRAVT |
| | IDRVMSYLNAS (SEQ ID NO: 218) |
| Linker | HPVGLLARVPLSYSGHPVGLLARVPLSYSGSGLLSGRSDNHPLGVR |
| | GKPLGVRGK (SEQ ID NO: 245) |
| Linker | HPVGLLARVPLSYSGHPVGLLARVPLSYSGSGLLSGRSDNHPLGVR |
| | GKPLGVRGKGGGSGGGS (SEQ ID NO: 246) |
| Mouse M- | QLGASGPGDCCVEKTSFPEGASGSPLGPRNLSCYRVSKTDYECSWQ |
| (G₃S)₁₁-p35 | YDGPEDNVSHVLWCCFVPPNHTHTGQERCRYFSSGPDRTVQFWEQ |
| | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQAGGGSGGGSGGGSGGGSGGGSGGGSGGGSGG |
| | GSGGGSGGGSGGGSRVIPVSGPARCLSQSRNLLKTTDDMVKTAREK |
| | LKHYSCTAEDIDHEDITRDQTSTLKTCLPLELHKNESCLATRETSSTT |
| | RGSCLPPQKTSLMMTLCLGSIYEDLKMYQTEFQAINAALQNHNHQ |
| | QIILDKGMLVAIDELMQSLNHNGETLRQKPPVGEADPYRVKMKLCI |
| | LLHAFSTRVVTINRVMGYLSSAHHHHHH (SEQ ID NO: 247) |
| IL-12 Mask | QLGASGPGDCCVEKTSFPEGASGSPLGPRNLSCYRVSKTDYECSWQ |
| | YDGPEDNVSHVLWCCFVPPNHTHTGQERCRYFSSGPDRTVQFWEQ |
| | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDW |
| | SMPVCVPPEVLPQA (SEQ ID NO: 192) |
| linker | GGGSGGGSGGGSGGGSGGGSGGGSGGGSGGGSGGGSGGGSGGGS |
| | (SEQ ID NO: 48) |
| p35 | RVIPVSGPARCLSQSRNLLKTTDDMVKTAREKLKHYSCTAEDIDHE |
| | DITRDQTSTLKTCLPLELHKNESCLATRETSSTTRGSCLPPQKTSLM |
| | MTLCLGSIYEDLKMYQTEFQAINAALQNHNHQQIILDKGMLVAIDE |
| | LMQSLNHNGETLRQKPPVGEADPYRVKMKLCILLHAFSTRVVTINR |
| | VMGYLSSA (SEQ ID NO: 5) |
| Mouse p40 | MWELEKDVYVVEVDWTPDAPGETVNLTCDTPEEDDITWTSDQRH |
| | GVIGSGKTLTITVKEFLDAGQYTCHKGGETLSHSHLLLHKKENGIW |
| | STEILKNFKNKTFLKCEAPNYSGRFTCSWLVQRNMDLKFNIKSSSSS |
| | PDSRAVTCGMASLSAEKVTLDQRDYEKYSVSCQEDVTCPTAEETLP |
| | IELALEARQQNKYENYSTSFFIRDIIKPDPPKNLQMKPLKNSQVEVS |
| | WEYPDSWSTPHSYFSLKFFVRIQRKKEKMKETEEGCNQKGAFLVE |
| | KTSTEVQCKGGNVCVQAQDRYYNSSCSKWACVPCRVRS (SEQ ID |
| | NO: 6) |
| Human M- | CRTSECCFQDPPYPDADSGSASGPRDLRCYRISSDRYECSWQYEGP |
| (G₃S)₁₁-p35 | TAGVSHFLRCCLSSGRCCYFAAGSATRLQFSDQAGVSVLYTVTLWV |
| | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPEGGGSGGGSGGGSGGGS |
| | GGGSGGGSGGGSGGGSGGGSGGGSGGGSGRNLPVATPDPGMFPCL |
| | HHSQNLLRAVSNMLQKARQTLEFYPCTSEEIDHEDITKDKTSTVEA |
| | CLPLELTKNESCLNSRETSFITNGSCLASRKTSFMMALCLSSIYEDLK |
| | MYQVEFKTMNAKLLMDPKRQIFLDQNMLAVIDELMQALNFNSET |
| | VPQKSSLEEPDFYKTKIKLCILLHAFRIRAVTIDRVMSYLNASHHHH |
| | HH (SEQ ID NO: 248) |
| IL-12 Mask | CRTSECCFQDPPYPDADSGSASGPRDLRCYRISSDRYECSWQYEGP |
| | TAGVSHFLRCCLSSGRCCYFAAGSATRLQFSDQAGVSVLYTVTLWV |
| | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPE (SEQ ID NO: 195) |
| Linker | GGGSGGGSGGGSGGGSGGGSGGGSGGGSGGGSGGGSGGGSGGGS |
| | (SEQ ID NO: 48) |
| p35 | RNLPVATPDPGMFPCLHHSQNLLRAVSNMLQKARQTLEFYPCTSEEI |
| | DHEDITKDKTSTVEACLPLELTKNESCLNSRETSFITNGSCLASRKTS |
| | FMMALCLSSIYEDLKMYQVEFKTMNAKLLMDPKRQIFLDQNMLA |
| | VIDELMQALNFNSETVPQKSSLEEPDFYKTKIKLCILLHAFRIRAVTI |
| | DRVMSYLNAS (SEQ ID NO: 3) |
| Human p40 | IWELKKDVYVVELDWYPDAPGEMVVLTCDTPEEDGITWTLDQSSE |
| | VLGSGKTLTIQVKEFGDAGQYTCHKGGEVLSHSLLLLHKKEDGIWS |
| | TDILKDQKEPKNKTFLRCEAKNYSGRFTCWWLTTISTDLTFSVKSSR |
| | GSSDPQGVTCGAATLSAERVRGDNKEYEYSVECQEDSACPAAEESL |
| | PIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVI |
| | CRKNASISVRAQDRYYSSSWSEWASVPCS (SEQ ID NO: 4) |

In some aspects, the polypeptide has at least 75, 76, 77, 78, 79, 80, 81, 82, 83, 84, 85, 86, 87, 88, 89, 90, 91, 92, 93, 94, 95, 96, 97, 98, 99, or 100% identity (or any derivable range therein) to a polypeptide of the disclosure, such as SEQ ID NOS:7-12, and 15-18, or fragments thereof.

### F. Protein Tags

In some aspects, the polypeptide further comprises a protein tag. The protein tag can be used for protein purification and/or immunoassays, for example. Exemplary protein tags include AviTag, a peptide allowing biotinylation by the enzyme BirA and so the protein can be isolated by streptavidin (GLNDIFEAQKIEWHE (SEQ ID NO:82)), Calmodulin-tag, a peptide bound by the protein calmodulin (KRRWKKNFIAVSAANRFKKISSSGAL (SEQ ID NO:83)), polyglutamate tag, a peptide binding efficiently to anion-exchange resin such as Mono-Q (EEEEEE (SEQ ID NO:84)), E-tag, a peptide recognized by an antibody (GAPVPYPDPLEPR (SEQ ID NO:102)), FLAG-tag, a peptide recognized by an antibody (DYKDDDDK (SEQ ID NO:85)), HA-tag, a peptide from hemagglutinin recognized by an antibody (YPYDVPDYA (SEQ ID NO:86)), His-tag, 5-10 histidines (HHHHH—SEQ ID NO:193) bound by a nickel or cobalt chelate (HHHHHH (SEQ ID NO:87)), Myc-tag, a peptide derived from c-myc recognized by an antibody (EQKLISEEDL (SEQ ID NO:88)), NE-tag, a novel 18-amino-acid synthetic peptide (TKENPRSNQEESYDDNES (SEQ ID NO:89)) recognized by a monoclonal IgG1 antibody, which is useful in a wide spectrum of applications including Western blotting, ELISA, flow cytometry, immunocytochemistry, immunoprecipitation, and affinity purification of recombinant proteins, S-tag, a peptide derived from Ribonuclease A (KETAAAKFERQHMDS (SEQ ID NO:90)), SBP-tag, a peptide which binds to streptavidin (MDEKTTGWRGGHVVEGLAGELEQLRARLEHHPQGQREP (SEQ ID NO:91)), Softag 1, for mammalian expression (SLAELLNAGLGGS (SEQ ID NO:92)), Softag 3, for prokaryotic expression (TQDPSRVG (SEQ ID NO:93)), Strep-tag, a peptide which binds to streptavidin or the modified streptavidin called streptactin (Strep-tag II: WSHPQFEK (SEQ ID NO:94)), TC tag, a tetracysteine tag that is recognized by FLASH and ReAsH biarsenical compounds (CCPGCC (SEQ ID NO:95)), V5 tag, a peptide recognized by an antibody (GKPIPNPLLGLDST (SEQ ID NO:96)), VSV-tag, a peptide recognized by an antibody (YTDIEMNRLGK (SEQ ID NO:97)), Xpress tag (DLYDDDDK (SEQ ID NO:98)), Covalent peptide tags, Isopeptag, a peptide which binds covalently to pilin-C protein (TDKDMTITFTNKKDAE (SEQ ID NO:99)), SpyTag, a peptide which binds covalently to SpyCatcher protein (AHIVMVDAYKPTK (SEQ ID NO:100)), SnoopTag, a peptide which binds covalently to SnoopCatcher protein (KLGDIEFIKVNK (SEQ ID NO:101)), BCCP (Biotin Carboxyl Carrier Protein), a protein domain biotinylated by BirA enabling recognition by streptavidin, Glutathione-S-transferase-tag, a protein which binds to immobilized glutathione, Green fluorescent protein-tag, a protein which is spontaneously fluorescent and can be bound by nanobodies, HaloTag, a mutated bacterial haloalkane dehalogenase that covalently attaches to a reactive haloalkane substrate, this allows attachment to a wide variety of substrates. Maltose binding protein-tag, a protein which binds to amylose agarose, Nus-tag, Thioredoxin-tag, Fc-tag, derived from immunoglobulin Fc domain, allow dimerization and solubilization. Can be used for purification on Protein-A Sepharose, Designed Intrinsically Disordered tags containing disorder promoting amino acids (P,E,S,T,A,Q,G,), and Ty-tag. In some aspects, the polypeptide comprises a 6H tag of SEQ ID NO:87.

### II. PROTEINACEOUS COMPOSITIONS

The polypeptides or polynucleotides of the disclosure, such as the CBD, serum protein, therapeutic agent, masking agent, linker, or cytokine polypeptide, may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleic acids, or any range derivable therein, of SEQ ID NOS: 1-249.

The polypeptides of the disclosure, such as the CBD, serum protein, therapeutic agent, masking agent, linker, or cytokine polypeptide, may include 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids, or any range derivable therein, of SEQ ID NOS: 1-249.

In some aspects, a polypeptide of the disclosure may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) of SEQ ID NOS: 1-249.

In some aspects, a polypeptide of the disclosure, such as the CBD, serum protein, therapeutic agent, masking agent, linker, or cytokine polypeptide, may comprise at least, at most, about, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOS: 1-249.

In some aspects, the polypeptide, such as the CBD, serum protein, therapeutic agent, masking agent, linker, or cytokine polypeptide, may comprise at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 (or any derivable range therein) contiguous amino acids of SEQ ID NOS:1-249 that are at least, at most, or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with one of SEQ ID NOS: 1-249.

A polypeptide of the disclosure, such as the CBD, serum protein, therapeutic agent, masking agent, linker, or cytokine polypeptide may be at least, at most, or about 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any range derivable therein) similar, identical, or homologous with one of SEQ ID NOS: 1-249.

The polypeptides and nucleic acids of the disclosure may include at least, at most, about, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 of one of SEQ ID NOS: 1-249. One or more of these substitutions may be specifically excluded from an aspect.

Peptides, polypeptides, and proteins of the disclosure, such as the CBD, serum protein, therapeutic agent, masking agent, linker, or cytokine polypeptide, having at least, having at least, or having 70, 75, 80, 85, 90, 91, 92, 93, 94, 95, 96, 97, 98, 99, 100% identity to any one of SEQ ID NOS: 1-249 includes a fragment of segment starting at amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any range derivable therein) and ending at amino acid 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, or 205 (or any range derivable therein).

In some aspects, the amino acid at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, or 400 of the peptide or polypeptide of one of SEQ ID NOS:1-249 is substituted with an alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, or valine.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa. One or more of these substitutions may be specifically excluded from an aspect.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other aspects, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific aspects, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One aspect includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. NUCLEIC ACIDS

In certain aspects, the current disclosure concerns recombinant polynucleotides encoding the proteins, polypeptides, and peptides of the invention, such as the CBD, serum protein, therapeutic agent, masking agent, linker, or cytokine polypeptide, and/or other molecules. Therefore, certain aspects relate to nucleotides encoding for a CBD, serum protein, therapeutic agent, masking agent, linker, or cytokine polypeptide, and fragments thereof.

As used in this application, the term “polynucleotide” refers to a nucleic acid molecule that either is recombinant or has been isolated free of total genomic nucleic acid. Included within the term “polynucleotide” are oligonucleotides (nucleic acids of 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence of: 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1095, 1100, 1500, 2000, 2500, 3000, 3500, 4000, 4500, 5000, 5500, 6000, 6500, 7000, 7500, 8000, 9000, 10000, or more nucleotides, nucleosides, or base pairs (or any range derivable therein), including all values and ranges there between, of a polynucleotide encoding one or more amino acid sequence described or referenced herein. It also is contemplated that a particular polypeptide may be encoded by nucleic acids containing variations having slightly different nucleic acid sequences but, nonetheless, encode the same or substantially similar protein.

In particular aspects, the invention concerns isolated nucleic acid segments and recombinant vectors incorporating nucleic acid sequences that encode a polypeptide or peptide of the disclosure. The term “recombinant” may be used in conjunction with a polynucleotide or polypeptide and generally refers to a polypeptide or polynucleotide produced and/or manipulated in vitro or that is a replication product of such a molecule.

In other aspects, the invention concerns isolated nucleic acid segments and recombinant vectors incorporating nucleic acid sequences that encode a polypeptide or peptide of the disclosure.

The nucleic acid segments used in the current disclosure can be combined with other nucleic acid sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

In certain aspects, the current disclosure provides polynucleotide variants having substantial identity to the sequences disclosed herein; those comprising at least 70%, 75%, 80%, 85%, 90%, 95%, 96%, 97%, 98%, or 99% or higher sequence identity, including all values and ranges there between, compared to a polynucleotide sequence of this disclosure using the methods described herein (e.g., BLAST analysis using standard parameters).

The disclosure also contemplates the use of polynucleotides which are complementary to all the above described polynucleotides.

### A. Vectors

Polypeptides of the disclosure may be encoded by a nucleic acid molecule comprised in a vector. The term “vector” is used to refer to a carrier nucleic acid molecule into which a heterologous nucleic acid sequence can be inserted for introduction into a cell where it can be replicated and expressed. A nucleic acid sequence can be “heterologous,” which means that it is in a context foreign to the cell in which the vector is being introduced or to the nucleic acid in which is incorporated, which includes a sequence homologous to a sequence in the cell or nucleic acid but in a position within the host cell or nucleic acid where it is ordinarily not found. Vectors include DNAs, RNAs, plasmids, cosmids, viruses (bacteriophage, animal viruses, and plant viruses), and artificial chromosomes (e.g., YACs). One of skill in the art would be well equipped to construct a vector through standard recombinant techniques (for example Sambrook et al., 2001; Ausubel et al., 1996, both incorporated herein by reference). In addition to encoding a polypeptide of the disclosure, the vector can encode other polypeptide sequences such as a one or more other bacterial peptide, a tag, or an immunogenicity enhancing peptide. Useful vectors encoding such fusion proteins include pIN vectors (Inouye et al., 1985), vectors encoding a stretch of histidines, and pGEX vectors, for use in generating glutathione S-transferase (GST) soluble fusion proteins for later purification and separation or cleavage.

The term “expression vector” refers to a vector containing a nucleic acid sequence coding for at least part of a gene product capable of being transcribed. In some cases, RNA molecules are then translated into a protein, polypeptide, or peptide. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described herein.

### B. Promoters and Enhancers

A “promoter” is a control sequence. The promoter is typically a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and expression of that sequence. A promoter may or may not be used in conjunction with an “enhancer,” which refers to a cis-acting regulatory sequence involved in the transcriptional activation of a nucleic acid sequence.

Naturally, it may be important to employ a promoter and/or enhancer that effectively directs the expression of the DNA segment in the cell type or organism chosen for expression. Those of skill in the art of molecular biology generally know the use of promoters, enhancers, and cell type combinations for protein expression (see Sambrook et al., 2001, incorporated herein by reference). The promoters employed may be constitutive, tissue-specific, or inducible and in certain aspects may direct high level expression of the introduced DNA segment under specified conditions, such as large-scale production of recombinant proteins or peptides.

The particular promoter that is employed to control the expression of peptide or protein encoding polynucleotide of the invention is not believed to be critical, so long as it is capable of expressing the polynucleotide in a targeted cell, preferably a bacterial cell. Where a human cell is targeted, it is preferable to position the polynucleotide coding region adjacent to and under the control of a promoter that is capable of being expressed in a human cell. Generally speaking, such a promoter might include either a bacterial, human or viral promoter.

### C. Initiation Signals and Internal Ribosome Binding Sites (IRES)

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals.

In certain aspects of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988; Macejak and Sarnow, 1991). IRES elements can be linked to heterologous open reading frames. Multiple open reading frames can be transcribed together, each separated by an IRES, creating polycistronic messages. Multiple genes can be efficiently expressed using a single promoter/enhancer to transcribe a single message (see U.S. Pat. Nos. 5,925,565 and 5,935,819, herein incorporated by reference).

### D. Selectable and Screenable Markers

In certain aspects of the invention, cells containing a nucleic acid construct of the current disclosure may be identified in vitro or in vivo by encoding a screenable or selectable marker in the expression vector. When transcribed and translated, a marker confers an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

### E. Host Cells

As used herein, the terms “cell,” “cell line,” and “cell culture” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organism that is capable of replicating a vector or expressing a heterologous gene encoded by a vector. A host cell can, and has been, used as a recipient for vectors or viruses. A host cell may be “transfected” or “transformed,” which refers to a process by which exogenous nucleic acid, such as a recombinant protein-encoding sequence, is transferred or introduced into the host cell. A transformed cell includes the primary subject cell and its progeny.

Host cells may be derived from prokaryotes or eukaryotes, including bacteria, yeast cells, insect cells, and mammalian cells for replication of the vector or expression of part or all of the nucleic acid sequence(s). Numerous cell lines and cultures are available for use as a host cell, and they can be obtained through the American Type Culture Collection (ATCC), which is an organization that serves as an archive for living cultures and genetic materials (www.atcc.org).

### F. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. Nos. 5,871,986, 4,879,236, both herein incorporated by reference, and which can be bought, for example, under the name MAXBAC® 2.0 from INVITROGEN® and BACPACK™ BACULOVIRUS EXPRESSION SYSTEM FROM CLONTECH®.

In addition to the disclosed expression systems of the invention, other examples of expression systems include STRATAGENE®'s COMPLETE CONTROL Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

### IV. ADDITIONAL THERAPIES

### A. Immunotherapy

In some aspects, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

### 3. Checkpoint Inhibitors and Combination Treatment

Aspects of the disclosure may include administration of immune checkpoint inhibitors (also referred to as checkpoint inhibitor therapy), which are further described below.

b. PD-1, PD-L1, and PD-L2 inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some aspects, PD-1, PD-L1, and PD-L2 are human PD-1, PD-L1 and PD-L2.

In some aspects, the PD-1 inhibitor is a molecule that inhibits the binding of PD-1 to its ligand binding partners. In a specific aspect, the PD-1 ligand binding partners are PD-L1 and/or PD-L₂. In another aspect, a PD-L1 inhibitor is a molecule that inhibits the binding of PD-L1 to its binding partners. In a specific aspect, PD-L1 binding partners are PD-1 and/or B7-1. In another aspect, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some aspects, the PD-1 inhibitor is an anti-PD-1 antibody (e.g., a human antibody, a humanized antibody, or a chimeric antibody). In some aspects, the anti-PD-1 antibody is selected from the group consisting of nivolumab, pembrolizumab, and pidilizumab. In some aspects, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some aspects, the PD-L1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDI0680, also known as AMP-514, and REGN2810.

In some aspects, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some aspects, the inhibitor comprises the heavy and light chain CDRs or VRs of nivolumab, pembrolizumab, or pidilizumab. Accordingly, in one aspect, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another aspect, the antibody competes for binding with and/or binds to the same epitope on PD-1, PD-L1, or PD-L2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

c. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some aspects, the inhibitor blocks the CTLA-4 and B7-1 interaction. In some aspects, the inhibitor blocks the CTLA-4 and B7-2 interaction.

In some aspects, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO0 1/14424).

In some aspects, the inhibitor comprises the heavy and light chain CDRs or VRs of tremelimumab or ipilimumab. Accordingly, in one aspect, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another aspect, the antibody competes for binding with and/or binds to the same epitope on PD-1, B7-1, or B7-2 as the above-mentioned antibodies. In another aspect, the antibody has at least about 70, 75, 80, 85, 90, 95, 97, or 99% (or any derivable range therein) variable region amino acid sequence identity with the above-mentioned antibodies.

### 4. Inhibition of Co-Stimulatory Molecules

In some aspects, the immunotherapy comprises an inhibitor of a co-stimulatory molecule. In some aspects, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

### 5. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

### 6. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signalling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some aspects, the CAR-T therapy targets CD19.

### 7. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 8. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, aspects of the disclosure include patients that have been previously treated for a therapy described herein, are currently being treated for a therapy described herein, or have not been treated for a therapy described herein. In some aspects, the patient is one that has been determined to be resistant to a therapy described herein. In some aspects, the patient is one that has been determined to be sensitive to a therapy described herein.

### B. Oncolytic Virus

In some aspects, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

### C. Polysaccharides

In some aspects, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

### D. Neoantigens

In some aspects, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

### E. Chemotherapies

In some aspects, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some aspects, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain aspects. In some aspects, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain aspects, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable aspect, the chemotherapeutic agent may be administered in an amount effective to cause arrest or regression of the cancer in a host, when the chemotherapy is administered with the construct. In other aspects, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### F. Radiotherapy

In some aspects, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some aspects, the amount of ionizing radiation is greater than 20 Gy and is administered in one dose. In some aspects, the amount of ionizing radiation is 18 Gy and is administered in three doses. In some aspects, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some aspects, the ionizing radiation is administered in at least, at most, or exactly 1, 2, 3, 4, 5, 6, 7, 8, 9, or 10 doses (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some aspects, the amount of IR may be presented as a total dose of IR, which is then administered in fractionated doses. For example, in some aspects, the total dose is 50 Gy administered in 10 fractionated doses of 5 Gy each. In some aspects, the total dose is 50-90 Gy, administered in 20-60 fractionated doses of 2-3 Gy each. In some aspects, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some aspects, the total dose is administered in fractionated doses of at least, at most, or about 1, 2, 3, 4, 5, 6, 7, 8, 9, 10, 12, 14, 15, 20, 25, 30, 35, 40, 45, or 50 Gy (or any derivable range therein. In some aspects, at least, at most, or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some aspects, at least, at most, or about 1, 2, 3, 4, 5, 6, 7, 8, 9, 10, 11, or 12 (or any derivable range therein) fractionated doses are administered per day. In some aspects, at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present aspects, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### H. Other Agents

It is contemplated that other agents may be used in combination with certain aspects of the present aspects to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other aspects, cytostatic or differentiation agents can be used in combination with certain aspects of the present aspects to improve the anti-hyperproliferative efficacy of the treatments. Inhibitors of cell adhesion are contemplated to improve the efficacy of the present aspects. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present aspects to improve the treatment efficacy.

### V. COMBINATION THERAPY

The compositions and related methods of the present disclosure, particularly administration of the masked therapeutic agents of the disclosure may also be used in combination with the administration of additional therapies such as the additional therapeutics described herein or in combination with other traditional therapeutics known in the art for the treatment of cancer.

The therapeutic compositions and treatments disclosed herein may precede, be co-current with and/or follow another treatment or agent by intervals ranging from minutes to weeks. In aspects where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

Various combination regimens of the therapeutic agents and treatments may be employed. Non-limiting examples of such combinations are shown below, wherein a therapeutic agent such as a composition disclosed herein is “A” and a second agent, such as an additional agent or therapy described herein or known in the art is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

In some aspects, more than one course of therapy may be employed. It is contemplated that multiple courses may be implemented.

### VI. THERAPEUTIC METHODS

The current methods and compositions relate to methods for treating cancer. In some aspects, the cancer comprises a solid tumor. In some aspects, the cancer is non-lymphatic. In some aspects, the cancer is breast cancer or colon cancer.

The compositions of the disclosure may be used for in vivo, in vitro, or ex vivo administration. The route of administration of the composition may be, for example, intratumoral, intracutaneous, subcutaneous, intravenous, intralymphatic, and intraperitoneal administrations. In some aspects, the administration is intratumoral or intralymphatic or peri-tumoral. In some aspects, the compositions are administered directly into a cancer tissue or a lymph node.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

The cancers amenable for treatment include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VII. PHARMACEUTICAL COMPOSITIONS AND METHODS

In some aspects, pharmaceutical compositions are administered to a subject. Different aspects involve administering an effective amount of a composition to a subject. In some aspects, a composition comprising an inhibitor may be administered to the subject or patient to treat cancer or reduce the size of a tumor. Additionally, such compounds can be administered in combination with an additional cancer therapy.

Compositions can be formulated for parenteral administration, e.g., formulated for injection via the intravenous, transcatheter injection, intraarterial injection, intramuscular, subcutaneous, or even intraperitoneal routes. Typically, such compositions can be prepared as injectables, either as liquid solutions or suspensions; solid forms suitable for use to prepare solutions or suspensions upon the addition of a liquid prior to injection can also be prepared; and, the preparations can also be emulsified. The preparation of such formulations will be known to those of skill in the art in light of the present disclosure. Other routes of administration include intratumoral, peri-tumoral, intralymphatic, injection into cancer tissue, and injection into lymph nodes. In some aspects, the administration is systemic.

The pharmaceutical forms suitable for injectable use include sterile aqueous solutions or dispersions; formulations including sesame oil, peanut oil, or aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The carrier also can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

As used herein, the term “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable carrier,” means a pharmaceutically acceptable material, composition or vehicle, such as a liquid or solid filler, diluent, excipient, solvent or encapsulating material, involved in carrying or transporting a chemical agent.

As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. Pharmaceutically acceptable salts include the conventional non-toxic salts or the quaternary ammonium salts of the parent compound formed, for example, from non-toxic inorganic or organic acids. The pharmaceutically acceptable salts can be synthesized from the parent compound which contains a basic or acidic moiety by conventional chemical methods.

Some variation in dosage will necessarily occur depending on the condition of the subject. The person responsible for administration will, in any event, determine the appropriate dose for the individual subject. An effective amount of therapeutic or prophylactic composition is determined based on the intended goal. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the effects desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

Upon formulation, solutions will be administered in a manner compatible with the dosage formulation and in such amount as is therapeutically or prophylactically effective. The formulations are easily administered in a variety of dosage forms, such as the type of injectable solutions described above.

Typically, for a human adult (weighing approximately 70 kilograms), from about 0.1 mg to about 3000 mg (including all values and ranges there between), or from about 5 mg to about 1000 mg (including all values and ranges there between), or from about 10 mg to about 100 mg (including all values and ranges there between), of a compound are administered. It is understood that these dosage ranges are by way of example only, and that administration can be adjusted depending on the factors known to the skilled artisan.

In certain aspects, a subject is administered about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

A dose may be administered on an as needed basis or every 1, 2, 3, 4, 5, 6, 7, 8, 9, 10, 11, 12, 18, or 24 hours (or any range derivable therein) or 1, 2, 3, 4, 5, 6, 7, 8, 9, or times per day (or any range derivable therein). A dose may be first administered before or after signs of a condition. In some aspects, the patient is administered a first dose of a regimen 1, 2, 3, 4, 5, 6, 7, 8, 9, 10, 11, 12 hours (or any range derivable therein) or 1, 2, 3, 4, or 5 days after the patient experiences or exhibits signs or symptoms of the condition (or any range derivable therein). The patient may be treated for 1, 2, 3, 4, 5, 6, 7, 8, 9, 10 or more days (or any range derivable therein) or until symptoms of the condition have disappeared or been reduced or after 6, 12, 18, or 24 hours or 1, 2, 3, 4, or 5 days after symptoms of an infection have disappeared or been reduced.

### VIII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Fusing Cytokine Receptor to Cytokine as a Mask of Cytokine Activity to Decrease its Toxicity and Increase its Tumor Specific Activation

According to the National Cancer Institute, cancer is the second leading cause of death in the US. Cancer immunotherapy has revolutionized the way oncology is practiced clinically, thanks to checkpoint-blocking antibodies (for solid tumors) and adoptive T cell therapy (for liquid cancers). In patients with solid malignancies, checkpoint inhibitor (CPI) therapy induces durable responses in a subset (12.46% as of 2018) of patients (1). Much work has been and continues to be done to understand why only a fraction of patients respond to CPI therapy (2). These studies indicate that the success of CPI therapy depends on pre-existing intratumoral inflammation (3), particularly baseline evidence of activated T cells (4), IFNg-related gene expression signature (5,6) and expression of major histocompatibility complex (MHC) molecules (7). In order to further increase the response rates, novel treatment strategies that focus on inducing inflammation need to be brought to the clinic. Proinflammatory cytokines are powerful molecules capable of driving the differentiation of tumor-reactive CD8+ T cells, activating IFNg pathway and increasing the expression of MHC class I and II molecules. Despite this, their use in the clinic has been limited due to dose-limiting immune-related adverse events (irAEs) caused by these cytokines. Overcoming this challenge is an important step towards the successful translation of cytokines and their broad use in the clinic, as well as design of novel combination immunotherapy strategies.

IL-12 is a 60 kDa heterodimeric cytokine, composed of p35 and p40 subunits connected via a disulfide bond. It binds to the IL-12 receptor complex consisting of IL-12Rb1 and IL-12Rb2, induces phosphorylation of signal transducer and activator of transcription 4 (STAT4) and results in IFNg secretion by T and natural killer (NK) cells. IL-12 plays a crucial role in linking the innate and adaptive arms of immunity(8), as it activates CD4/8 T cells and drives T helper 1-(Th1)-biased response (9), acts as a growth factor for NK cells (10), and primes myeloid cells by augmenting MHC expression (11,12). IFNg itself can act directly on tumor cells, as IFNg receptor complex (IFNGR1 and IFNGR2) is expressed by cancer cells. IFNg exerts potent anti-angiogenic, anti-proliferative and pro-apoptotic effects on cancer cells (13). Most of antitumor activities of IL-12 are associated with IFNg (14), and so are the irAEs (15). In clinical trials, the main toxicities of IL-12 and IFNg therapies were “flu-like” symptoms (16,17) and elevations of liver enzymes (18). Thus, a successful IL-12 therapy must minimize levels of circulating IFNg, while maintaining its level in the tumor (19).

Type I IFNs (IFNa/b) were discovered as potent antiviral cytokines (20) and are now widely acknowledged as critical components of antitumor immunity as well (21). There are many type I IFN members and they all bind to a type I IFN-specific heterodimeric receptor complex, IFNAR1 and IFNAR2. Although IFNa/b and IFNg structurally resemble each other and elicit partially overlapping gene signature, responses stimulated by type I IFNs seem to be broader (22). Type I IFNs activate mainly STAT1, STAT2, but can also activate STAT3 and STAT5 (23-25), whereas IFNg primarily phosphorylates STAT1. IFNa can induce apoptosis of cancer cells, maturation of dendritic cells (DCs) and production of IL-15 by DCs (26). IFNa is approved for the treatment of hairy cell leukemia (27), Kaposi's sarcoma in patients with acquired immunodeficiency syndrome (28) and advanced melanoma (21), yet dose-limiting irAEs occur in 30-50% of the patients (29). In order to extend its serum half-life and decrease the number of doses, polyethylene glycol (PEG) was conjugated to IFNa-2b (PEG Intron) (30). Although this strategy provided a more serum-persistent form of IFNa, severe side effects were still reported, perhaps due to binding to IFNAR expressed on circulating cells and in healthy tissues (31). Engineering type I IFNs that can be activated specifically in the tumor would mitigate the irAEs and increase the fraction of patients benefiting from the therapy.

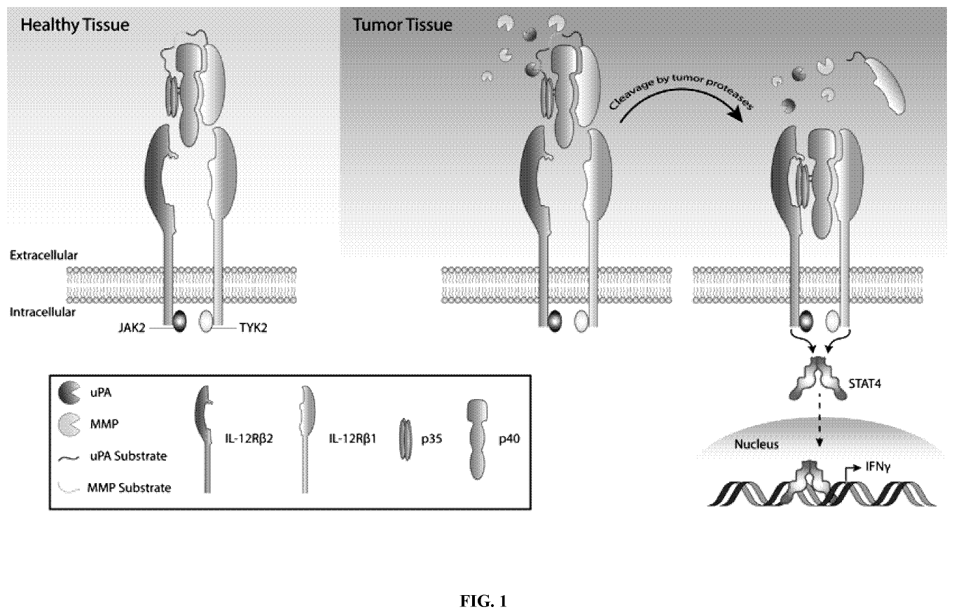

Here, the inventors sought to develop binding site masking methodology to IL-12 and IFNs, to improve its toxicity. They have used IL-12 receptor beta 1 (IL-12Rb1) fibronectin I and II domains, which are the cytokine binding domain of this receptor protein, as a mask for IL-12 binding site (FIG. 1). The inventors propose an innovative platform for engineering masked IL-12 and IFNs (type I and II), which stay latent during circulation and residence in healthy organs but become activated once in the tumor. They do so by exploiting a high affinity interaction between a cytokine and a subunit of its receptor. When fused together via a tumor protease-sensitive linker, the cytokine-receptor fusion protein acts as a prodrug, remaining inactive in the periphery. Unmasking occurs at the tumor site, either in the tumor extracellular matrix (ECM) or on the tumor cell surface by tumor-associated proteases. This approach will greatly reduce the incidence of irAEs associated with cytokine immunotherapy and enhance the intratumoral inflammation, and sensitizing tumors to CPI therapy, resulting in both increased treatment tolerability and efficacy.

### B. Results

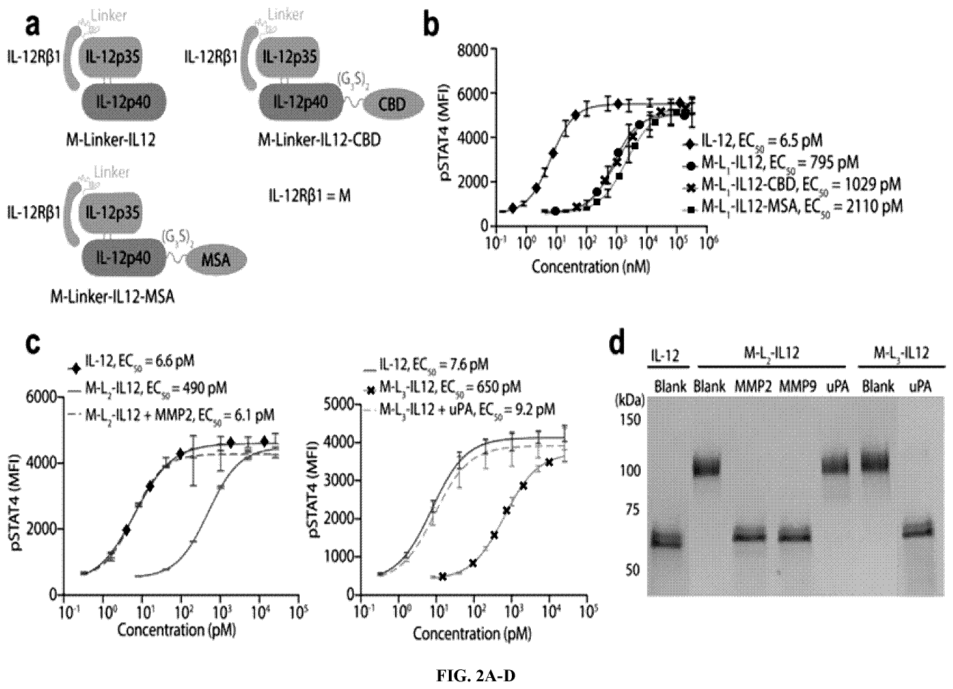

Design of ‘proIL12’ molecules via fusion to IL-12Rb1: The inventors have designed the IL-12Rb1 fibronectin I and II domains recombinant fusion to IL-12 protein. Proteins were expressed by HEK293 cells and then purified by Histidine tag-affinity purification and size-exclusion column purification. Latency can be conferred to cytokines upon fusion of domains that inhibit cytokine-receptor interaction (49,50). It is hypothesized that fusion of part of the murine IL-12Rb1 (Q20-A261, will be referred as the Mask, “M”) to IL-12 would render it inactive. This fragment is heavily glycosylated and has a molecular weight (MW) of 27 kDa. By exploiting the heterodimeric structure of IL-12, the inventors fused M to the N-terminus of mouse p35 subunit and co-transfected either with mouse p40, p40-CBD (to increase tumor retention) or p40-MSA (mouse serum albumin; to increase serum half-life) plasmids (FIG. 2A). The optimal linker length between the p35 subunit and IL-12Rb1 domain was determined by varying the number of repeats of (G3S)n and n=11 repeats yielded the purest and most stable fusion protein (M-L1-IL12, L₁ refers to (G3S)11 linker). Linkers shorter than that (n=2 and n=5) resulted in poorly folded proteins (data not shown). The inventors were also able to express M-L1-IL12-CBD and M-L1-IL12-MSA proteins. They then tested these constructs for their ability to phosphorylate STAT4 and compared the half-maximal effective concentration (EC50) to that of unmodified IL-12 (FIG. 2B). Masking of IL-12 provided a ˜100-fold decrease in activity, supporting the choice of IL12Rb1 as a masking agent. We then expressed several cleavable variants of masked IL-12 by replacing (G3S)11 linker with reported substrates of MMPs and serine proteases. M-L2-IL12 contains 3 repeats of MMP2,9-sensitive VPLSLYSG (SEQ ID NO:134) (VP) substrate and M-L3-IL12 contains 3 repeats of uPA-, matriptase- and legumain-sensitive LSGRSDNH (SEQ ID NO:49) (LS) substrate. Upon incubation of protease-sensitive masked IL-12 variants with respective proteases, activity was fully restored (FIG. 2C). This was further visualized via SDS-polyacrylamide gel electrophoresis (SDS-PAGE) (FIG. 2D). Molecular weight of proIL12 decreases from ˜100 kDa to MW of IL-12, indicating complete cleavage of the mask. These results suggest that proIL12 molecules have greatly decreased biological activity thanks to fusion with the portion of IL12Rb1 yet binding to the native IL12R complex is fully restored upon treatment of masked constructs with proteases, thus showing favorable masking and un-masking.

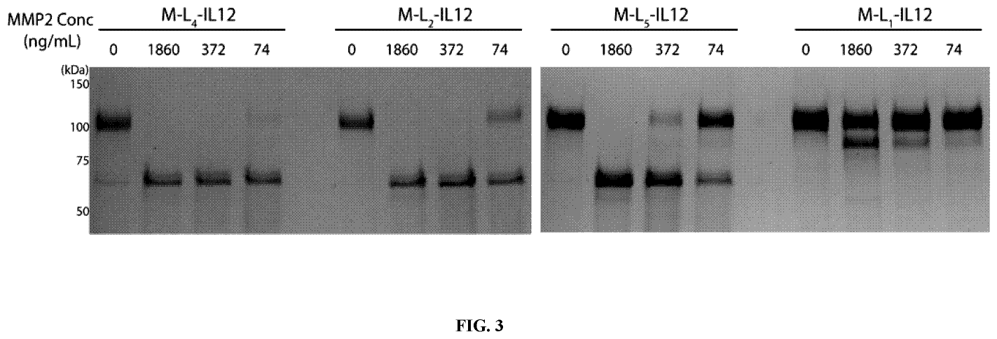

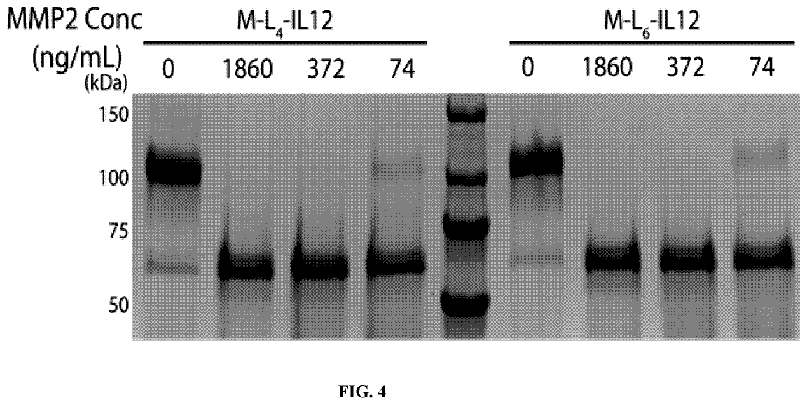

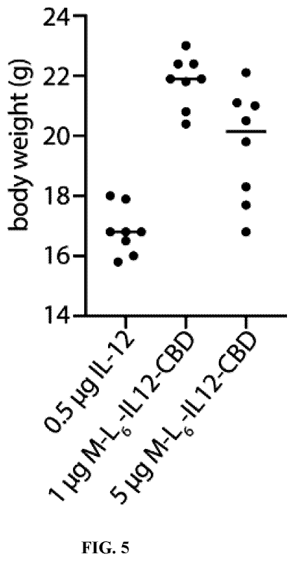

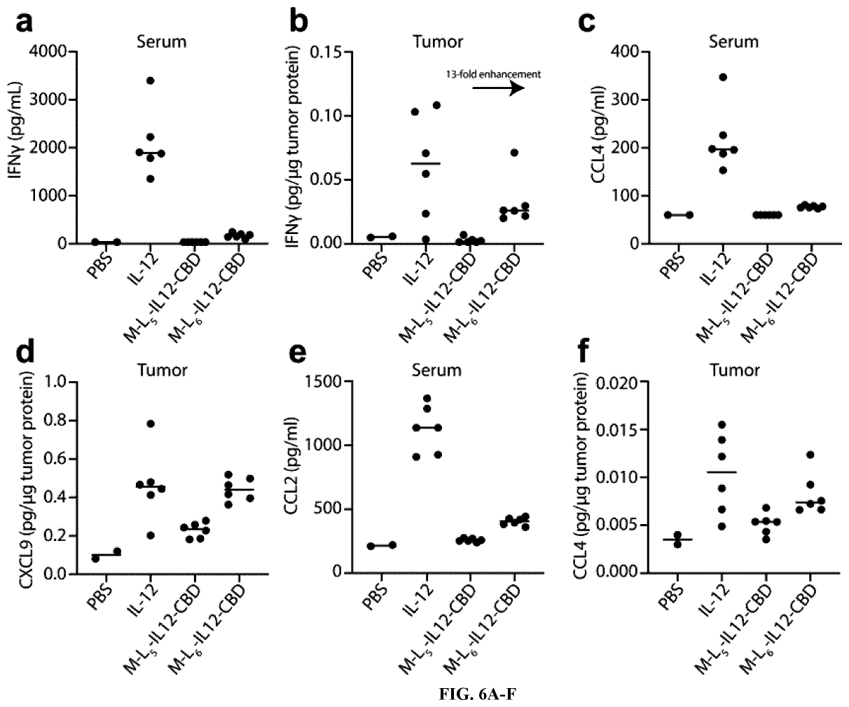

Linker Engineering: Given that many different linker designs are possible, the inventors explored whether it would be possible to increase linker efficiency in vitro against MMP2 (FIG. 3). Linker containing 3 repeats of HPVGLLAR (SEQ ID NO:138—“HP”), termed L4, was almost fully cleaved at the concentration of 74 ng/mL MMP2, whereas the L2 linker was only partially cleaved at that MMP2 concentration. Furthermore, the linker L5 (containing 1 repeat of VPLSLYSG—SEQ ID NO:134) was even less sensitive to MMP2 cleavage, indicating that increasing repeats of the substrate improves the cleavage kinetics. The inventors designed a linker with 2 repeats of each of HPVGLLAR (SEQ ID NO: 138) and VPLSLYSG (SEQ ID NO:134) and added one serine protease sensitive LSGRSDNH (SEQ ID NO:49)—termed L6, to further diversify the repertoire of proteases. The resulting L6 linker, was as sensitive to MMP2 as the L4 linker (FIG. 4). To determine whether the improvement in sensitivity translates into improved antitumor efficacy without exacerbating toxicity, we treated B16F10 melanoma-bearing mice with PBS, IL-12, M-L5-IL12-CBD (low sensitivity linker) or M-L6-IL12-CBD (high sensitivity linker), and quantified serum and intratumoral inflammatory cytokine levels (FIG. 6). ProIL12 molecules induced very little serum IFNg, a key mediator of toxicity (FIG. 5A). However, the intratumoral levels of IFNg were 13-fold higher in mice receiving high sensitivity linker than in the ones receiving low sensitivity linker (FIG. 5B). Serum levels of other proinflammatory chemokines such as CCL2 and CCL4 were greatly diminished by proIL12 treatment when compared to IL-12 (FIG. 5C,E). M-L6-IL12-CBD treatment induced similar level of CXCL9 as in IL-12-treated mice, a chemokine important for effector T cell recruitment (FIG. 5D). Cumulatively, these data demonstrate that proIL12 molecule bearing a high sensitivity linker is able to induce considerable intratumoral inflammation without causing toxicity. Furthermore, these studies point out that linker sensitivity is crucial for generating adequate intratumoral inflammation.

Toxicity Assessment of proIL12 in healthy mice: Studies in B16F10-melanoma bearing mice indicated that fusion of a masking agent is able to drastically decrease circulating proinflammatory biomarkers. The inventors sought to investigate the effect of daily proIL12 treatment in healthy C3H/HeJ mice, which are hyperresponsive to low doses of IL-12 treatment (15,53). These mice exhibit physical changes, such as weight loss and lethargy upon daily administration of IL-12. We treated C3H/HeJ mice daily for 8 days with 0.5 mg IL-12, 1 mg M-L6-IL12-CBD or 5 mg M-L6-IL12-CBD (on IL-12 basis) and measured their weight the day after the last dose (FIG. 6). These results illustrate that, even when dosed at 10-fold molar excess, proIL12 is still less toxic than unmodified IL-12. It is also contemplated that the linkers of this example will provide for more favourable toxicological readouts such as systemic liver transaminase levels, pancreas damage levels and complete blood counts.

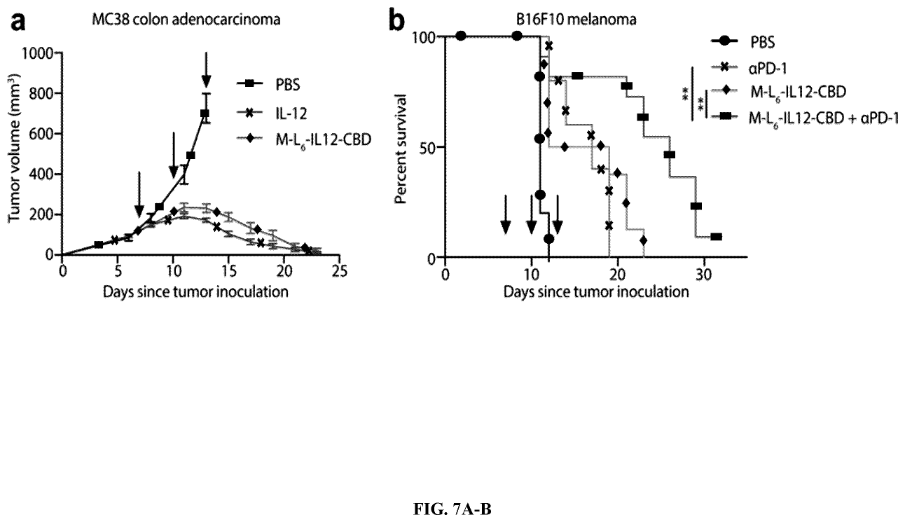

Antitumor efficacy of proIL12 in syngeneic mouse models of cancer: In order to demonstrate that administration of proIL12 can lead to tumor regression or tumor control, the inventors tested the engineered molecules in a rapidly growing, MMP-expressing MC38 colon adenocarcinoma model (FIG. 7A). Mice bearing established tumors were treated with PBS, 5 mg IL-12 or 15 mg M-L6-IL12-CBD (3-fold molar excess) 3 times every 3 days starting on day 7 post-tumor inoculation. Both IL-12 and M-L6-IL12-CBD resulted in complete survival of all treated mice, whereas all PBS-treated mice died by day 13. These results confirmed that proIL12 is efficacious as a single-agent and its efficacy is similar to IL-12 in an immune-infiltrated tumor model. We also evaluated the antitumor effects of proIL12 in combination with a checkpoint-blocking antibody, aPD-1, in B16F10 melanoma model (FIG. 7B). Although both proIL12 and aPD-1 demonstrated some tumor control, combination of the two drugs significantly prolonged the survival of the mice. Together, these data demonstrate that proIL12 induces antitumor effects and can synergize with a checkpoint blocking antibody in syngeneic tumor models.

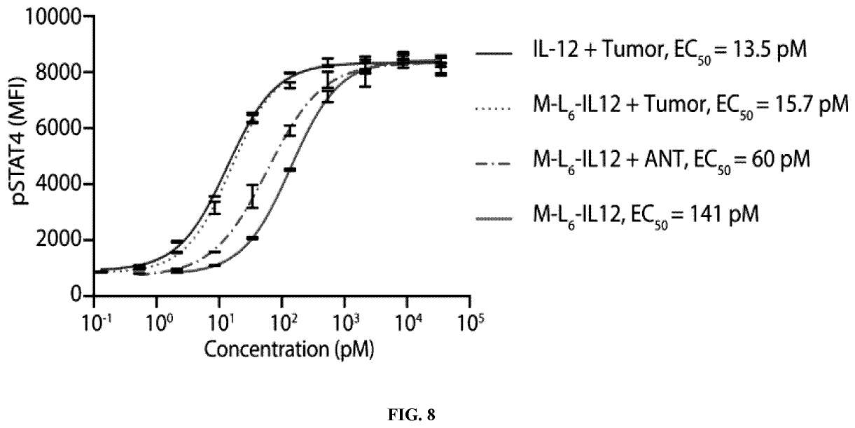

Cleavage of proIL12 by human tumor homogenates: The inventors sought to investigate whether proIL12 can be cleaved by a tumor lysate, and whether the cleaved product can bind to IL12R complex and activate cell signaling. To test this, they obtained flash frozen human breast cancer biopsies, as well as a biopsy of the ANT (nonmalignant tissue) from the same patient. The tumor and the ANT were homogenized in an assay buffer commonly used for MMP activation assays and total protein concentration of the lysates was normalized. The inventors incubated proIL12 either with the tumor lysate, ANT lysate or assay buffer alone. We also incubated unmodified IL-12 with the tumor lysate (FIG. 8). Incubation of proIL12 with the human tumor lysate, but not ANT lysate, resulted in activation of proIL12 and induced phosphorylation of STAT4 in the same level as unmodified IL-12 did. Although incubation of proIL12 with ANT resulted in minor activation as compared to proIL12 incubated with buffer only, this is perhaps due to nonspecific degradation of the linker/mask by the intracellular proteases. Nevertheless, these results indicate that human tumor homogenates are able to cleave proIL12 and that activated product can agonize the IL12R complex.

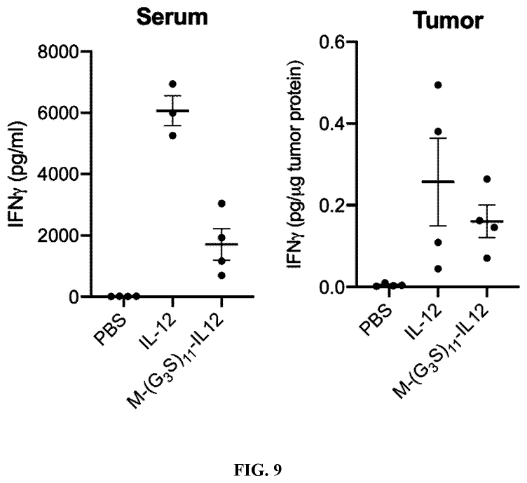

Engineered IFNg partial agonists decrease levels of STAT1 phosphorylation and bias towards the anti-cancer activity by upregulation of MHC-I without PD-L1: IFNγ is a highly pleiotropic cytokine that is a central coordinator of innate and adaptive immunity, through its actions on a wide range of immune cell types, including macrophages, DCs, and T cells (54). IFNγ is a homodimer (55), engaging two IFNγR1 receptors and two IFNγR2 receptors, which are constitutively expressed on all nucleated cells. Recent work from Prof. Mendoza describes the first complete structure of the hexameric IFNγ-receptor complex which provided a blueprint for breaking the pleiotropy of IFNγ (56). Structure-based designs of IFNγ variants alter the number and strength of binding to the receptors bound on a cell to tune down the strength of the cell signal. Reduction in the phospho-STAT1 signal by the partial agonists results in complete upregulation of the MHC-I necessary for antigen presentation on the tumor but limited expression of programmed death ligand 1 (PD-L1) which limits the activity of immune cells. Therefore, the partial agonists are biased towards the anti-cancer properties along the MHC-I:PD-L1 axis. These effects were demonstrated to work broadly on all cells tested which included seven cancer cell lines and three immune cell populations (FIG. 9). Because these findings were based on the human IFNγ protein, which does not cross-react with the mouse receptors, the inventors must first design a mouse version of the partial agonists. Design of the human analogs of the mouse partial agonists is a challenge because there are no structures of the mouse IFNγ ligand or receptors. Additionally, the sequence identity between the two species is low at 39%. Their first approach was to use a homology model from the human receptor complex where we predicted and screened 13 mouse variants. A mouse melanoma cell line, B16F10, was dosed with 2.5 or 62.5 nM of either wild-type or the mutants but no reduction in MHC-I or PD-L1 was detected by surface staining (data not shown). This experiment suggested engineering using a homology approach would be a challenge and new approaches should be employed.

### C. Conclusion

In summary, these studies demonstrate the value of masked cytokines, achieved through fusion with structurally-selected domains of cytokine-specific receptors. Masked cytokines can be administered via i.v. infusion and become activated preferentially in the tumor. The CBD prolongs retention in the tumor. We show substantially reduced incidence of irAEs following administration of masked cytokines, while anti-cancer effects are maintained. This sets the stage for further detailed analysis of masked proinflammatory cytokines, converging with the end goal of treating tumors that are otherwise refractory to conventional immunotherapies. In conclusion, we have developed a technology to reduce cytokine toxicity by fusing cytokine receptor to cytokine. Tumor specific protease responsive site fusion activates the cytokine within tumor. To further improve CBD-IL-12 therapy, fused a domain of the IL-12 receptor IL-12Rb1 to the IL-12, to form IL-12Rb1-IL-12. This fusion is inactive, but inclusion of a MMP or uPA cleavage site between the receptor mask and the cytokine yields a pro-cytokine that can be activated in the tumor microenvironment. We have demonstrated that the immunotoxicity of the IL-12 is thus reduced, and that the IL-12Rb1-IL-12 fusion with the protease-sensitive linker retains therapeutic utility.

### D. Materials and Methods

Production and purification of recombinant fusion protein of VWF A3 domain, IL-12Rb1 and IL-12: Protein production and purification were performed as described previously (57). The sequence encoding for the human VWF A3 domain residues Cys1670-Gly1874 (907-1111 of mature VWF), mouse/human IL-12, mouse/human IL-12Rb1 FNI-II domains, the fusion proteins were synthesized and subcloned into the mammalian expression vector pcDNA3.1(+) by Genscript. A sequence encoding for 6 His (SEQ ID NO:87) was added at the N-terminus for further purification of the recombinant protein. Suspension-adapted HEK-293F cells were routinely maintained in serum-free FreeStyle 293 Expression Medium (Gibco). On the day of transfection, cells were inoculated into fresh medium at a density of 1×106 cells/ml. 2 μg/ml plasmid DNA, 2 μg/ml linear 25 kDa polyethylenimine (Polysciences), and OptiPRO SFM media (4% final concentration, Thermo Fisher) were sequentially added. The culture flask was agitated by orbital shaking at 135 rpm at 37° C. in the presence of 5% CO2. 7 days after transfection, the cell culture medium was collected by centrifugation and filtered through a 0.22 μm filter. Culture media was loaded into a HisTrap HP 5 ml column (GE Healthcare), using an ÄKTA pure 25 (GE Healthcare). After washing of the column with wash buffer (20 mM imidazole, 20 mM NaH2PO4, 0.5 M NaCl, pH 7.4), protein was eluted with a gradient of 500 mM imidazole (in 20 mM NaH2PO4, 0.5 M NaCl, pH 7.4). The elusion solution was further purified with size exclusion chromatography using a HiLoad Superdex 200PG column (GE healthcare). All purification steps were carried out at 4° C. The expression of laminin LG domain was determined by western blotting using anti-His tag antibody (BioLegend) and the proteins were verified as >90% pure by SDS-PAGE.

Methods for in vitro cleavage: Recombinant mouse matrix metalloproteinase-2 (MMP2), MMP9 and recombinant human urokinase plasminogen activator (uPA) were purchased from R&D. Since MMP2 and MMP9 were supplied in their zymogen form, MMPs were first activated using 1 mM p-aminophenylmercuric acetate (APMA, Sigma) for 2 hr at 37° C. Following activation, MMPs and cytokines were diluted in an assay buffer containing 150 mM NaCl, 50 mM Tris, 10 mM CaCl2, 0.05% Brij-35 at pH=7.5. Final concentrations of MMP2, MMP9 and cytokine were 2 μg/mL, 5 μg/mL and 50 μg/mL, respectively. Cleavage conducted for 30 min at 37° C. Samples were then analyzed via gel electrophoresis. Cleavage using uPA was conducted according to manufacturer's protocol. Concentration of uPA was 10 g/mL.

Mice and cell lines: The mice and cell lines were prepared as described previously (58). C57BL/6 and C3H/HeJ age 8 to 12 weeks, were obtained from the Charles River laboratories. Experiments were performed with approval from the Institutional Animal Care and Use Committee of the University of Chicago. B16F10 cells were obtained from the American Type Culture Collection and cultured according to the instructions. All cell lines were checked for mycoplasma contamination by a pathogen test IMPACT I (IDEXX BioResearch).

Methods for pSTAT4 assessment: Mouse CD8+ T cells were purified from spleens of C57BL/6 mice using EasySep mouse CD8+ T cell isolation kit (Stem Cell). Purified CD8+ T cells (106 cells/mL) were activated in six-well plates precoated with 2 μg/mL α-CD3 (clone 17A2, Bioxcell) and supplemented with soluble 5 μg/mL α-CD28 (clone 37.51, BioLegend) and 30 ng/ml mouse IL-2 (Peprotech) for 3 days. Culture medium was IMDM (Gibco) containing 10% heat-inactivated FBS, 1% Penicillin/Streptomycin and 50 μM 2-mercaptoethanol (Sigma Aldrich). After 3 days of culture, activated CD8+ T cells were rested for 6 hrs in fresh culture medium and were transferred into 96-well plates (50,000 cells/well). Indicated amounts of IL-12 or proIL12 variants were applied to CD8+ T cells for 20 min at 37° C. to induce STAT4 phosphorylation. Cells were fixed immediately using BD Phosflow Lyse/Fix buffer for 10 min at 37° C. and then permeabilized with BD Phosflow Perm Buffer III for 30 min on ice. Cells were stained with Alexa Fluor (AF) 647-conjugated antibody against pSTAT4 (clone 38, BD) recognizing phosphorylation of Tyr693. Staining was performed for 1 hr at room temperature (RT) in the dark. Cells were acquired on BD LSR and data were analyzed using FlowJo (Treestar). Mean Fluorescence Intensity (MFI) of pSTAT4+ population was plotted against cytokine concentration. Dose-response curve was fitted using Prism (v8, GraphPad).

Plasma cytokine concentration analysis: The measurement is performed as described previously (58). 5×105 B16F10 melanoma cells were injected intradermally on left side of the back of each 8 week old C57BL/6 mouse. After 7 days, mice received indicated amounts of IL-12 and of proIL-12 variants. 2 days after IL-12 injection, blood samples were collected in heparinized tubes containing EDTA, followed centrifugation. Cytokine concentration in plasma was measured by LEGENDPLex Kit (BioLegend) according to the manufacture's protocol.

Anti-tumor efficacy of IL-12Rb1-IL-12 on B16F10 tumor: The measurement is performed as described previously (58). A total of 5×105 B16F10 cells re-suspended in 30 μL of PBS were inoculated intradermally on the left side of the back of each C57BL/6 mouse. After 7 days, mice were injected with IL-12 (5 μg), or IL-12Rb1-IL-12 (15 μg on IL-12 basis) i.v. Tumors were measured with a digital caliper starting 7 days after tumor inoculation, and volumes were calculated as ellipsoids, where V=4/3×3.14×depth/2×width/2×height/2. Mice were sacrificed at the point when either tumor volume had reached over 500 mm3.

### Example 2—Addition of Serum Proteins for Prolonged Circulation

Pro-cytokine can be improved by CBD-fusion to yield prolonged residence in tumors and/or albumin fusion to yield prolonged circulation. Cytokines generally have a very short half-life in the blood (9). Because pro-cytokine technology is relying on the protease within the body (i.e. tumor), it is important to increase the retention time of injected pro-cytokine within tumor. The inventors employ two approaches to improve the CBD-cytokine platform. The first step is to fuse collagen binding domain to the pro-cytokines. As described in Example 1, CBD can target and retain the fused protein within the tumor due to the nature of the tumor vasculature. Thus, the activity of CBD-pro-cytokine is more specific within the tumor, resulting in enhanced efficacy and safety. This is a form of a dual tumor targeting system.

Another step is to extend the pro-cytokine blood half-life. Because extended blood half-life of injected cytokines will allow more chance to contact tumor tissues, it is hypothesized that the efficacy of CBD-pro-cytokines would be further enhanced. This can be achieved by fusing albumin to CBD-pro-cytokine or pro-cytokine. Thus, these additional aspects of a tumor targeted cytokine with extended blood half-life, which is active only within the tumor microenvironment, are further contemplated.

### Example 3: Non-Cleavable Masked Cytokines

### A. Background

Attenuation of cytokine bioactivity can provide leverage in terms of toxicity, thereby increasing the tolerability and the therapeutic index. Cytokine masking be achieved via fusion of domains that inhibit cytokine-receptor interaction. Our strategy employs the fusion of a receptor subunit domain (termed the mask, ‘M’) to the cytokine of interest to decrease the bioactivity. The masking domain and the cytokine are connected via a flexible linker (in our case, (G₃S)ₙ where n can range from n=3 to n=15). In a previous invention, we taught the use of enzymatically-cleavable linkers; here, we make the surprising discovery that a non-cleavable, flexible linker can attenuate activity in a manner that leads to lower systemic toxicity yet still high intra-tumoral immune activation and anti-cancer effects.

### B. Results

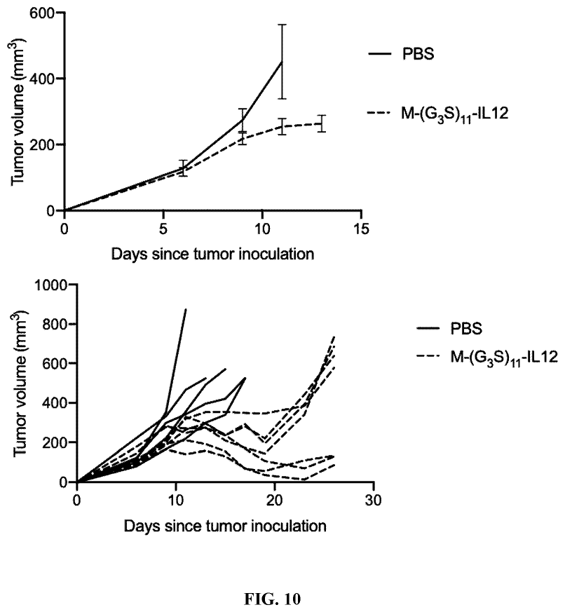

To evaluate the decrease of systemic toxicity of the non-cleavable, attenuated masked IL-12, we treated B16F10 melanoma-bearing mice either with saline (PBS), 5 μg IL-12 (wild-type) or 15 μg M-(G₃S)₁₁-IL12 (non-cleavable masked IL-12). 2 days following treatment with cytokines, we bled the mice to measure serum IFNγ as a biomarker of systemic toxicity (FIG. 9, left). On the third day post-treatment, we collected the tumors and measured intratumoral IFNγ as an indicator of antitumor efficacy (FIG. 9, right). Attenuated, non-cleavable M-(G₃S)₁₁-IL12 significantly reduced plasma IFNγ yet still maintained intratumoral inflammation as compared to unmodified IL-12. We then evaluated whether M-(GsS)₁₁-IL12 is capable of driving an antitumor response in the MC38 colon adenocarcinoma model (FIG. 10). Non-cleavable M-(G₃S)₁₁-IL12 exerted a stronger antitumor immune response when compared to saline treatment.

| | |
|---|---|
| Mouse M-(G₃S)₁₁-p35 | QLGASGPGDCCVEKTSFPEGASGSPLGPRNLSCYRVSKTDYECSWQ |
| | YDGPEDNVSHVLWCCFVPPNHTHTGQERCRYFSSGPDRTVQFWEQ |
| | DGIPVLSKVNFWVESRLGNRTMKSQKISQYLYNWTKTTPPLGHIKV |
| | SQSHRQLRMDWNVSEEAGAEVQFRRRMPTTNWTLGDCGPQVNSG |
| | SGVLGDIRGSMSESCLCPSENMAQEIQIRRRRRLSSGAPGGPWSDWS |
| | MPVCVPPEVLPQAGGGSGGGSGGGSGGGSGGGSGGGSGGGSGG |
| | GSGGGSGGGSGGGSRVIPVSGPARCLSQSRNLLKTTDDMVKTARE |
| | KLKHYSCTAEDIDHEDITRDQTSTLKTCLPLELHKNESCLATRETSS |
| | TTRGSCLPPQKTSLMMTLCLGSIYEDLKMYQTEFQAINAALQNHNH |
| | QQIILDKGMLVAIDELMQSLNHNGETLRQKPPVGEADPYRVKMKL |
| | CILLHAFSTRVVTINRVMGYLSSAHHHHHH (SEQ ID NO: 247) |
| Mouse p40 | MWELEKDVYVVEVDWTPDAPGETVNLTCDTPEEDDITWTSDQRH |
| | GVIGSGKTLTITVKEFLDAGQYTCHKGGETLSHSHLLLHKKENGIW |
| | STEILKNFKNKTFLKCEAPNYSGRFTCSWLVQRNMDLKFNIKSSSSS |
| | PDSRAVTCGMASLSAEKVTLDQRDYEKYSVSCQEDVTCPTAEETLP |
| | IELALEARQQNKYENYSTSFFIRDIIKPDPPKNLQMKPLKNSQVEVS |
| | WEYPDSWSTPHSYFSLKFFVRIQRKKEKMKETEEGCNQKGAFLVE |
| | KTSTEVQCKGGNVCVQAQDRYYNSSCSKWACVPCRVRS (SEQ ID |
| | NO: 6) |
| Human M-(G₃S)₁₁-p35 | CRTSECCFQDPPYPDADSGSASGPRDLRCYRISSDRYECSWQYEGPT |
| | AGVSHFLRCCLSSGRCCYFAAGSATRLQFSDQAGVSVLYTVTLWV |
| | ESWARNQTEKSPEVTLQLYNSVKYEPPLGDIKVSKLAGQLRMEWE |
| | TPDNQVGAEVQFRHRTPSSPWKLGDCGPQDDDTESCLCPLEMNVA |
| | QEFQLRRRQLGSQGSSWSKWSSPVCVPPEGGGSGGGSGGGSGGG |
| | SGGGSGGGSGGGSGGGSGGGSGGGSGGGSGRNLPVATPDPGMF |
| | PCLHHSQNLLRAVSNMLQKARQTLEFYPCTSEEIDHEDITKDKTSTV |
| | EACLPLELTKNESCLNSRETSFITNGSCLASRKTSFMMALCLSSIYED |
| | LKMYQVEFKTMNAKLLMDPKRQIFLDQNMLAVIDELMQALNFNSE |
| | TVPQKSSLEEPDFYKTKIKLCILLHAFRIRAVTIDRVMSYLNASHHH |
| | HHH (SEQ ID NO: 248) |
| Human p40 | IWELKKDVYVVELDWYPDAPGEMVVLTCDTPEEDGITWTLDQSSE |
| | VLGSGKTLTIQVKEFGDAGQYTCHKGGEVLSHSLLLLHKKEDGIWS |
| | TDILKDQKEPKNKTFLRCEAKNYSGRFTCWWLTTISTDLTFSVKSSR |
| | GSSDPQGVTCGAATLSAERVRGDNKEYEYSVECQEDSACPAAEESL |
| | PIEVMVDAVHKLKYENYTSSFFIRDIIKPDPPKNLQLKPLKNSRQVE |
| | VSWEYPDTWSTPHSYFSLTFCVQVQGKSKREKKDRVFTDKTSATVI |
| | CRKNASISVRAQDRYYSSSWSEWASVPCS (SEQ ID NO: 4) |

Amino acid sequences of mouse and human non-cleavable masked IL-12 variants. The functional IL-12 heterodimer is obtained via the co-transfection of the subunits encoding wild-type p35 (or masked p35) and p40.

Although certain embodiments have been described above with a certain degree of particularity, or with reference to one or more individual embodiments, those skilled in the art could make numerous alterations to the disclosed embodiments without departing from the scope of this invention. Further, where appropriate, aspects of any of the examples described above may be combined with aspects of any of the other examples described to form further examples having comparable or different properties and addressing the same or different problems. Similarly, it will be understood that the benefits and advantages described above may relate to one embodiment or may relate to several embodiments. Any reference to a patent publication or other publication is a herein a specific incorporation by reference of the disclosure of that publication. The claims are not to be interpreted as including means-plus- or step-plus-function limitations, unless such a limitation is explicitly recited in a given claim using the phrase(s) “means for” or “step for,” respectively.

### REFERENCES

The following references and the publications referred to throughout the specification, to the extent that they provide exemplary procedural or other details supplementary to those set forth herein, are specifically incorporated herein by reference.
1. Haslam, A. & Prasad, V. Estimation of the Percentage of US Patients With Cancer Who Are Eligible for and Respond to Checkpoint Inhibitor Immunotherapy Drugs. JAMA Network Open 2, e192535-e192535 (2019).
2. Hodges, T. R., et al. Mutational burden, immune checkpoint expression, and mismatch repair in glioma: implications for immune checkpoint immunotherapy. Neuro-Oncology 19, 1047-1057 (2017).
3. Rodig, S. J., et al. MHC proteins confer differential sensitivity to CTLA-4 and PD-1 blockade in untreated metastatic melanoma. Sci Transl Med 10(2018).
4. Gide, T. N., et al. Distinct Immune Cell Populations Define Response to Anti-PD-1 Monotherapy and Anti-PD-1/Anti-CTLA-4 Combined Therapy. Cancer Cell 35, 238-255.e236 (2019).
5. Ayers, M., et al. IFN-γ-related mRNA profile predicts clinical response to PD-1 blockade. J Clin Invest 127, 2930-2940 (2017).
6. Gao, J., et al. Loss of IFN-γ Pathway Genes in Tumor Cells as a Mechanism of Resistance to Anti-CTLA-4 Therapy. Cell 167, 397-404.e399 (2016).
7. Liu, D., et al. Integrative molecular and clinical modeling of clinical outcomes to PD1 blockade in patients with metastatic melanoma. Nat Med 25, 1916-1927 (2019).
8. Langrish, C. L., et al. IL-12 and IL-23: master regulators of innate and adaptive immunity. Immunol Rev 202, 96-105 (2004).
9. Szabo, S. J., Dighe, A. S., Gubler, U. & Murphy, K. M. Regulation of the interleukin (IL)-12R beta 2 subunit expression in developing T helper 1 (Th1) and Th2 cells. J Exp Med 185, 817-824 (1997).
10. Kobayashi, M., et al. Identification and purification of natural killer cell stimulatory factor (NKSF), a cytokine with multiple biologic effects on human lymphocytes. J Exp Med 170, 827-845 (1989).
11. Grohmann, U., et al. IL-12 acts directly on DC to promote nuclear localization of NF-kappaB and primes DC for IL-12 production. Immunity 9, 315-323 (1998).
12. Nagayama, H., et al. IL-12 responsiveness and expression of IL-12 receptor in human peripheral blood monocyte-derived dendritic cells. J Immunol 165, 59-66 (2000).
13. Miller, C. H., Maher, S. G. & Young, H. A. Clinical Use of Interferon-gamma. Ann N Y Acad Sci 1182, 69-79 (2009).
14. Garris, C. S., et al. Successful Anti-PD-1 Cancer Immunotherapy Requires T Cell-Dendritic Cell Crosstalk Involving the Cytokines IFN-γ and IL-12. Immunity 49, 1148-1161.e1147 (2018).
15. Leonard, J. P., et al. Effects of single-dose interleukin-12 exposure on interleukin-12-associated toxicity and interferon-gamma production. Blood 90, 2541-2548 (1997).
16. Kurzrock, R., Quesada, J. R., Rosenblum, M. G., Sherwin, S. A. & Gutterman, J. U. Phase I study of i.v. administered recombinant gamma interferon in cancer patients. Cancer Treat Rep 70, 1357-1364 (1986).
17. Atkins, M. B., et al. Phase I evaluation of intravenous recombinant human interleukin 12 in patients with advanced malignancies. Clin Cancer Res 3, 409-417 (1997).
18. Garnick, M. B., et al. Phase I/II study of recombinant interferon gamma in advanced renal cell carcinoma. J Urol 139, 251-255 (1988).
19. Mansurov, A., et al. Collagen-binding IL-12 enhances tumour inflammation and drives the complete remission of established immunologically cold mouse tumours. Nat Biomed Eng 4, 531-543 (2020).
20. Sin, W. X., Li, P., Yeong, J. P. & Chin, K. C. Activation and regulation of interferon-β in immune responses. Immunol Res 53, 25-40 (2012).
21. Borden, E. C. Interferons α and β in cancer: therapeutic opportunities from new insights. Nat Rev Drug Discov 18, 219-234 (2019).
22. Platanias, L. C. Mechanisms of type-I- and type-II-interferon-mediated signalling. Nat Rev Immunol 5, 375-386 (2005).
23. Darnell, J. E., Jr., Kerr, I. M. & Stark, G. R. Jak-STAT pathways and transcriptional activation in response to IFNs and other extracellular signaling proteins. Science 264, 1415-1421 (1994).
24. Meinke, A., Barahmand-Pour, F., Wöhrl, S., Stoiber, D. & Decker, T. Activation of different Stat5 isoforms contributes to cell-type-restricted signaling in response to interferons. Mol Cell Biol 16, 6937-6944 (1996).
25. Stark, G. R., Kerr, I. M., Williams, B. R., Silverman, R. H. & Schreiber, R. D. How cells respond to interferons. Annu Rev Biochem 67, 227-264 (1998).
26. Mattei, F., Schiavoni, G., Belardelli, F. & Tough, D. F. IL-15 Is Expressed by Dendritic Cells in Response to Type I IFN, Double-Stranded RNA, or Lipopolysaccharide and Promotes Dendritic Cell Activation. The Journal of Immunology 167, 1179-1187 (2001).
27. Golomb, H. M., et al. Alpha-2 interferon therapy of hairy-cell leukemia: a multicenter study of 64 patients. J Clin Oncol 4, 900-905 (1986).
28. Groopman, J. E., et al. Recombinant alpha-2 interferon therapy for Kaposi's sarcoma associated with the acquired immunodeficiency syndrome. Ann Intern Med 100, 671-676 (1984).
29. Kantarjian, H. M., O'Brien, S., Anderlini, P. & Talpaz, M. Treatment of myelogenous leukemia: current status and investigational options. Blood 87, 3069-3081 (1996).
30. Wang, Y. S., et al. Identification of the major positional isomer of pegylated interferon alpha-2b. Biochemistry 39, 10634-10640 (2000).
31. Bukowski, R. M., et al. Treating cancer with PEG Intron: pharmacokinetic profile and dosing guidelines for an improved interferon-alpha-2b formulation. Cancer 95, 389-396 (2002).
32. Hollingsworth, R. E. & Jansen, K. Turning the corner on therapeutic cancer vaccines, npj Vaccines 4, 7 (2019).
33. Ishihara, J., et al. Targeted antibody and cytokine cancer immunotherapies through collagen affinity. Sci Transl Med 11(2019).
34. Fischer, A. Mechanism of the Proteolytic Activity of Malignant Tissue Cells. Nature 157, 442-442 (1946).
35. Kessenbrock, K., Plaks, V. & Werb, Z. Matrix metalloproteinases: regulators of the tumor microenvironment. Cell 141, 52-67 (2010).
36. Ulisse, S., Baldini, E., Sorrenti, S. & D'Armiento, M. The urokinase plasminogen activator system: a target for anti-cancer therapy. Curr Cancer Drug Targets 9, 32-71 (2009).
37. Uhland, K. Matriptase and its putative role in cancer. Cell Mol Life Sci 63, 2968-2978 (2006).
38. Ardi, V. C., Kupriyanova, T. A., Deryugina, E. I. & Quigley, J. P. Human neutrophils uniquely release TIMP-free MMP-9 to provide a potent catalytic stimulator of angiogenesis. Proc Natl Acad Sci USA 104, 20262-20267 (2007).
39. Nakamura, E. S., Koizumi, K., Kobayashi, M. & Saiki, I. Inhibition of lymphangiogenesis-related properties of murine lymphatic endothelial cells and lymph node metastasis of lung cancer by the matrix metalloproteinase inhibitor MMI270. Cancer Sci 95, 25-31 (2004).
40. Liu, S. C., et al. Relationships between the level of matrix metalloproteinase-2 and tumor size of breast cancer. Clin Chim Acta 371, 92-96 (2006).
41. Schmalfeldt, B., et al. Increased expression of matrix metalloproteinases (MMP)-2, MMP-9, and the urokinase-type plasminogen activator is associated with progression from benign to advanced ovarian cancer. Clin Cancer Res 7, 2396-2404 (2001).
42. Werle, B., et al. Cathepsin B, plasminogenactivator-inhibitor (PAI-1) and plasminogenactivator-receptor (uPAR) are prognostic factors for patients with non-small cell lung cancer. Anticancer Res 24, 4147-4161 (2004).
43. Remacle, A., Murphy, G. & Roghi, C. Membrane type I-matrix metalloproteinase (MT1-MMP) is internalised by two different pathways and is recycled to the cell surface. Journal of Cell Science 116, 3905-3916 (2003).
44. Cui, G., Cai, F., Ding, Z. & Gao, L. MMP14 predicts a poor prognosis in patients with colorectal cancer. Hum Pathol 83, 36-42 (2019).
45. Yao, Q., Kou, L., Tu, Y. & Zhu, L. MMP-Responsive ‘Smart’ Drug Delivery and Tumor Targeting. Trends Pharmacol Sci 39, 766-781 (2018).
46. Dai, Z., Yao, Q. & Zhu, L. MMP2-Sensitive PEG-Lipid Copolymers: A New Type of Tumor-Targeted P-Glycoprotein Inhibitor. ACS Appl Mater Interfaces 8, 12661-12673 (2016).
47. Desnoyers, L. R., et al. Tumor-Specific Activation of an EGFR-Targeting Probody Enhances Therapeutic Index. Science Translational Medicine 5, 207ra144-207ra144 (2013).
48. Trang, V. H., et al. A coiled-coil masking domain for selective activation of therapeutic antibodies. Nature Biotechnology 37, 761-765 (2019).
49. Adams, G., Vessillier, S., Dreja, H. & Chernajovsky, Y. Targeting cytokines to inflammation sites. Nat Biotechnol 21, 1314-1320 (2003).
50. Gerspach, J., et al. Restoration of membrane TNF-like activity by cell surface targeting and matrix metalloproteinase-mediated processing of a TNF prodrug. Cell Death Differ 13, 273-284 (2006).
51. Turk, B. E., Huang, L. L., Piro, E. T. & Cantley, L. C. Determination of protease cleavage site motifs using mixture-based oriented peptide libraries. Nat Biotechnol 19, 661-667 (2001).
52. Spranger, S., Dai, D., Horton, B. & Gajewski, T. F. Tumor-Residing Batf3 Dendritic Cells Are Required for Effector T Cell Trafficking and Adoptive T Cell Therapy. Cancer Cell 31, 711-723.e714 (2017).
53. Huber, V. C., et al. Delivery of IL-12 intranasally leads to reduced IL-12-mediated toxicity. Int Immunopharmacol 3, 801-809 (2003).
54. Ni, L. & Lu, J. Interferon gamma in cancer immunotherapy. Cancer Med 7, 4509-4516 (2018).
55. Yphantis, D. A. & Arakawa, T. Sedimentation equilibrium measurements of recombinant DNA derived human interferon gamma. Biochemistry 26, 5422-5427 (1987).
56. Mendoza, J. L., et al. Structure of the IFN gamma receptor complex guides design of biased agonists. Nature 567, 56-+(2019).
57. Martino, M. M., et al. Growth factors engineered for super-affinity to the extracellular matrix enhance tissue healing. Science 343, 885-888 (2014).
58. Ishihara, J., et al. Matrix-binding checkpoint immunotherapies enhance antitumor efficacy and reduce adverse events. Sci Transl Med 9(2017).

1. A polypeptide comprising a cytokine linked to a masking agent through a linker, wherein the linker comprises a glycine-serine linker; and wherein the masking agent comprises a cytokine receptor polypeptide or fragment thereof that specifically binds to the cytokine.

2. The polypeptide of claim 1, wherein the linker comprises SEQ ID NO:48.

3. The polypeptide of claim 2, wherein the polypeptide comprises SEQ ID NO:248.

4. A polypeptide comprising a cytokine linked to a masking agent through a linker, wherein the linker comprises at least 2 protease cleavage sites of SEQ ID NO: 138 and at least two protease cleavage sites of SEQ ID NO: 134, and wherein the masking agent comprises a cytokine receptor polypeptide or fragment thereof that specifically binds to the cytokine.

5. A polypeptide comprising a cytokine linked to a masking agent through a linker, wherein the linker comprises an amino acid sequence with at least 80% sequence identity to one of SEQ ID NOS:48, 103-108, or 219-246, and wherein the masking agent comprises a cytokine receptor polypeptide or fragment thereof that specifically binds to the cytokine.

6. The polypeptide of claim 5, wherein the linker comprises an amino acid sequence of one of SEQ ID NOS:48, 103-108, or 219-246.

7. The polypeptide of any one of claims 1-6, wherein the cytokine comprises IL12 and wherein the masking agent comprises IL12R polypeptide or an IL12-binding fragment thereof.

8. The polypeptide of claim 7, wherein the IL12 comprises one or both of the p35 and p40 subunits.

9. The polypeptide of claim 8, wherein the IL12 comprises the p35 and p40 subunits linked through a disulfide bond.

10. The polypeptide of claim 8, wherein the IL12 comprises the p35 and p40 subunits linked through a peptide linker.

11. The polypeptide of any one of claims 7-10, wherein the IL12 comprises a polypeptide of SEQ ID NO:3 or a polypeptide with at least 80% sequence identity to SEQ ID NO:3.

12. The polypeptide of any one of claims 7-11, wherein the IL12R polypeptide or fragment comprises IL12Rβ1, or a fragment thereof.

13. The polypeptide of claim 12, wherein the IL12Rβ1 polypeptide comprises human FNI-II domain of IL-12Rβ1.

14. The polypeptide of claim 13, wherein the IL12Rβ1 polypeptide comprises a polypeptide of SEQ ID NO: 195 or a polypeptide having at least 80% sequence identity to SEQ ID NO:195.

15. The polypeptide of claim 14, wherein the polypeptide has at least 80% sequence identity to SEQ ID NO: 195 and wherein the polypeptide binds to IL12.

16. The polypeptide of any one of claims 4-15, wherein the polypeptide comprises SEQ ID NO:197 or a polypeptide with at least 80% sequence identity to SEQ ID NO:197.

17. The polypeptide of any one of claims 7-15, wherein the masking agent is fused to the N-terminus of the p35 subunit of IL12, and wherein the linker is between the masking agent and the p35 subunit of IL12.

18. The polypeptide of any one of claims 7-13, wherein the masking agent is fused to the C-terminus of the p40 subunit of IL12, and wherein the linker is between the masking agent and the p40 subunit of IL12.

19. The polypeptide of any one of claims 1-6, wherein the cytokine comprises IL-2 and the masking agent comprises IL-2Rα, IL-2Rβ, IL-2Rγ, fragments, or combinations of fragments thereof.

20. The polypeptide of claim 19, wherein the cytokine comprises the amino acid sequence of SEQ ID NO:23 or an amino acid sequence with at least 80% sequence identity to SEQ ID NO:23.

21. The polypeptide of claim 19 or 20, wherein the masking agent comprises the amino acid sequence of SEQ ID NO:27, 29, 31 or a fragment or combination of SEQ ID NO:27, 29, or 31.

22. The polypeptide of any one of claims 19-21, wherein the masking agent comprises an amino acid sequence with at least 80% sequence identity to SEQ ID NO:27, 29, and/or 31.

23. The polypeptide of any one of claims 1-6, wherein the cytokine comprises IFNγ and the masking agent comprises IFNγR1, IFNγR2, fragments, or combinations of fragments thereof.

24. The polypeptide of claim 23, wherein the cytokine comprises the amino acid sequence of SEQ ID NO:26 or an amino acid sequence with at least 80% sequence identity to SEQ ID NO:26.

25. The polypeptide of claim 23 or 24, wherein the masking agent comprises the amino acid sequence of SEQ ID NO:33, 35 or a fragment or combination of SEQ ID NO:33 and/or 35.

26. The polypeptide of any one of claims 23-25, wherein the masking agent comprises an amino acid sequence with at least 80% sequence identity to SEQ ID NO:33 and/or 35.

27. The polypeptide of any one of claims 4-26, wherein the linker further comprises at least one serine protease sensitive cleavage site.

28. The polypeptide of claim 27, wherein the serine protease sensitive cleavage site comprises a cleavage site of SEQ ID NO:47 or SEQ ID NO:3.

29. The polypeptide of claim 28, wherein the linker comprises SEQ ID NO:219 or a polypeptide with at least 80% sequence identity to SEQ ID NO:219.

30. The polypeptide of any one of claims 1-29, wherein the linker comprises or further comprises (GGGS)n, wherein n is 1-4.

31. The polypeptide of claim 29, wherein n is 2.

32. The polypeptide of any one of claims 4-31, wherein the linker comprises SEQ ID NO:220 or a polypeptide with at least 80% sequence identity to SEQ ID NO:220.

33. The polypeptide of any one of claims 1-32, wherein the cytokine comprises a pro-inflammatory cytokine.

34. The polypeptide of any one of claims 1-33, wherein the polypeptide is conjugated to a tumor targeting agent.

35. The polypeptide of claim 35, wherein the tumor targeting agent comprises an antibody or an antigen-binding fragment thereof.

36. The polypeptide of claim 35, wherein the antibody or antigen-binding fragment comprises a stroma targeting antibody or stroma-binding fragment thereof.

37. The polypeptide of claim 36, wherein the antibody or binding fragment specifically binds to fibronectin, alternatively spliced domains of fibronectin, collagens, tenascins, periostins, syndecans, proteoglycans, or a tumor stroma cell-specific antigen.

38. The polypeptide of claim 37, wherein the antibody or binding fragment specifically binds toe extra domain A (EDA) or extra domain B (EDB) of fibronectin.

39. The polypeptide of claim 37, wherein the tumor targeting agent comprises a Fab that specifically binds to an alternatively spliced domain of fibronectin comprising extra domain A (EDA).

40. The polypeptide of claim 35, wherein the tumor targeting agent comprises an antibody or antigen binding fragment thereof that specifically binds to a tumor-associated antigen.

41. The polypeptide of claim 34, wherein the tumor targeting agent comprises a collagen binding domain.

42. The polypeptide of claim 41, wherein the polypeptide comprises at least two collagen binding domains.

43. The polypeptide of claim 41 or 42, wherein the polypeptide comprises a collagen binding domain from decorin or von Willebrand factor (VWF).

44. The polypeptide of claim 43, wherein the collagen binding domain comprises a polypeptide comprising SEQ ID NO:1 or an amino acid sequence with at least 80% sequence identity to SEQ ID NO:1.

45. The polypeptide of any one of claims 1-43, wherein the polypeptide further comprises a serum protein conjugated to the polypeptide.

46. The polypeptide of claim 45, wherein the serum protein is conjugated to the polypeptide through a peptide bond.

47. The polypeptide of claim 45 or 46, wherein the serum protein comprises albumin.

48. The polypeptide of any one of claims 4-47, wherein the polypeptide comprises a second linker.

49. The polypeptide of claim 48, wherein the second linker comprises glycine and serine amino acid residues.

50. The polypeptide of claim 49, wherein the linker comprises SEQ ID NO:47 or SEQ ID NO:191.

51. A polypeptide comprising the amino acid sequence of one of SEQ ID NOS:248 or 197-218, or 247 or an amino acid sequence with at least 80% sequence identity to one of SEQ ID NOS:248 or 197-218, or 247.

52. The polypeptide of any one of claims 4-51, wherein the polypeptide comprises a protein tag.

53. The polypeptide of any one of claims 4-52, wherein the polypeptide is not operatively linked to a particle, nanovesicle, or liposome.

54. A pharmaceutical composition comprising the polypeptide of any one of claims 4-53.

55. The pharmaceutical composition of claim 54, wherein the composition does not comprise a liposome, particle, or nanovesicle.

56. The pharmaceutical composition of claim 55, wherein the composition further comprises an additional polypeptide.

57. The pharmaceutical composition of claim 56, wherein the additional polypeptide comprises an IL-12 p35 or p40 subunit.

58. The pharmaceutical composition of claim 57, wherein the additional polypeptide comprises a polypeptide of SEQ ID NO:3 or 4 or an amino acid sequence with at least 70% sequence identity to SEQ ID NO:3 or 4.

59. A nucleic acid encoding for the polypeptide of any one of claims 4-53.

60. A host cell comprising the nucleic acid of claim 59.

61. A method for making a polypeptide comprising expressing the nucleic acid of claim 59 in a cell and isolate the expressed polypeptide.

62. A method for treating cancer comprising administering the polypeptide of any one of claims 4-53 or the composition of any one of claims 54-58.

63. The method of claim 62, wherein the cancer comprises melanoma, colon, or breast cancer.

64. The method of claim 63, wherein the cancer comprises melanoma.

65. The method of any one of claims 62-64, wherein the cancer comprises a solid tumor.

66. The method of any one of claims 62-65, wherein the method further comprises administration of one or more additional cancer therapies.

67. The method of any one of claims 62-66, wherein the subject has or will receive an immunotherapy.

68. The method of any one of claims 62-67, wherein the method further comprises administration of an immunotherapy.

69. The method of claim 67 or 68, wherein the immunotherapy comprises an immune checkpoint inhibitor.

70. The method of claim 69, wherein the immune checkpoint inhibitor comprises an anti-PD-1 monoclonal antibody or an anti-CTLA-4 monoclonal antibody.

71. The method of claim 70, wherein the immune checkpoint inhibitor comprises one or more of nivolumab, pembrolizumab, pidilizumab, ipilimumab or tremelimumab.

72. The method of any one of claims 68-71, wherein the immunotherapy is administered before, after, or concurrent with the polypeptide.

73. The method of any one of claims 62-72, wherein the polypeptide or composition is administered systemically.

74. The method of any one of claims 62-72, wherein the polypeptide or composition is administered intratumorally.

75. The method of claim 73 or 74, wherein the polypeptide or composition is administered by intravenous injection.

76. The method of any one of claims 62-75, wherein the subject has been previously treated with a cancer therapy.

77. The method of claim 76, wherein the subject has been determined to be non-responsive to the previous treatment or wherein the wherein the subject experienced non-specific toxicity to the previous treatment.

78. The method of any one of claims 62-77, wherein the method further comprises administration of an additional polypeptide.

79. The method of claim 78, wherein the additional polypeptide comprises a IL-12 p35 or p40 subunit.

80. The method of claim 79, wherein the additional polypeptide comprises a polypeptide of SEQ ID NO:3 or 4 or an amino acid sequence with at least 70% sequence identity to SEQ ID NO:3 or 4.
